SEQUENCE LISTING (25 sequences)

SEQ ID NO: 1 - DNA, 2325 bp, Mus musculus
  CDS (1)...(1146)
atgggaccccagaacagaaacactagctttgcaccagacttgaatccaccccaagaccatgtctccttaaactacagttatggtgattatgacctccccctgggtgaggatgaggatgtgaccaagacacagaccttctttgcagccaaaattgtcattggcgtggcactggcaggcatcatgctggtctgcggcattggcaactttgtcttcattgctgccctcgcccgctacaagaagctgcgcaaccttaccaacctcctcattgctaacctggccatctctgacttcctggtggcgatcgtctgctgcccctttgagatggactattatgtagtacggcagctttcctgggcgcatggtcacgtgctttgtgcctccgtcaactaccttcgtacggtctccctgtacgtctccaccaacgctctgctggccatcgctattgacagatacctcgctattgtccaccctttgaaaccacggatgaattatcagaccgcttccttcctgatcgctttggtctggatggtctccatcctcatcgctgtcccatctgcctacttcaccacagaaaccatcctcgttatcgtcaagaatcaagaaaaaatcttctgtggtcagatctggtcggtggaccagcagctctactacaaatcctacttcctcttcgtcttcgggcttgagttcgtgggtcccgtggtcactatgaccctgtgctatgccaggatctcccaagagctctggttcaaggctgtacctggcttccagacggagcaaatccgcaagcggctgcgttgccgccgcaagacagtgctactgctcatgggcatcctcacagcctacgtgctgtgctgggcgccgttctatggctttaccatagtgcgagacttcttccccacggtagttgtgaaggagaagcactacctcaccgccttctacgtcgtggagtgcattgccatgagcaacagcatgatcaatactatatgcttcgtgacggtcaagaacaacaccatgaaatacttcaagaagatgctgcggctccactggcggccctctcactacgggagtaagtccagcgctgacctcgacctcaaaaccagcggggtgcctgccactgaagaggtggattgtatcagactaaagtagccttcaggtgttgcccaaggaaaaatttaacattcggtactcagtaaatcacacaccatcaaccactcacaagctacatggaaagatacggctgtattcacgttctcctgctctaatgtatcaggacgcttctatgtaataacatacagcacaactgatgtctgcataacatcttagaaggcagacacaaatagtaacaagtgatgtggactgaatgcttctgtctgcaaaccacaccaaccaattattcaaggacaagagctgacatgtgagaattacctgctatgtgcaaaaacaagttacccccccaaaaaatgatagaagctatttggagttattcagctctatctatctatctatctatccatccatccatccatccatccaggtcactagaaagaagtcacaaatgactagccagagtcatgctacatattctttcattctgtatcttttctgcacagaactgtcaaaggcaatagaataaagcacctagacatactagaaatgtaaggataactccatcaatagggagaccaaggcctcataggaagagggtccatatagtatactgactttccccactccacaccagttatctccttagatattctgtacttatctgcaatgttgtaatttcaaatgaggaaaaataaggggacaggctttaccacagatgtatcaaatctcatcaagcccatagggcaaagatgggaggctcctgacacaagaaatgtatccagttctggataactttaatgccaagcatttcagggctctggggtcttggaggaagaggacacagaaagagccgaggtttccagtggcaatgagtataatctgtccatttgctatgatttggacaattttctagaacatactccgacttacaaaaggaactctacttgagatccaaagatccgggtaaaagtcctaaccccaggactcatctctgtgtgtctccactgtaatgaaatggaaataatgaaaacggatcattaggaacatcagcccggcgaagtcatggtgtggatgtgattttcacctcttcctttgtgaagaatgaggtcgtgaaaagctcattagagggagtttggaatggagaaacagctccacacttttcatccctcttctttgaatcggagaccactaaacgcatctttgaagtagcgtatctat

SEQ ID NO: 2 - protein, 381 aa, Mus musculus
MGPQNRNTSFAPDLNPPQDHVSLNYSYGDYDLPLGEDEDVTKTQTFFAAKIVIGVALAGIMLVCGIGNFVFIAALARYKKLRNLTNLLIANLAISDFLVAIVCCPFEMDYYVVRQLSWAHGHVLCASVNYLRTVSLYVSTNALLAIAIDRYLAIVHPLKPRMNYQTASFLIALVWMVSILIAVPSAYFTTETILVIVKNQEKIFCGQIWSVDQQLYYKSYFLFVFGLEFVGPVVTMTLCYARISQELWFKAVPGFQTEQIRKRLRCRRKTVLLLMGILTAYVLCWAPFYGFTIVRDFFPTVVVKEKHYLTAFYVVECIAMSNSMINTICFVTVKNNTMKYFKKMLRLHWRPSHYGSKSSADLDLKTSGVPATEEVDCIRLK

SEQ ID NO: 3 - DNA, 2556 bp, Mus musculus
  CDS (1)...(1182)
  misc_feature 1308, 1439, 1440: n = A,T,C or G
atggagaccactgtcggggctctgggtgagaataccacagacaccttcaccgacttcttttctgcactcgatggccatgaagcccaaaccggctcgttaccattcactttcagctacggtgactatgacatgcccctggatgaagaggaagatgtgaccaattctcggactttctttgctgccaagattgtcattggcatggctttggtgggtatcatgctagtgtgtggcatcggcaacttcatctttatcactgccctggcccgctacaaaaagctccgcaacctcaccaacctgcttatcgccaacctggccatttcagacttcctcgtggccatcgtgtgctgcccctttgagatggactactatgtggtgcgccagctctcctgggagcatggtcatgtcctgtgcgcctctgtcaactacttgcgtaccgtctccctctacgtctccactaacgccctactggccattgccattgacaggtatctggccattgtgcacccgctgagaccgcggatgaagtgtcaaacagccgccggcctgatcttcctggtgtggtcagtatccatcctcatcgccattccagctgcctacttcaccactgagaccgtgctggtcatcgtggagagacaggagaagatcttctgtggtcagatctggccggtggatcagcagttctactacaggtcctatttccttttggttttcggcctcgagttcgtgggccccgtagtcgccatgaccttgtgctatgccagggtgtcccgggagctctggttcaaggcggtgccaggcttccagacagagcagatccgccggacggtgcgctgccgccgcaggacggtgctggggctcgtgtgcgtcctctctgcctatgtgctgtgctgggctcccttctatggcttcactatcgtgcgtgacttcttcccctccgtgtttgtgaaggagaagcactacctcaccgccttctatgtggtggagtgcatcgccatgagcaacagcatgatcaatacgctctgctttgtgactgtcaggaataacaccagtaagtacctcaagaggatcctgcggcttcagtggagggcctctcccagcgggagcaaggccagcgctgacctcgacctcaggaccacgggaatacctgccaccgaggaggtggactgcatccgactgaaataagcaaaatggtaccacagcgcccgggtcgcacacagcagccatgaacttgtttttctgcggaaggcagaggaaagagacaactacttagacacgctattcaaggaccactgaagtgtggaatttctnaaatggagacctgagatactgtcactagtggtcagggttcaccgaatatctaatttttgcaaagactagacaccagtgaaagtcctttgacaaagtaaagtggggattatagcaaggaaatatggattgtnntgtctggaaaaggctatttccagagagaaggcagagcaggctatgtaggactctcctgtctgtgcttagggtggagtacagggctctgtgtgggcctagctcacctctggcaggactcttgggtttttgtttatgtctagcattagcaagcccggagcaagagtatggaagaggttcccagatgtgctgtagtccttttctgcttacctcagaatccccactgaggggaatcagaagcatgagtggacactccaggacccacggctaagttctgcgcatccacccccaacctacggtcagatcttaggcctagggctctgcacacagacagtgggggaggggacttctttggagcaccgacttgagggaggagccacttggactctgggtgccatttgcatagggaatttggggttctgagtcataagtttgagttcaggtgagcatgattcccccactcatggtctactcactgcaggggaaaaggaggtgaagaaggggagggtcaggtgggacccctgaaggcatgcgtgcaggttaaaggatgctgttgcttcatctctcagcatctgtgccctggaccactcgtgcttcttgagacttcatgcctctgagtttatggagcatagactcatgaaggtaaacgtagcaccttgctgtcccgttcccactagctcccaaacaccggtgttttcatctcaggcagcctatttgatgggattccctaactggtaagactgggagaaagatttcttaaccagtggtacctgactcttgacctggtcatggtcctgggaatgaagacttgctgctctttccaaatgaaggtgaccttggagaaatcaccacatttctgctcttcagcgcacatgaagacttgcacatcaggggtgctgcctggacactcaggaaaccgaagatgaccaacagtgagctctagggaccccacagacttatgaaagtctcatcccatgtgataactcaccttcttagcttggcatttagaattctggcctgagaaaacctgaggcctagctttgactaagtcatctacacagacctgagataagactttctaaagataaaggtaatacttaagaaacctgttagaattgt

SEQ ID NO: 4 - protein, 393 aa, Mus musculus
METTVGALGENTTDTFTDFFSALDGHEAQTGSLPFTFSYGDYDMPLDEEEDVTNSRTFFAAKIVIGMALVGIMLVCGIGNFIFITALARYKKLRNLTNLLIANLAISDFLVAIVCCPFEMDYYVVRQLSWEHGHVLCASVNYLRTVSLYVSTNALLAIAIDRYLAIVHPLRPRMKCQTAAGLIFLVWSVSILIAIPAAYFTTETVLVIVERQEKIFCGQIWPVDQQFYYRSYFLLVFGLEFVGPVVAMTLCYARVSRELWFKAVPGFQTEQIRRTVRCRRRTVLGLVCVLSAYVLCWAPFYGFTIVRDFFPSVFVKEKHYLTAFYVVECIAMSNSMINTLCFVTVRNNTSKYLKRILRLQWRASPSGSKASADLDLRTTGIPATEEVDCIRLK

SEQ ID NO: 5 - protein, 81 aa, Homo sapiens
AVITGACDKDSQCGGGMCCAVSIWVKSIRICTPMGKLGDSCHPLTRKVPFFGRRMHHTCPCLPGLACLRTSFNRFICLAQK

SEQ ID NO: 6 - protein, 100 aa, Homo sapiens
AVITGACDKDSQCGGGMCCAVSIWVKSIRICTPMLGDSCHPLTRKNNFGNGRQERRKRKRSKRKKEVPFFGRRMHHTCPCLPGLACLRTSFNRFICLAQK

SEQ ID NO: 7 - protein, 81 aa, Mus musculus
AVITGACDKDSQCGGGMCCAVSIWVKSIRICTPMGQVGDSCHPLTRKVPFWGRRMHHTCPCLPGLACLRTSFNRFICLARK

SEQ ID NO: 8 - protein, 102 aa, Mus musculus
AVITGACDKDSQCGGGMCCAVSIWVKSIRICTPMGQVGDSCHPLTRKSHVANGRQERRRAKRRKRKKEVPFWGRRMHHTCPCLPGLACLRTSFNRFICLARK

SEQ ID NO: 9 - protein, 86 aa, Homo sapiens
AVITGACERDVQCGAGTCCAISLWLRGLRMCTPLGREGEECHPGSHKVPFFRKRKHHTCPCLPNLLCSRFPDGRYRCSMDLKNINF

SEQ ID NO: 10 - protein, 86 aa, Mus musculus
AVITGACERDIQCGAGTCCAISLWLRGLRLCTPLGREGEECHPGSHKIPFLRKRQHHTCPCSPSLLCSRFPDGRYRCFRDLKNANF

SEQ ID NO: 11 - protein, 77 aa, Xenopus laevis
AVITGACDKDVQCGSGTCCAASAWSRNIRFCIPLGNSGEDCHPASHKVPYDGKRLSSLCPCKSGLTCSKSGEKFKCS

SEQ ID NO: 12 - protein, 75 aa, Bufonidae sp.
AVITGACDKDVQCGSGTCCAASAWSRNIRFCIPLSGEDCHPASHKVPYDGKRLSSLCPCKSGLTCSKSGEKFKCS

SEQ ID NO: 13 - protein, 80 aa, Serpentes sp.
AVITGACERDLQCGKGTCCAVSLWIKSVRVCTPVGTSGEDCHPASHKIPFSGQRMHHTCPCAPNLACVQTSPKKFKCLSK

SEQ ID NO: 14 - DNA, 4038 bp, Homo sapiens
tcccatgagatggtaagctcccagggggctgggttcatgtctgttttgctcaccctcgtacccccaatgctgcacacaatatctggaatatagtaagtcttagttaatacctgttgaatgaataaattatggttttctacccacctaaaagaaaatattagcttacacatttactcactttcagagtctttgtataattctggtataaagaatgctgtactagaaaaaatacaaaggaaattacacattattatttataatagcaattactaatataattcaaagttcattatgctcattaaggagtccaagccatgaatgattcatttcctgtagaatatagcactgctcacctgataccttgttgtcttcagaaaccagtgacagacttgcactgtctgtccccttgaagacagattgtcatgtggcttgttttggtcactaaaaagtgagcaaaagtgacaggcatgaattccaggcagagaaagctggtgtacaaccctctggctgcacccaactatgttagtttcctgtgtctgccataatgaaatcaccacaactgggtggcttaaaacaatagaaacttattctttcgtggttctggaggctaaaagtccaaaaccaaggggctgacaaggccgtgctccctgtgaaggatctggggaagaatccttccttgctctttcagatcctgatggctgctggcaatccttggtgttccttgccttgtagaagcattgctccaatctctgcccccatcttcacttgatgttctcccgtgtgtgtggggggggtggggtggggggagtctagcctgtgtctctgtatccagctctccctcccatttctcttatgaagacaccagccattgaatttagaacccaccctaatatagcatgacctcatcttaagttgattacgtctgcaaaaaccctatttccaaataaggtcacgtccacaggtaccagggcttaggccttgaaaataacttgctaggggtcacagtataggccactacactggcaatcatgaagcacatgttgaaatggagccttatcagtcagcctgagtcccaaattgactgccgtgagacaggcctgtgtctcccagctcacttcatttcagtcacgtggcccccaagtaaagagacaggctgaatcatctgcaccaggtaaagcttccatctctcttgctttttggtgactacctttaacggattcatatacatgacatgagtctgtactgtatatgatcttctcgtaactgtctctggactgtccttcatgggacatattcctgttgctggtcacttactgaatgcctgccatatgcctattatcgtgctgtgtgccaagagggatgcatagaaaattagacacaaggatcctaatcatgaaaagtgtacaatctaatatagtgggcaggacagaaaagacttatatcagcattggctgaaattgtcaggggagaccaaataataaggcttaataagaggtaaccctaacagagctcccagtctgttccaggcactgtgataagcattttgcaggtattattaaattccataggggttgtaccccatgaggttggtattttataaccattttacaagaccggaaacagaggcttcaaaaggttgtgtaacttgcccagtggtcacacaggattccaatcctgatcagcctgtctcacaaacattgggttctatagacgctcctagattgcattttcgtttaagctgagccttgatggtctgctggaatatggtaggctacactttacacacacaaggctcatttcacctaatacagttatgcctgggcagaagtgatcatgtggcaatatcaacaggttacagtaatagaaaagaatcaataaactactgtttcatttctatgtcattgttgctaagttgtcccaactaccttttttaatggactaatccaaactctttttttttttcatttttccctttataacaattgaagtcagacttcatttttcaaacttggcctcagatacaaagatgacatatcaagagccttcatttctctcctaaagcattaaagcaattaaaatttccaaaagaacatgaaactaaacaaccctatttttaagtgtttccaaacttatttcttttttttaaacttgtttcaaaacagccttatgaggactgttttccaaacagctgtgtaagaagccagccacttttgaaatctgatttttcctgtgtagacatatcatattttctatgcttgaagaagcagggaatacccaagctggcattcaatagtagcgaatatgaaatagaccattaaaagaaagtcataggaatgttaaaatccatgttgactggtttttacatttacccggcagcattcccgagctagcgttggcatggagactggaaaaggaaactttccacaagtctgtcacttgctactgtttctacttactccactgtgagtccaattttaacatttttttaagttgaaaaaagggtttgactccttttgtgttttctgttcaaggcgttttttaaacatgagaacacgtgtgaaaaaggtttttaaaaatcagccaaagattggggtttccaaatattcagctgtttaacattcagataattgcctgccttccccccgctatcccccacatttcgtctgaatgctttggcacgggggagccccacagtctgaaagtaactctgtcccaactctccttactgcatttattaaagaggctgaaagacgcatctgcttttcaatagtttgcggttcttaagagaccaggaaagccagaggttctgactattcaggaagaaagttgggtttcccaaaacgggcaagcaagttcggtgtggtcacatctaggtattcttgacgtcatttttgttgagggaaacaggctgcagtttttggagccaggctaggatgagagatggagggcaaatctgtcacttattctttcctggtgtgcctggaacccactagacactcaattcatgtttacgtgaatgaaggaatcacaataacgccctatcgcatctgtaaaaccgaagggtgattttcctgggctggaaagtttaagaaagaagagagagctgcgtacctagggcttaaggggcctcaggctggcactcgaaaccaggcgttctcatttccctttggcatcaccctgaacggctgtgcctggagctggcgcggggctgaagaggggaggaaatacatgtgaggaaaatcagaggagagggtcgggaacagatgtgggcataaagggaaggcctctgacttgaaataaacaaataggagtcccgcagctggaaggacaaatcctttcacgtcgcgggtgatgtgggattggggcgattttgctctccctttgttctttcccctgccttccacgtttccagggtatttgattgatgtctgtcttctctgttaagttattccaccgtgaggagagagccggtggcagtacctggcacgcagcgggcgcccagtatagacctgctgaacaaacgaatggattcaggggctgctgtgtccccctcacccaccccccgccccctatgtgtccacagcgccccgcacgtagtaggcgacccctaaacatctgtacagcaaatgatttgcaagttttcggcgctgagcacgtggagctttggaaaccaggacagcaaatgagtgtctcggagaccacaaaagcggttccggcgcgtgcgaaaggcggtggctgggcgacggcggagggaacggcgcagagcggggcgccccgccgggagcgctgcctgcgtggcgcccgaggcgggggcgcggggggccgcgcatagcacgtgctcgtctgggagccggccgggccgaggcgggcgcgcgtgtgcgcgtggggcgtggggtgtgtgcccgcgccgtgccccccgcgtgtgctgccgggcgggcgccggcgtgagtcacggcggggctagcctttataacggcccggaggctcgcgggagccgccgcgcccgtccgcccgccgctccgcgctccacccagcgca

SEQ ID NO: 15 - DNA, 2800 bp, Homo sapiens
tgtactgtatatgatcttctcgtaactgtctctggactgtccttcatgggacatattcctgttgctggtcacttactgaatgcctgccatatgcctattatcgtgctgtgtgccaagagggatgcatagaaaattagacacaaggatcctaatcatgaaaagtgtacaatctaatatagtgggcaggacagaaaagacttatatcagcattggctgaaattgtcaggggagaccaaataataaggcttaataagaggtaaccctaacagagctcccagtctgttccaggcactgtgataagcattttgcaggtattattaaattccataggggttgtaccccatgaggttggtattttataaccattttacaagaccggaaacagaggcttcaaaaggttgtgtaacttgcccagtggtcacacaggattccaatcctgatcagcctgtctcacaaacattgggttctatagacgctcctagattgcattttcgtttaagctgagccttgatggtctgctggaatatggtaggctacactttacacacacaaggctcatttcacctaatacagttatgcctgggcagaagtgatcatgtggcaatatcaacaggttacagtaatagaaaagaatcaataaactactgtttcatttctatgtcattgttgctaagttgtcccaactaccttttttaatggactaatccaaactctttttttttttcatttttccctttataacaattgaagtcagacttcatttttcaaacttggcctcagatacaaagatgacatatcaagagccttcatttctctcctaaagcattaaagcaattaaaatttccaaaagaacatgaaactaaacaaccctatttttaagtgtttccaaacttatttcttttttttaaacttgtttcaaaacagccttatgaggactgttttccaaacagctgtgtaagaagccagccacttttgaaatctgatttttcctgtgtagacatatcatattttctatgcttgaagaagcagggaatacccaagctggcattcaatagtagcgaatatgaaatagaccattaaaagaaagtcataggaatgttaaaatccatgttgactggtttttacatttacccggcagcattcccgagctagcgttggcatggagactggaaaaggaaactttccacaagtctgtcacttgctactgtttctacttactccactgtgagtccaattttaacatttttttaagttgaaaaaagggtttgactccttttgtgttttctgttcaaggcgttttttaaacatgagaacacgtgtgaaaaaggtttttaaaaatcagccaaagattggggtttccaaatattcagctgtttaacattcagataattgcctgccttccccccgctatcccccacatttcgtctgaatgctttggcacgggggagccccacagtctgaaagtaactctgtcccaactctccttactgcatttattaaagaggctgaaagacgcatctgcttttcaatagtttgcggttcttaagagaccaggaaagccagaggttctgactattcaggaagaaagttgggtttcccaaaacgggcaagcaagttcggtgtggtcacatctaggtattcttgacgtcatttttgttgagggaaacaggctgcagtttttggagccaggctaggatgagagatggagggcaaatctgtcacttattctttcctggtgtgcctggaacccactagacactcaattcatgtttacgtgaatgaaggaatcacaataacgccctatcgcatctgtaaaaccgaagggtgattttcctgggctggaaagtttaagaaagaagagagagctgcgtacctagggcttaaggggcctcaggctggcactcgaaaccaggcgttctcatttccctttggcatcaccctgaacggctgtgcctggagctggcgcggggctgaagaggggaggaaatacatgtgaggaaaatcagaggagagggtcgggaacagatgtgggcataaagggaaggcctctgacttgaaataaacaaataggagtcccgcagctggaaggacaaatcctttcacgtcgcgggtgatgtgggattggggcgattttgctctccctttgttctttcccctgccttccacgtttccagggtatttgattgatgtctgtcttctctgttaagttattccaccgtgaggagagagccggtggcagtacctggcacgcagcgggcgcccagtatagacctgctgaacaaacgaatggattcaggggctgctgtgtccccctcacccaccccccgccccctatgtgtccacagcgccccgcacgtagtaggcgacccctaaacatctgtacagcaaatgatttgcaagttttcggcgctgagcacgtggagctttggaaaccaggacagcaaatgagtgtctcggagaccacaaaagcggttccggcgcgtgcgaaaggcggtggctgggcgacggcggagggaacggcgcagagcggggcgccccgccgggagcgctgcctgcgtggcgcccgaggcgggggcgcggggggccgcgcatagcacgtgctcgtctgggagccggccgggccgaggcgggcgcgcgtgtgcgcgtggggcgtggggtgtgtgcccgcgccgtgccccccgcgtgtgctgccgggcgggcgccggcgtgagtcacggcggggctagcctttataacggcccggaggctcgcgggagccgccgcgcccgtccgcccgccgctccgcgctccacccagcgca

SEQ ID NO: 16 - DNA, 7349 bp, Mus musculus
acacaagaaagaagagggaagagtgttctagaccatcctgaaagtagatagccatagatcagtggcccaggctctgcactactgttctctggcaagtcacatgtggaggctcagaaacatctgagatttgtggaaacaagtttaaagattgagtatatggaatactcccaccaaattgctgtgtcttacttctagttattggccacccaaggtttttatggcaatgttataaatttgaagagaaactctaacagttaaaagcttggcaccagagcacactgggtttaaatctagtttctttccttagcaattaccctggcttgagatggcatggttgacctttctaaattccactttgctcttccataatggccaagagttctacccagagggtccctggggtttttatagaagatgtactggaattcacaaagtacttactgtgtgcaacgcatagctctgcacttttgaaatgaaaacaccacaattggcttgtgccccaaaacaaagaaacaaaacaaaataaagctaggagtcataatgtgaaccctagttccctagcaagcctcctgcctcagcttcctgaatgctgggatttcaagctaagagccaccacatctggttataaaagttttgtacctattatttcatatgtatatgaatatgtcttgttacttcttctaggtcctttccttctttaaatgttgctgaatgaggtttatttcgctgtgatagatagttcctgttgacctgaaaggatgataaagtaactacctagtcatggtttttaatcttaagaaaacccctttcttaggtgagacttcttttttttccatctggaaagttaccaaatacgccacacatttgcataacaatctctttgaatgaagacagcagcgcacgcagcaccagcttttgcctttagggccatcttcaatagaaaaaaaataaaaaaaataaaaaagctgacagagaagagcagcgaacgttataatgagaacatctgaatcttttagaaccaaatgttcttcggtgacccttcatattatctcaaagtcacggtgtgctaattgcttgaagaaaaccctttctccgggattaatacttcagctatttaaaaatagataaactttactgtacattatcacttgaatggaacaaatgcttttcagtgcctccgtagacaattggcgttcacattgaatcctggtgatttattatttttttttaaataaagagaaaatcatcagcattgaactagattaaaatagacatcttatttagaattataaaagccattcctttttcatgtcacatttaaaatacggttattattttagaatgatagcacagatgatattataggtttaacaagaagagccacgtggaacagtgcctcaataaaggatgaaggcaaagtagaaaggagaatatccatcttatttcatttgactttttttttggaacaaatctaaatgaaaagtcatttacacaaaggcaaataaaacccagtaatgaaattaccgggcccacctattgtgttaccataataagccataagaaaaagacgcatgcaaagtaaggtttcagcaaagccagagtcactcttaacaacagagccatgtgaggaagcgtctggaacagtttcctcttctgtggctgagaaaatgcctgacactttccgtgcaagatggagagcaaggcctgtgtttcttgatctttctttgataacctcatcactcttgatgaagcatctactgtgtgctggatcattggaatcagttatagtaaggttaatatgcaaaatagcaatcagtaagccagaactttgggcaatatccgaaagtccaaaagaatgtggattacatcagtgagttggagtctgcgcagtacatctcacaggaagttcaagcaaagacagtggaggcaagatcagtaagagtttgtaaaactgcagtatgagacagtacagagactctaggctctttttttttttttaaacacatgaatctagaaacaacagagctggaaaatagcccagtcataagcacaacaaactaatttgctcactaggacccaatgtgaagcaagccagactgatgggtggtatacacacatcatccactgtgtttgttcacaagaacccgtgcaaaataagctggaccagcgatggtatagtatacacatatcatctcaggcagagacaggcagatctctggggctcgctggccagccaacctagcttacccggtgagtcccaggcctagtgaaagaccctatctcagaaaagaaggtagaaggctcttgaagaatgtcagctggggctgtcccctggcctccacatgcaaagacgcagtgtgtgcccacgaatgcgcacacacacacacacacaacgaggaatcagcaagctttctctacactagcagatagaagatattttttggcttttccaatcatgtggtctcacaagtactcagtaaaaagcagacacgggcaatacagaaacaggcatggctagatagaacataaaaaataaataatagatagatagatagatagatagatagatagatagatagatagatagatagaatactggctagttttgtgtcaacttgacacagctggagttatcacagagaggggagcttcagttggggaaatgcctccatgagatacaactgtaaggcattttctcaattagtgatcaagagggaaaggccccttgtgggtgggaccatctctgggctggtagtcttgggttctataagagagcaggctgagcaagccaggggaaggaagacagtaagtaacatccctccatggcctctgcattagcttctgcttcctgacctgcttgagttccagtcctgacttcctttggtgatgaagagcagtatggaagtgtaagccgaataaaccctttcctccccaacttgcttcttggtcatcatgtttgtacaagaatagaaaccctgactaagacagatagatagacaacaaaaagcaatgtgtcaacgttttcccagtttctactaaatcctagtcattgtggctacattctatgaaatgaaaaaattttaaagtccaacaagaagccactggttggccatttctttctttagctcagctgctccccctcgccctgcctgcctttgtccaatcccatgtaagcaccagagtccctcctgcgagccagttaccacagtgaggttcctccttcctccccactcccaatagttcctgcccttctttctcttgactgcccacaaggattctgcagtgctctgccttcaggagatggatggtgacttctctcagtcctccatcatgctcttctccacattcctagtgatttggtcagccaaaatcgagtgctttgttcaattcttggtacatagcttgtgcttagtaagtacctgctgaatgagtgaatgaatgatggtctattagttaaaacaaagcactctcagcttgcacatttgtactctttcaccatgtatcacttgaaggactaaacaaacaaatacagacacacattatcctttggagtaagaattactaaggttggccaaagtacagtggctcctttcctgtagcactgcttaccagatttctggtcttagcagcaactacatttgcactgctgtctttagcaaggtgaactgtcacgtgacccgtttccgtcactgtggcacagtcaggcttgagttccaggcagaggcagcagaaagctgaattgcaaccctccagccccacccttccttttagtttcccagaggttctaggagaagtgagcagaactaggtggcatcgcacatcagaggttattttcttgtagctctggatgttagaagtccaaaatcaaggatgctttccccagctcttctgaattctgaaagccagctgtgggggaggaggtgtgtcatatcagtctctatatccaaagatcttgcatctgttataaaaagatgggaaaatagcttagtcagcaaagggcttgcctcgaaaacattgatgtcctgagttcagtctccagaactcaaaaaagccaggtgcggtgacttgagtctgtggctacctgagtctgtaatcccaacaatggtaaaatgggaggccgaggcaggcagatttcctgaggctcacaggacagtgaggctgacctacatgtcaaagttccagtctagtgagagaccctgtcccaaacaaaaggtagaagccgtctggtactcaaggttgtctgacctgcacatatgctgtgtgtagaggtgcccacacatacacaaatgcatactcatgcatgcacagacacacacacacacaaaaccacactttttaaaaaaaatgacaaagatatcagttgttgggtttagggcatactctcatccagtatgacttcaagtttggttgatcatatcagcaaggaccctacttccaaataaagacacaggaaccaggagctagagtttcaacataactttctaggtgagacttagtataaccctagtctggaatcatcataaatcatcagttcaaataaagtccccctcagctgagctcccagagtgcctgctgtagcttgaatgtgtgacagctgtaagacatgtctccaggggcccctcattccagtcccatattcttggaggaaacagcaggcgaatcccctgcaccaggtctctctttccctctaagcttttgctgcctgcattcgtgatccatgtagattaagtaagtgcatctacactgtgtgattcttgcttctaacctctctctgtccctgatcacaaaatccactcctggtgctcatcaatcattgtctgagtgctggctaatgccacatgcaagaaggattttcaggagaaatggactcctagccagcacgtgtatgcgatctaattaggtgggcaggacagaaaacagccgtatcagtgttcaatgaacctatcagggaaaagcacatgataagatttaactagagtgcacccccttgcaggacttccaggctatcacagacttggtgcttagcactttcaaaggtgttctctttgagcttcatggtggctggaccaggaagttggtattttatgcccacagggagccagagaccttaagaggtcaaaggttcagttgccacaaaagactccaatccaggtcactctaactttcaaatgtagggttcttcgtatcacactgtcaaaccagctgagaggtggtagtcatgcaagactgccagctatatatactctgaacccacgaagttttggtcctcacccaaaggttgttcctggctaatttgaccacaatgactgatcgaaccaacaggttgcagtaacaaaacagaacttgaatttcattattgttttatcgttgtcttacggtggggaggagtgtaattcaaacagtgtctttccatttctaataaatgaaatcagacttgatctaacttacctgtggggtccaaagaagttctgttgaagagttttcatttcttctatcaagtattaaagccattaaacccttccaaagaggtttagggggtagccgagcagtagaattgacccaggatgtccatgcccttggttgcaacctcagcacaggaaaacagaagagccttaaaccagtcttatgtttaagcatctctaaccttgattcaaaagtcttaggagactgttttgggaacagtcaatctcaatggaaggttagcatttccctgggtagagatggcctgttttctacgctctgagaagtagggaatacccagttggcatttgatagtaacaaatatgaaaccgaccatttaagaaaagaattaaaagagtgtcaggaatgttaaagttcatgttgactggtttttacatttcccccgggctgcactcccatgccttcgttggcatggagactggaaaggaaactttccaccagtctgtcacttgctactgtttccacttagtttactgtgaatccacttttaacagttttctcaaagttaaaaaggagtttgatcccgggtgtgttttcggtctcaggatgcttcttgaactcaaaaacatggtacaaaaaggctttaaaactcagccaaagatctagggtttgaaacgcccagctgtttaacattcaaataattccctggccctttgttcattttttttcccctctacatttcgtctgaatgctttggcatgagggagcccacagactgaaagtaactgtggcccagctctccttactgtattgattaaagtggctgaaaaggcccatctgcctttcagcagtctgtggtcctatgggacatgggaaagtctaaggtccctagctgtctaggagtgaggtgggctcccccaaacctcagaaaaagtcaaattgatcatccttgtagaagcaagcaagatgagattattggagccaaattaggaacacagagagggttgtctcttgtctcatgggatgaatgcctagagtccagcagacacacctaagtgattgatcagaacccccaccaccaccaccaaaaaaaaatatatatatatgtataagacttggagtttcctaggctggagaaccgagctgggagagagccaccttcccagggcctcaggctggcagagcaagctgggagttctccttttttccccctttggcaacatcctgttctgcctgaagcaggctggaagctgcagaggggagggaacgcacgtgagagaaatcagggcagaaagggtcaggaacagatgtgggcaccggagaagtcatttccaaaaaggaaaggagactcccacagctggaggggcagccgagtccctccaacttcttaagagatgtgggacggggtgtgctggcgctttgttcttccagcctgctatgttcgcttgccctttgcttgtttgtctgttctttctatgttgtgccacctcagggcagagccggtggcagtacctggcacgcacggatctctcactgtagatatgctgaacaaatgtgtacaaatacagtgcagttgtgtccgccgcgtctggcacgtcgtgggtaccccctgcacatctgtatggcaaatgatgtgcctctgcgagtgtgggggctgagcacgtgaggctctggaaaacaggacggcgaaggaggagggtttctgagaccacaaaagcttcaggaaggctggctagggctgcggcgccccgcgggggctctgcccgcgtggcgctttgcgcgtggggcgcggggcacgtgcgcgtgtgcgcgtggagcgcggggtgtgtgcccgcgccgtgccccccgcgtgctgcctggcgtgagtcaccgcggggctcgcctttataaccgccgccaggctcgcagctccgcagagcagcccggcc

SEQ ID NO: 17 - DNA, 2800 bp, Mus musculus
cagctgagctcccagagtgcctgctgtagcttgaatgtgtgacagctgtaagacatgtctccaggggcccctcattccagtcccatattcttggaggaaacagcaggcgaatcccctgcaccaggtctctctttccctctaagcttttgctgcctgcattcgtgatccatgtagattaagtaagtgcatctacactgtgtgattcttgcttctaacctctctctgtccctgatcacaaaatccactcctggtgctcatcaatcattgtctgagtgctggctaatgccacatgcaagaaggattttcaggagaaatggactcctagccagcacgtgtatgcgatctaattaggtgggcaggacagaaaacagccgtatcagtgttcaatgaacctatcagggaaaagcacatgataagatttaactagagtgcacccccttgcaggacttccaggctatcacagacttggtgcttagcactttcaaaggtgttctctttgagcttcatggtggctggaccaggaagttggtattttatgcccacagggagccagagaccttaagaggtcaaaggttcagttgccacaaaagactccaatccaggtcactctaactttcaaatgtagggttcttcgtatcacactgtcaaaccagctgagaggtggtagtcatgcaagactgccagctatatatactctgaacccacgaagttttggtcctcacccaaaggttgttcctggctaatttgaccacaatgactgatcgaaccaacaggttgcagtaacaaaacagaacttgaatttcattattgttttatcgttgtcttacggtggggaggagtgtaattcaaacagtgtctttccatttctaataaatgaaatcagacttgatctaacttacctgtggggtccaaagaagttctgttgaagagttttcatttcttctatcaagtattaaagccattaaacccttccaaagaggtttagggggtagccgagcagtagaattgacccaggatgtccatgcccttggttgcaacctcagcacaggaaaacagaagagccttaaaccagtcttatgtttaagcatctctaaccttgattcaaaagtcttaggagactgttttgggaacagtcaatctcaatggaaggttagcatttccctgggtagagatggcctgttttctacgctctgagaagtagggaatacccagttggcatttgatagtaacaaatatgaaaccgaccatttaagaaaagaattaaaagagtgtcaggaatgttaaagttcatgttgactggtttttacatttcccccgggctgcactcccatgccttcgttggcatggagactggaaaggaaactttccaccagtctgtcacttgctactgtttccacttagtttactgtgaatccacttttaacagttttctcaaagttaaaaaggagtttgatcccgggtgtgttttcggtctcaggatgcttcttgaactcaaaaacatggtacaaaaaggctttaaaactcagccaaagatctagggtttgaaacgcccagctgtttaacattcaaataattccctggccctttgttcattttttttcccctctacatttcgtctgaatgctttggcatgagggagcccacagactgaaagtaactgtggcccagctctccttactgtattgattaaagtggctgaaaaggcccatctgcctttcagcagtctgtggtcctatgggacatgggaaagtctaaggtccctagctgtctaggagtgaggtgggctcccccaaacctcagaaaaagtcaaattgatcatccttgtagaagcaagcaagatgagattattggagccaaattaggaacacagagagggttgtctcttgtctcatgggatgaatgcctagagtccagcagacacacctaagtgattgatcagaacccccaccaccaccaccaaaaaaaaatatatatatatgtataagacttggagtttcctaggctggagaaccgagctgggagagagccaccttcccagggcctcaggctggcagagcaagctgggagttctccttttttccccctttggcaacatcctgttctgcctgaagcaggctggaagctgcagaggggagggaacgcacgtgagagaaatcagggcagaaagggtcaggaacagatgtgggcaccggagaagtcatttccaaaaaggaaaggagactcccacagctggaggggcagccgagtccctccaacttcttaagagatgtgggacggggtgtgctggcgctttgttcttccagcctgctatgttcgcttgccctttgcttgtttgtctgttctttctatgttgtgccacctcagggcagagccggtggcagtacctggcacgcacggatctctcactgtagatatgctgaacaaatgtgtacaaatacagtgcagttgtgtccgccgcgtctggcacgtcgtgggtaccccctgcacatctgtatggcaaatgatgtgcctctgcgagtgtgggggctgagcacgtgaggctctggaaaacaggacggcgaaggaggagggtttctgagaccacaaaagcttcaggaaggctggctagggctgcggcgccccgcgggggctctgcccgcgtggcgctttgcgcgtggggcgcggggcacgtgcgcgtgtgcgcgtggagcgcggggtgtgtgcccgcgccgtgccccccgcgtgctgcctggcgtgagtcaccgcggggctcgcctttataaccgccgccaggctcgcagctccgcagagcagcccggcc

SEQ ID NO: 18 - DNA, 188 bp, Mus musculus
gcggcgccccgcgggggctctgcccgcgtggcgctttgcgcgtggggcgcggggcacgtgcgcgtgtgcgcgtggagcgcggggtgtgtgcccgcgccgtgccccccgcgtgctgcctggcgtgagtcaccgcggggctcgcctttataaccgccgccaggctcgcagctccgcagagcagcccggcc

SEQ ID NO: 19 - DNA, 72 bp, Artificial Sequence
  promoter
agccagcacgtgtatgcgggaacgcacgtgagagaagctgagcacgtgaggctcgcggggcacgtgcgcgtg

SEQ ID NO: 20 - protein, 81 aa, Artificial Sequence
  chimera
AVITGACERDVQCGAGTCCAISLWLRGLRMCTPLGREGEECHPGSHKVPFFGRRMHHTCPCLPGLACLRTSFNRFICLAQK

SEQ ID NO: 21 - protein, 86 aa, Artificial Sequence
  chimera
AVITGACDKDSQCGGGMCCAVSIWVKSIRICTPMGKLGDSCHPLTRKVPFFRKRKHHTCPCLPNLLCSRFPDGRYRCSMDLKNINF

SEQ ID NO: 22 - DNA, 1204 bp, Artificial Sequence
  synthetic probe
cgccatgaggagcctgtgctgcgccccactcctgctcctcctgctgctgccgccgctgctgctcacgccccgcgtcggggacgccgccgtgatcaccggggcttgtgacaaggactcccaatgtggtggaggcatgtgctgtgctgtcagtatctgggttaagagcataaggatttgcacacctatgggcaaactgggagacagctgccatccactgactcgtaaagttccatttgttgggcggaggatgcatcacacttgcccatgtctgccaggcttggcctgtttacggacttcatttaaccgatttatttgtttagcccgaaagtaatcgctttaaagtagaaaccaaatgtgaatagccacatcttatctgtaaagtcttacttgtgattgtgccaaacaaaaaatgtgccagaaagaaatgcttttgcttcctcaactttccaagtaacttttttatctttgagttttaaatgattttttttttaatcgggaattttacttttggatagaaatataaagtgtaaggcattgtggaactggttctcatttccctgtttgtgttttggtttggtttggcttttttcttaaatgtcaaaaacatacccattttcacaaaaatgaggaaaataggaatttgatattttgttagagaaactttttttttcctcaccatcccaagccccatttgtgccccgccacaccataccatacatacatacatacatacatacatacatacatacaacttttggtcccttgcctcttccacctcaaagaatttcaaggcccttaccttactttatttttctccatttctcttccctgctcttgcattttaaagtggtaggtttatctctttgagtttgatggcagaatcgctgatgggaatccagctttttgccggctatttaaatagtgaaaagagtttatatgtgaacttgacactccaaactcctctcatggcgtggacgctgggagtgctgccggacccttcctaaacctgtcactcaagaggacttcggctctgctgttgggctggtgtgtggacagaaggaatggaaagctaaattaatttagtccagatttctaggtttgggtttttctaaaaatgaaagattacgtttacttctttttctttttataaagtttttttttcttagtctcctacttagagatattctagaaaatgtcacttgaa

SEQ ID NO: 23 - DNA, 617 bp, Artificial Sequence
  synthetic probe
gaattcgcccttgtgcatcgccatgagcaacagcatgatcaacaccgtgtgctttgtgacagtcaagaacaacaccatgaagtacttcaagaagatgatgctgctgcactggcgtccctcccagcgggggagcaagtccagcgccgagcttgacctcagaaccaacggggtgcccgccacagaagaggtggactgtatcaggctgaagtgaccccctggtgtcacacaacagaaaaccccagtccagtactcaaagcatcacccaccatcaaccaagttcataggctgcatgggaagtgacatctgtgttcatgcctcccccatgccctcaagaagcccaacactgcaaagtcataacatacaatgacactagacatgaaccaaatcagctgacatttactgacatctgctcgacacctgctgtgtccacaatcccaacaaggagattagacacaaggagcagcgactgacatggactgaacacatactgtgtgcaaaccacaccaatgagatcagacagggacagcaggagctgacatttactcattacctactgtaatcaaaaacatttaaaaacataacaaaatggcagaagctattggagtccccaagatatc

SEQ ID NO: 24 - DNA, 762 bp, Artificial Sequence
  synthetic probe
ccttcaagtgaccatgagaggtgccacgctagtctcaatcatgttcctcctagtaactgtgtctgactgtgctgtgatcacaggggcctgtgagcgggatgtccagtgtggggcaggcacctgctgtgccatcagcctgtggcttcgagggctgcggatgtgtaccccgctggggcgggaaggcgaggagtgccatcctggcagccacaaggtccccttctttaggaaacgcaagcaccatacctgtccttgctcacccaacctgctgtgctccaggttcccagacggcaggtaccgctgctccatggacttgaagaacatcaatttttaggcgcttgccgggtctcaggatacccaccatccttttccccagcactgcctggatttttatttgtgccatgcaacccagctcctgtgactcttccagtccctacgctgactactttgatctctcttgcctagtacacacatatgcacacagggagacatacctcccatcatgacgtggtccccaggctggcctgacgatgtcccagcttggagctgtggtgtgagagatggccagcctggttcccttccctgcttaggctgccagagaggtggtaaatggcagagaggacattccccctcccctcccttcctgggcctgctctccttcctgggccctgtccctctccccacatgtaccccgcggtctgaattggacattcctggcacaggctcttgggtgcattgtgggcacaggctcttgggtgcattgaa

SEQ ID NO: 25 - DNA, 470 bp, Artificial Sequence
  synthetic probe
gatctgctcatagatgcataaaggaaagggtggcgagtctccggttagaagatgagtttagggttttggaaactccagaatcggaatatctttgtgatcctggaagtgctgaccatcttcccatgactgttcgcagccagcatgaatctgggcaatgggatcgttgcttctcagtctggttttcttctcacgagtgtgtagagctacagcgtggacttgtctttttcttatttaactctagagcagcacacctacaaggttttttaagtggcatgtgtgacatgttcccctccaaatatagtcaactccttattttgggcagcaaagtttattttgtatagttactttatttccctgaaagacacattacaaaaatctatttatttcttctattagatcacccaatagacttaccctacaatttaatcataatttaatcagaatgctcttggagagtaaagggcgaattc

(19) United States
(12) Patent Application Publication
(10) Pub. No.: US 2006/0172935 A1
(43) Pub. Date: Aug. 3, 2006

(54) Screening and therapeutic methods for treating circadian rhythm disorders

(76) Inventors: Qun-Yong Zhou, Irvine, CA (US); Clayton M. Bullock, West Hollywood, CA (US); Jerome Siegel, Northride, CA (US)

Correspondence Address: LISA A. HAILE, J.D., Ph.D.;DLA PIPER RUDNICK GRAY CARY US LLP., Suite 1100, 4365 Executive Drive, San Diego, CA, 92121-2133, US

(21) Appl. No.: 11/048,649

(22) Filed: Jan. 31, 2005

(51) Int. Cl. A61K 38/17

(52) U.S. Cl. 514/12

(57) ABSTRACT

The invention provides a method for screening for a compound for modulating circadian rhythm. The method involves (a) providing a compound that is a Prokineticin 2 (PK2) receptor antagonist or agonist; and (b) determining the ability of the compound to modulate one or more indicia of circadian rhythm function, wherein a compound that modulates one or more indicia of circadian rhythm function is identified as a compound for modulating circadian rhythm. The invention also provides a mouse PK2 receptor nucleic acid, polypeptide and related compositions. Further provided is a method for modulating circadian rhythm of an animal, which involves administering an effective amount of a PK2 receptor antagonist or agonist to an animal. Also provided is an isolated nucleic acid comprising a PK2 gene promoter operatively linked to a heterologous nucleotide sequence.

### BACKGROUND OF THE INVENTION

The daily rhythm of life is maintained by a circadian clock in organisms ranging from bacteria to humans. The time kept by a circadian clock enables the organism to respond physiologically and influences its behavior to daily environmental fluctuations. In humans, circadian rhythms help coordinate the timing of our internal bodily functions, including sleep, as well as our interactions with the external world. Virtually all known physiologic parameters and cellular activities are influenced by the body's circadian clock.

Only recently has the medical community, as well as the general public, become aware of the importance of circadian rhythms for human health, safety, performance and productivity. It is now recognized that physical and mental impairments are associated with night-work, which involves over 20% of the work force in industrialized countries. People who work late-night shifts often have problems falling asleep, staying asleep, or waking up. More than 25 million Americans have non-traditional work schedules, and it is estimated that 60 to 70 percent of these people have a chronic sleeping problem. In addition, many industries will employ increasing numbers of workers around the clock (for example, transportation, utilities, public safety, heavy manufacturing and many service industries).

Normal sleep depends on properly functioning circadian rhythm. In humans, obtaining less than the required number of hours of sleep, particularly over several nights, leads to a decreased ability to retain new information, impaired productivity, altered mood, lowered resistance to infection and an increased susceptibility to accidents. Sleep-related traffic accidents annually claim thousands of lives, and operator fatigue has also been shown to play a contributory role in airplane crashes and other catastrophic accidents.

The pharmaceutical industry is now investigating the importance of circadian rhythms for the timing of drug delivery and is interested in developing drugs that could affect the circadian clock of humans, as well as agricultural plants and animals. Numerous health problems, including some forms of depression as well as many sleep, neurological, cardiovascular and endocrine disorders, have recently been associated with circadian rhythm dysfunctions.

In addition, as the elderly continue to become a greater percentage of the population in the United States, as well as in the rest of the world, more and more circadian abnormalities are being observed in older people suffering from various sleep, mental and physical disorders.

Thus, there exists a need to identify molecules associated with circadian rhythm and methods for identifying new therapeutic agents that can be used to modulate circadian rhythm. The present invention satisfies these needs and provides related advantages as well.

### SUMMARY OF THE INVENTION

The invention provides a method for screening for a compound for modulating circadian rhythm. The method involves (a) providing a compound that is a Prokineticin 2 (PK2) receptor antagonist or agonist; and (b) determining the ability of the compound to modulate one or more indicia of circadian rhythm function, wherein a compound that modulates one or more indicia of circadian rhythm function is identified as a compound for modulating circadian rhythm.

In one embodiment, the provided PK2 receptor antagonist can be identified by contacting a receptor selected from PK2 receptor and PK1 receptor with one or more candidate compounds under conditions wherein PK2 promotes a predetermined signal and identifying a compound that reduces the predetermined signal.

In another embodiment, the provided PK2 receptor agonist can be identified by contacting a receptor selected from PK2 receptor and PK1 receptor with one or more candidate compounds under conditions wherein PK2 promotes a predetermined signal and identifying a compound that promotes the predetermined signal, for example, calcium ion mobilization.

In a further embodiment, the provided PK2 receptor antagonist can be identified by contacting a receptor selected from PK2 receptor and PK1 receptor with one or more candidate compounds in the presence of a receptor agonist under conditions wherein the agonist binds to the selected receptor and identifying a compound that reduces the binding.

In yet another embodiment, the provided PK2 receptor agonist can be identified by contacting a receptor selected from PK2 receptor and PK1 receptor with one or more candidate compounds under conditions wherein PK2 binds to a selected receptor and identifying a compound that binds to and activates the selected receptor.

The invention also provides a method for modulating circadian rhythm of an animal. The method involves administering to the animal an effective amount of a PK2 receptor antagonist or agonist, such as PK2, PK1 or a compound. Such treatment can be used to improve circadian rhythm disorders such as disorders of sleep/wakefulness rhythms and seasonal disorders. Exemplary conditions that can be beneficially treated with PK2 receptor agonist or PK2 receptor antagonist include non-24-hour sleep-wake syndrome, rapid time-zone change syndrome, work-shift syndrome, delayed phase sleep syndrome, advanced sleep phase syndrome, irregular sleep-wake pattern syndrome, syndrome associated with decreased amplitude. The method can also be used to suppress or enhance various circadian behaviors, including REM sleep and NREM sleep in an animal. In addition, seasonal disorders such as seasonal affective disorder can be beneficially treated with PK2 receptor agonist or PK2 receptor antagonist.

In a specific embodiment, the invention provides a method for modulating a circadian locomotor activity in a mammal. The method involves administering to the mammal a PK2 receptor agonist or antagonist. Such a method may be used to modulate a behavior associated with the dark part of a dark/light cycle. In some embodiments, the method results in suppressing a behavior associated with the dark part of the dark/light cycle. In particular embodiments, the method results in suppression of REM sleep, wheel-running behavior or both in nocturnal mammals, such as rats and mice.

The invention provides another method for identifying a compound for modulating circadian rhythm. The method involves (a) contacting an isolated nucleic acid comprising a PK2 gene promoter operatively linked to a reporter nucleic acid with one or more candidate compounds under conditions wherein the reporter nucleic acid produces a predetermined signal in response to PK2 gene promoter activation; (b) identifying a compound that alters production of the signal; (c) providing the compound; and (d) determining the ability of the compound to modulate one or more indicia of circadian rhythm function, wherein a compound that modulates one or more indicia of circadian rhythm function is identified as a compound for modulating circadian rhythm.

The invention also provides compositions containing detectably labeled PK2 and an isolated mouse PK2 receptor.

Also provided by the invention is an isolated nucleic acid comprising a PK2 gene promoter operatively linked to a heterologous nucleotide sequence, which can be, for example, a reporter nucleotide sequence. In one embodiment, the invention provides a method for using the PK2 gene promoter for light-inducible expression of a nucleic acid molecule in an animal.

### BRIEF DESCRIPTION OF THE DRAWINGS

FIG. 1 shows rhythmic expression of PK2 mRNA in SCN. FIG. 1a shows PK2 mRNA expression in coronal sections (20 μm) of the mouse brain. FIG. 1b shows temporal profiles of PK2 mRNA in the suprachiasmatic nucleus (SCN). FIG. 1c shows representative images of PK2 mRNA expression shown in FIG. 1b.

FIG. 2 shows in vitro transcription analyses of mouse PK2 gene. FIG. 2a shows the boundaries of E-boxes (E) and cAMP-responsive element (CRE) within the 5′-flanking region of the mouse PK2 gene. FIG. 2b shows transcriptional activation of luciferase reporter containing 2.8 kb (PK2.8-Luc) and 200 bp (PKO.2-Luc) of the 5′-flanking region of mouse PK2 gene. FIG. 2c shows transcriptional activation of SV40-driven luciferase reporter (pGL3-Promoter) containing 72 bp of all four E-boxes and their immediate flanking sequences linked together (PK4E-Luc). FIG. 2d shows inhibition of CLOCK:BMAL1-mediated transcription from PK2.8-Luc by mPers and mCrys. FIG. 2e shows the positive effect of PK2 receptor activation on CLOCK:BMAL1-mediated transcription of the PK2 gene.

FIG. 3 shows rhythmic expression of PK2 mRNA in Clock-deficient (Clk/Clk) or Cryptochrome-deficient (Cry/Cry) mice. FIG. 3a shows temporal profiles of PK2 mRNA in SCN of Clk/Clk mice. FIG. 3b shows the expression of PK2 mRNA in SCN of Cry/Cry mice at CT6 and CT18.

FIG. 4 shows that PK2 rhythm in SCN responds to light entrainment when animals are exposed to a 15-min light pulse at CT14 (FIG. 4a) or CT23 (FIG. 4b). FIG. 4c shows a shift PK2 rhythm in SCN in response to light pulses. FIG. 4d shows quantification of phase shifts shown in FIG. 4c.

FIG. 5 shows expression of PK2 receptor (PKR2) mRNA in mouse brain. FIGS. 5a-e show autoradiographic images of PKR2 mRNA in lateral septum (LS), paraventricular thalamic (PVT) and hypothalamic nucleus (PVN), suprachiasmatic nucleus (SCN), paratenial nucleus (PT), paracentral nucleus (PC), lateral habenula (LHb), dorsal medial hypothalamic nucleus (DMH) and arcuate nucleus (Arc). FIGS. 5f-j show dark field microscopic images of PKR2 mRNA from the boxed regions in top panel (a-e). FIGS. 5k and 51 show microscope images of PK2. FIGS. 5m and 5n show PKR2 mRNA in SCN at high magnification.

FIG. 6 shows PK2 receptor (PKR2) mRNA expression in mouse brain. FIG. 6a shows PKR2 mRNA expression in the paracentral nucleus (PC), paraventricular thalamic nuclei (PVT), paraventricular hypothalamic nuclei dorsal cap (PaDC), and suprachiasmatic nucleus (SCN). FIG. 6b shows PKR2 mRNA expression in the lateral habenula (LHb), lateral globus pallidus (LGP), amygdala (Amg), paraventricular thalamic nuclei (PVT), dorsal medial hypothalamic nucleus (DMH) and arcuate nucleus (Arc). FIGS. 6c, d, e and f depict dark field microscopic images of the PKR2 mRNA from the boxed regions in FIGS. 6a and b.

FIG. 7 shows PK2 mRNA expression in the SCN in response to abrupt shifts of light/dark cycles. FIG. 7a shows experimental design. FIGS. 7b and c show PK2 mRNA expression in normal light-dark cycles.

FIG. 8 shows temporal profiles of PK2 mRNA in the SCN in response to abrupt shifts of light/dark cycle.

FIG. 9 shows the effect of intracerebroventricular delivery of recombinant human PK2 on wheel-running. FIGS. 9a and 9b show representative actogram of rats injected with PK2 (a) or saline (b) at CT14. FIG. 9c shows quantification of night and day locomotor activity following delivery of PK2 (n=7) or saline (n=6).

FIG. 10 shows a comparison of the amino acid sequences of human PK2s (SEQ ID NOS:5 and 6), mouse PK2s (SEQ ID NOS:7 and 8), human PK1 (SEQ ID NO:9), mouse PK1 (SEQ ID NO:10), frog Bv8 (SEQ ID NO:11), toad Bv8 (SEQ ID NO:12), and snake MIT1 (SEQ ID NO:13).

FIG. 11 shows binding of iodinated PK2 to PK2 receptor.

FIG. 12 shows calcium mobilization induced by PK2 binding to PK2 receptor.

FIG. 13 shows the boundaries of E-boxes (E) and cAMP-responsive element (CRE) within the 5′-flanking region of the human PK2 gene.

FIG. 14 shows a temporal profile of PK2 mRNA expression in the SCN in response to abrupt shifting of light/dark cycles.

FIG. 15 shows molecular rhythms in the SCN of mCry1,2−/− mice under normal light/dark or constant dark conditions.

FIG. 16 shows that the light inducibility of PK2 mRNA in the SCN of Opn4−/− mice is reduced in comparison to wild type mice.

FIG. 17 shows the effect of photoperiod on PK2 mRNA rhythm in the SCN.

FIG. 18 shows PK2 mRNA Expression in rhesus monkey brain tissue.

FIG. 19 shows absence of signal with sense PK2 and sense PKR2 probes.

FIG. 20 shows expression of PK2 mRNA in rhesus monkey suprachiasmatic nucleus (SCN) at various time points.

FIG. 21 shows a comparison of PK2 oscillation in the SCN of monkey and mouse.

FIG. 22 shows that PK2 mRNA levels oscillate in the rhesus monkey SCN and reach peak levels during daytime hours; serum melatonin peaks during nighttime hours.

FIG. 23 shows Expression of PKR2 mRNA in rhesus monkey brain sections (anterior to posterior).

FIG. 24 shows expression of PKR2 mRNA in rhesus monkey brain sections (anterior to posterior).

FIG. 25 shows the unique co-expression of PKR2 and PK2 mRNA in SCN target regions of the rhesus monkey brain.

FIG. 26 shows expression of PK1 mRNA in rhesus monkey brain tissue.

FIG. 27 shows expression of PKR2 and PK1 mRNA in the superior colliculus.

FIG. 28 shows the effect of acute PK2 (3rd ventricle) delivered at ZT14 on wheel-running activity.

FIG. 29 shows actograms from the day before injection of PK2 to mice.

FIG. 30 shows actograms from the day of injection into the 3rd ventricle at ZT14.

FIG. 31 shows the effect of low dose of PK2 (0.5 μg, 3rd ventricle) administered at ZT2 on wheel-running activity.

FIG. 32 shows the effect of acute infusion of PK2 (5 μg) into the lateral ventricle at ZT14 on food intake.

FIG. 33 shows the effect of acute infusion of PK2 (5 μg) into the lateral ventricle at ZT2 on food intake.

FIG. 34 shows the effect of PK2 (5 μg) delivered via the lateral ventricle at ZT2 on wheel-running activity.

FIG. 35 shows actograms for rats from the day before injection.

FIG. 36 shows Actograms from the day of injection into the lateral ventricle at ZT2.

FIGS. 37A-D show the effect of PK2 injection on % total and % NREM sleep time after injection at ZT14.

FIGS. 38A-D show the effect of PK2 on % REM sleep & % active wake time, after injection at ZT14.

FIGS. 39A-D show the effect of PK2 on % total and % NREM Sleep Time, injection at ZT2.

FIGS. 40A-D show the effect of PK2 on % REM sleep and % Active Wake Time, injection at ZT2.

FIG. 41 shows representative wheel-running actograms of Wild Type (WT) mice under alternating periods of light and dark (LD).

FIG. 42 shows representative wheel-running actograms of PK2−/− mice under LD.

FIG. 43 shows wheel-running counts and rhythmicity are reduced in PK2−/− mice in LD.

FIG. 44 shows wheel-running counts for WT mice in total darkness (DD).

FIG. 45 shows wheel-running counts for PK2−/− mice (DD).

FIG. 46 shows that wheel-running counts and rhythmicity are reduced in PK2−/− mice in DD.

FIG. 47 shows how food intake is reduced in PK2−/− mice compared with WT controls.

FIG. 48 shows how percent body fat is increased in PK2−/− compared to WT controls.

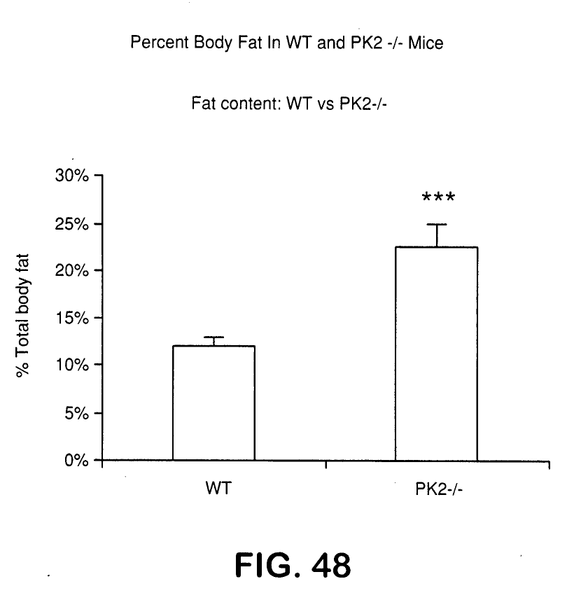

### DETAILED DESCRIPTION OF THE INVENTION

The present invention relates to the determination that expression of the prokineticin 2 (PK2) gene in the suprachiasmatic nucleus (SCN) oscillates in a circadian fashion, and to the observation that PK2 receptor activation modulates circadian rhythm in rats.

Circadian rhythms optimize biological efficiency by coordinating appropriate timing of physiological, endocrine and behavioral processes. Circadian rhythms are thought to contain at least three elements: a) input pathways(s) that relay environmental information to a circadian pacemaker (clock) b) a circadian pacemaker that generates the oscillation and c) output pathway(s) through which the pacemaker regulates various output rhythms. In mammals, the master circadian pacemaker resides in the suprachiasmatic nuclei (SCN) of the anterior hypothalamus (Klein et al., New York: Oxford Univ. Press. pp. 467 (1991)). Environmental light-dark cycles are input signals that entrain, or synchronize, the SCN clock to the 24-hr day via retinal-hypothalamic projections (Moore, Ann. Rev. Med. 48:253-66 (1997)). Synchronization of the cell-autonomous circadian clocks within the SCN leads to coordinated outputs (signaling pathways) that mediate the circadian regulation of physiology and behavior.

As disclosed herein, prokineticin 2 (PK2) is a SCN output molecule that transmits circadian rhythm. This role for PK2 is supported by the observations that PK2 is expressed in an oscillatory manner in the SCN (see Example I); production and/or release of PK2 is regulated by core clock genes (see Example II and III); production or release of PK2 responds to light entrainment (see Example IV); PK2 receptor is expressed in the primary SCN output target areas (see Example V); administration of PK2 results in changes in circadian behavior (see Example VI); and PK2 rhythmic output in the SCN is regulated by light (see Example VIII).

In particular, the inventors have shown that expression of PK2 gene in the SCN oscillates in a circadian fashion under normal light/dark cycle as well as in constant darkness. The oscillation amplitude of PK2 mRNA in the SCN is very high, with peak levels (day time) and trough levels (night time) differing by at least 50-fold. This magnitude of PK2 oscillation in the SCN is higher than other known clock or clock-controlled genes, including mpers, mCrys, and Bmal (Cermakian et al., EMBO J. 20:3967-74 (2001); Albrecht et al., Cell 91:1055-64 (1997); Tei et al., Nature 389:512-16 (1997); Miyamoto and Sancar, Proc. Natl. Acad. Sci. USA 95:6097-102 (1998)); and Zylka et al., Neuron 20:1103-10 (1998)). The observed positive feedback of PK2 on its own transcription could contribute to the high oscillation magnitude of PK2 mRNA (see Example II). Both in vitro transcription assays and analyses of mutant mice deficient in Cryptochromes and Clock I (see Examples II and III, respectively) indicate that PK2 is a clock-controlled gene.

PK2 transcription is activated by CLOCK and BMAL1 heterodimers via E-box enhancers in the 5′-flanking region, and this transactivation is also inhibited by the members of the negative limb of the central clockwork, including mPER1, mPER2, mPER3, mCRY1, and mCRY2. The organization of E-box enhancers in 5′-flanking region and the oscillation profile of PK2 in the SCN are most similar to those of mper1 (Albrecht et al., Cell 91:1055-64 (1997); and Tei et al., Nature 389:512-16 (1997)). Light pulse experiments show that PK2 rhythm in the SCN responds to light entrainment (see Example IV) and that abrupt shifts of light/dark cycles and changes in photoperiod alter PK2 expression (see Examples VIII and IX). Like mPer1 and mPer2 (Albrecht et al., Cell 91:1055-64 (1997); Tei et al., Nature 389:512-16 (1997); and Zylka et al., Neuron 20:1103-10 (1998)), PK2 responds to light induction only during subjective night. Light pulses delivered during early or late night result in delay or advance of PK2 rhythm in SCN, respectively. These Light-induced changes in PK2 rhythm are consistent with the shifts of behavioral rhythm (Daan and Pittendrigh, J. Comp. Physiol. 106:253-266 (1976); and Roenneberg and Foster, Photochem. Photobiol. 66:549-561 (1997)).

PK2 administration to rats, as shown in Example VI, provides direct evidence that PK2 is an output molecule that transmits the locomotor activity rhythm of the SCN. Specifically, Example VI shows that ICV administration of PK2 during subjective night suppressed the high nocturnal wheel-running behavior. These behavioral studies are consistent with the distribution of PKR2 mRNA in major primary target areas of SCN output pathways (Klein et al., New York: Oxford Univ. Press. 467 pp. (1991); Moore, Ann. Rev. Med. 48:253-66 (1997); Sofroniew and Weindl, Amer. J. Anat. 153:391-429 (1978); Watts et al., J. Comp. Neurol. 258:204-29 (1987); Watts and Swanson, J. Comp. Neurol. 258:230-52 (1987); Leak and Moore, J. Comp. Neurol. 433:312-34 (2001); and Buijs, Prog. Brain Res. 111:229-40, (1996)).

PK2 is a polypeptide ligand for the G-protein coupled receptor PKR2. Binding of PK2 to this receptor results in receptor activation, which induces a cascade of molecular events that culminate in alterations of cellular function. Such alterations in cellular function lead to cellular, physiological, endocrine, behavioral, and other responses that can be observed in an animal treated with PK2. Therefore, a PK2 receptor agonist or antagonist can be provided to an animal to alter a PK2-induced response, for example, to beneficially modulate circadian rhythm.

Based on this determination of an important pharmacological role of PK2 and PK2 receptor in control of circadian rhythm, the present invention provides methods of screening to identify compounds that modulate circadian rhythm, for example by modulating PK2 expression or PK2 receptor activity. Exemplary compounds that modulate PK2 receptor activity are PK2 receptor antagonist and agonists. Compounds identified using the methods of the invention can be used to modulate circadian rhythm for the treatment of circadian rhythm disorders.

The invention provides a method for identifying a compound for modulating circadian rhythm. The method involves (a) providing a compound that is a Prokineticin 2 (PK2) receptor antagonist or agonist; and (b) determining the ability of the compound to modulate one or more indicia of circadian rhythm function, wherein a compound that modulates one or more indicia of circadian rhythm function is identified as a compound for modulating circadian rhythm.

The methods of the invention also can be used to identify compounds useful for modulating biological rhythmicity having a period other than a 24 hour period. For example, activation of a PK2 receptor can modulate a biological rhythm having a periodicity of less than 24 hours (ultradian rhythm) or greater than 24 hours (infardian rhythm). Exemplary ultradian rhythms are daydreaming, urination, and hunger. Exemplary infardian rhythms include the frequency of receptivity of female animals to male animals for mating.

As used herein, the term “circadian rhythm” is intended to mean the regular variation in physiologic and behavioral parameters that occur over the course of about 24 hours.

As used herein, the term “modulating” when used in reference to circadian rhythm is intended to mean altering a physiological function, endocrine function or behavior that is regulated by the circadian timing system of an animal, or altering a cellular function that exhibits circadian rhythmicity. Exemplary physiological functions regulated by the circadian timing system of an animal include body temperature, autonomic regulation, metabolism, and sleep-wake cycles. Exemplary metabolic functions include control of weight gain and loss, including increase or decrease in body weight and increase or decrease in percent body fat. Exemplary endocrine functions regulated by the circadian timing system of an animal include pineal melatonin secretion, ACTH-cortisol secretion, thyroid stimulating hormone secretion, growth hormone secretion, neuropeptide Y secretion, serotonin secretion, insulin-like growth factor type I secretion, adrenocorticotropic hormone secretion, prolactin secretion, gamma-aminobutyric acid secretion and catecholamine secretion. Exemplary behaviors regulated by the circadian timing system of an animal include movement (locomotor rhythm), mental alertness, memory, sensorimotor integration, feeding, REM sleep, NREM sleep and emotion. Exemplary cellular functions that exhibit circadian rhythmicity are neuron firing and transcriptional control of gene expression.

The methods of the invention for screening for a compound that modulates circadian rhythm involve providing a PK2 receptor antagonist or agonist. The PK2 receptor antagonist or agonist can be provided to a cell preparation, tissue, organ, organism or animal that has at least one observable index of circadian rhythm function and expresses a PK2 receptor. The ability of the PK2 receptor antagonist or agonist to modulate circadian rhythm can be tested in a variety of animal species that exhibit indicia of circadian rhythm function, as well as organs, tissues, and cells obtained from such animals, and cell preparations derived therefrom. The provided PK2 receptor antagonist or agonist can be a known PK2 receptor antagonist or agonist, such as PK2, PK1 or a PK2/PK1 chimera, or can be a compound identified as a PK2 receptor antagonist or agonist using in vitro screening methods described herein.

A variety of in vitro screening methods are useful for identifying a PK2 receptor antagonist or agonist to be provided in the methods of the invention for identifying a compound that modulates circadian rhythm. The ability of a compound to modulate PK2 receptor can be indicated, for example, by the ability of the compound to bind to and activate PK2 receptor, block agonist binding to PK2 receptor, promote a predetermined signal produced by a PK2 receptor, or reduce a predetermined signal produced by a PK2 receptor. Therefore, signaling and binding assays can be used to identify a PK2 receptor antagonist or agonist that is provided in the methods of the invention for identifying a compound that modulates circadian rhythm.

A signaling or binding assay used to identify a PK2 receptor antagonist or agonist can contain a PK2 receptor or a PK1 receptor. Because of the homology between PK2 and PK1 receptors, which have amino acid sequences that are about 85% identical, a PK2 receptor or PK1 receptor can be used in screening assays to identify a PK2 receptor agonist. Specifically, due to the homology between the PK1 receptor and PK2 receptor, a PK1 receptor agonist or antagonist is likely to also function as a PK2 receptor agonist or antagonist. Similarly, either PK1 or PK2 can function as an agonist in signaling and binding assay formats that employ a competitive agonist.

When a signaling assay is used to identify a PK2 receptor antagonist or agonist, the methods of the invention can involve contacting a PK1 receptor or PK2 receptor with one or more candidate compounds under conditions in which PK2 promotes a predetermined signal and identifying a compound that either decreases or increases the predetermined signal, respectively. When a binding assay is used to identify a PK2 receptor antagonist or agonist, the methods of the invention can involve contacting a PK1 receptor or PK2 receptor with one or more candidate compounds under conditions in which PK2 binds to the PK2 receptor and identifying a compound that either decreases binding of a PK2 receptor agonist to the PK1 receptor or PK2 receptor, or binds to and activates the PK1 receptor or PK2 receptor, respectively.

A PK2 receptor used in the screening methods of the invention can be, for example, a mouse or human PK2 receptor, including a recombinantly produced receptor or naturally occurring receptor present in a cell preparation. As used herein, the term “mouse PK2 receptor” is intended to mean a heptahelical membrane-spanning G-protein-coupled receptor comprising the amino acid sequence of mouse PK2 receptor, or a naturally-occurring or man-made minor modification thereof that binds to PK2 and signals through a G-protein coupled signal transduction pathway in response to PK2. A PK2 receptor also can bind to PK1 to induce PK2 receptor signaling. The invention provides a mouse PK2 receptor, which has the amino acid sequence referenced as SEQ ID NO:2, and is encoded by the nucleotide sequence referenced as SEQ ID NO:1. The invention also provides a screening composition containing a mouse PK2 receptor in the presence of PK1 or PK2.

Similarly, a PK1 receptor used in the screening methods of the invention can be, for example, a mouse or human PK1 receptor, including a recombinantly produced receptor or naturally occurring receptor present in a cell preparation. As used herein, the term “mouse PK1 receptor” is intended to mean a heptahelical membrane-spanning G-protein-coupled receptor comprising the amino acid sequence of mouse PK1 receptor, or a naturally-occurring or man-made minor modification thereof that binds to PK1 or PK2 and signals through a G-protein coupled signal transduction pathway in response to PK1 or PK2. An exemplary mouse PK1 receptor has the amino acid sequence referenced as SEQ ID NO:4, and is encoded by the nucleotide sequence referenced as SEQ ID NO:3.

A minor modification of the sequence referenced as SEQ ID NO:2 or 4 can have one or more additions, deletions, or substitutions of natural or non-natural amino acids relative to the native polypeptide sequence. Such a modification can be, for example, a conservative change, wherein a substituted amino acid has similar structural or chemical properties, for example, substitution of an apolar amino acid with another apolar amino acid (such as replacement of leucine with isoleucine). Such a modification can also be a nonconservative change, wherein a substituted amino acid has different but sufficiently similar structural or chemical properties so as to not adversely affect the desired biological activity, such as, replacement of an amino acid with an uncharged polar R group with an amino acid with an apolar R group (such as replacement of glycine with tryptophan). Further, a minor modification of the mouse PK2 receptor amino acid sequence referenced as SEQ ID NO:2 or 4 can be the substitution of an L-configuration amino acid with the corresponding D-configuration amino acid or with a non-natural amino acid.

In addition, a minor modification of SEQ ID NO:2 or 4 can be a chemical or enzymatic modification to the polypeptide, such as replacement of hydrogen by an alkyl, acyl, or amino group; esterification of a carboxyl group with a suitable alkyl or aryl moiety; alkylation of a hydroxyl group to form an ether derivative; phosphorylation or dephosphorylation of a serine, threonine or tyrosine residue; or N- or O-linked glycosylation.

Those skilled in the art can determine whether minor modifications to the native mouse PK2 receptor sequence or PK1 receptor sequence are advantageous. Such modifications can be made, for example, to enhance the stability, bioavailability or bioactivity of the mouse PK2 receptor or PK1 receptor. A modified mouse PK2 receptor or PK1 receptor polypeptide can be prepared, for example, by recombinant methods, by synthetic methods, by post-synthesis chemical or enzymatic methods, or by a combination of these methods, and tested for ability to bind PK2 or PK1 or signal through a G-protein coupled signal transduction pathway.

Those skilled in the art also can determine regions in a mouse PK2 receptor or PK1 receptor amino acid sequence that can be modified without abolishing PK2 binding or signaling through a G-protein coupled signal transduction pathway. Structural and sequence information can be used to determine the amino acid residues important for PK2 receptor or PK1 receptor activity. For example, comparisons of amino acid sequences of PK2 receptor or PK1 receptor sequences from different species can provide guidance in determining amino acid residues that can be altered without abolishing activity.

Further, a large number of published GPCR structure-function studies have indicated regions of GPCRs involved in ligand interaction, G-protein coupling and in forming transmembrane regions, and indicate regions of GPCRs tolerant to modification (see, for example, Burstein et al., J. Biol. Chem., 273(38):24322-7 (1998) and Burstein et al., Biochemistry, 37(12) 4052-8 (1998)). In addition, computer programs known in the art can be used to determine which amino acid residues of a GPCR, such as a mouse PK2 receptor, can be modified as described above without abolishing activity (see, for example, Eroshkin et al., Comput. Appl. Biosci. 9:491-497 (1993)).

As used herein, the term “PK2 receptor agonist” is intended to mean a compound that selectively promotes or enhances normal signal transduction through the PK2 receptor. A PK2 receptor agonist can act by any agonistic mechanism, such as by binding a PK2 receptor at the normal PK2 binding site, thereby promoting PK2 receptor signaling. A PK2 receptor agonist can also act, for example, by potentiating the binding activity of PK2 or signaling activity of PK2 receptor. A PK2 receptor agonist can also be a PK1 receptor agonist. As such, a PK1 receptor agonist can be tested for its ability to function as a PK2 receptor agonist using the screening methods described herein.

Specific examples of PK2 receptor agonists include human PK2 amino acid sequence SEQ ID NO:5 and 6; mouse PK2 amino acid sequences SEQ ID NOS:7 and 8, human PK1 amino acid sequence SEQ ID NO:9; mouse PK1 amino acid sequence SEQ ID NO:10; toad Bv8 amino acid sequence SEQ ID NO:12; frog Bv8 amino acid sequence SEQ ID NO:11, and snake MIT1 (SEQ ID NO:13). The major human PK2 amino acid sequence is that referenced as SEQ ID NO:5, while the PK2 amino acid sequence referenced as SEQ ID NO:6 is a tissue specific PK2 amino acid sequence expressed at least in testes. Similarly, the major mouse PK2 amino acid sequence is that referenced as SEQ ID NO:7, while the PK2 amino acid sequence referenced as SEQ ID NO:8 is a less commonly expressed PK2 amino acid sequence.

A PK2 receptor agonist can include a modification of PK2 or PK1 that is capable of binding to and activating a PK2 receptor. Such a modification can be, for example, one or more additions, deletions or substitutions compared with the recited amino acid sequence; one or more chemical or enzymatic modifications to the polypeptide; or substitution of one or more L-configuration amino acids with corresponding D-configuration amino acids.

A PK2 receptor agonist can include a chimeric polypeptide containing amino acid sequence encoded by both PK1 and PK2 genes. Exemplary PK2 receptor agonists include a chimeric polypeptide encoded by exons 1 and 2 of PK1 and exon 3 of PK2, referenced as SEQ ID NO:20, and a chimeric polypeptide encoded by exons 1 and 2 of PK2 and exon 3 of PK1, referenced as SEQ ID NO:21. PK2 receptor agonists also include other natural or synthetic cyclic peptides that activate signaling through the PK2 receptor at nanomolar concentrations.

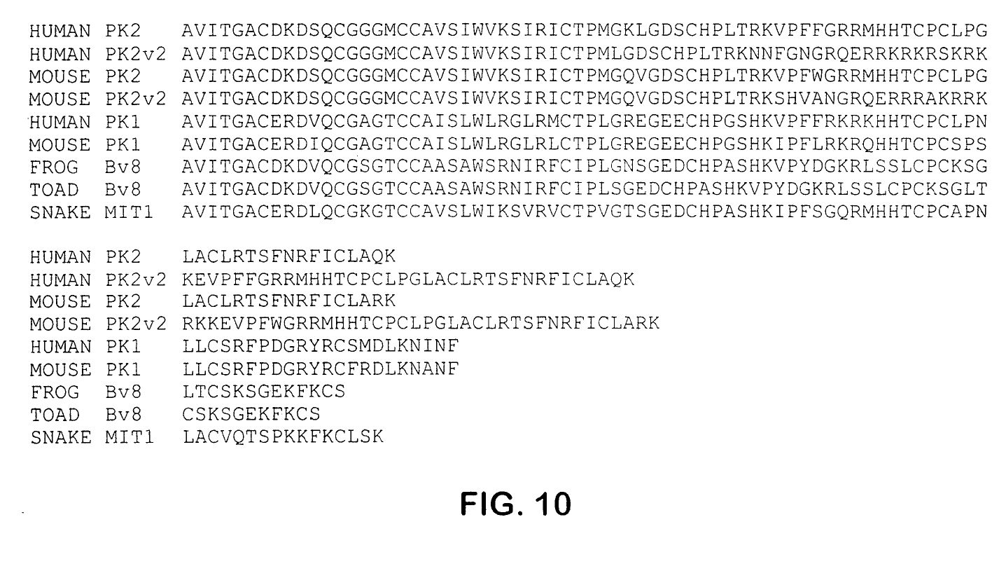

Guidance in modifying amino acid residues of a PK2 or PK1 while retaining activity can be provided by comparison with corresponding PK2 or PK1 sequences from mammalian or non-mammalian vertebrate species. FIG. 10 provides a comparison of amino acid sequences of PK1 from mouse and human, PK2 from mouse and human, and an ortholog representing PK1 and PK2 genes from frog and toad (Bv8) and snake (MIT1). It is well known in the art that evolutionarily conserved amino acid residues and domains are more likely to be important for maintaining biological activity than less well-conserved residues and domains. Thus, it would be expected that substituting a residue that is highly conserved among mammalian prokineticins and vertebrate orthologs, such as the N-terminal sequence, or any of the 10 conserved cysteines, would likely be detrimental to activity, whereas substitution of less highly conserved residues, such as the C-terminal residues, is likely to be tolerated.

As used herein, the term “PK2 receptor antagonist” is intended to mean a compound that selectively inhibits or decreases normal signal transduction through the PK2 receptor. A PK2 receptor antagonist can act by any antagonistic mechanism, such as by binding a PK2 receptor or PK2, thereby inhibiting binding between PK2 and PK2 receptor. A PK2 receptor antagonist can also inhibit binding between a specific or non-specific PK2 receptor agonist and PK2 receptor. Such a specific or non-specific PK2 receptor agonist can be, for example, a drug that produces unwanted side effects by promoting signaling through the PK2 receptor. A PK2 receptor antagonist can also act, for example, by inhibiting the binding activity of PK2 or signaling activity of PK2 receptor. For example, a PK2 receptor antagonist can act by altering the state of phosphorylation or glycosylation of PK2 receptor. A PK2 receptor antagonist can also be an inverse agonist, which decreases PK2 receptor signaling from a baseline amount of constitutive PK2 receptor signaling activity.

As used herein, the term “predetermined signal” is intended to mean a readout, detectable by any analytical means, that is a qualitative or quantitative indication of activation of G-protein-dependent signal transduction through PK2 receptor. Assays used to determine such qualitative or quantitative activation of G-protein-dependent signal transduction through PK2 receptor, are referred to below as “signaling assays.” G-proteins, or heterotrimeric GTP binding proteins, are signal transducing polypeptides having subunits designated Gα, Gβ and Gγ, that couple to seven-transmembrane cell surface receptors. G-proteins couple to such receptors to transduce a variety of extracellular stimuli, including light, neurotransmitters, hormones and odorants to various intracellular effector proteins. G-proteins are present in both eukaryotic and prokaryotic organisms, including mammals, other vertebrates, flies and yeast.

A signaling assay can be performed to determine whether a candidate compound is a PK2 receptor agonist or antagonist. In such an assay, a PK2 receptor is contacted with one or more candidate compounds under conditions wherein the PK2 receptor produces a predetermined signal in response to a PK2 agonist, such as PK2. In response to PK2 receptor activation, a predetermined signal can increase or a decrease from an unstimulated PK2 receptor baseline signal. A predetermined signal is an increasing signal, for example, when the amount of detected second messenger molecule is increased in response to PK2 receptor activation. A predetermined signal is a decreasing signal, for example, when the detected second messenger molecule is destroyed, for example, by hydrolysis, in response to PK2 receptor activation. A predetermined signal in response PK2 receptor activation can therefore be an increase in a predetermined signal that correlates with increased PK2 receptor activity, or a decrease in a predetermined signal that correlates with increased PK2 receptor activity. Accordingly, a PK2 receptor signaling assay of can be used to identify a PK2 receptor agonist that promotes production of a predetermined signal, whether the agonist promotes an increase in a predetermined signal that positively correlates with PK2 receptor activity, or a decrease in a predetermined signal that negatively correlates with PK2 receptor activity. Similarly, a signaling assay can be performed to determine whether a candidate compound is a PK2 receptor antagonist. In such a signaling assay, a PK2 receptor is contacted with one or more candidate compounds under conditions wherein the PK2 receptor produces a predetermined signal in response to a PK2 receptor agonist, such as PK2, and a compound is identified that reduces production of the predetermined signal.

Signaling through G proteins can lead to increased or decreased production or liberation of second messengers, including, for example, arachidonic acid, acetylcholine, diacylglycerol, cGMP, cAMP, inositol phosphate, such as inositol-1,4,5-trisphosphate, and ions, including Ca²⁺ ions; altered cell membrane potential; GTP hydrolysis; influx or efflux of amino acids; increased or decreased phosphorylation of intracellular proteins; or activation of transcription.

Various assays, including high throughput automated screening assays, to identify alterations in G-protein coupled signal transduction pathways are well known in the art. Various screening assay that measure Ca²⁺, cAMP, voltage changes and gene expression are reviewed, for example, in Gonzalez et al., Curr. Opin. in Biotech. 9:624-631 (1998); Jayawickreme et al., Curr. Opin. Biotech. 8:629-634 (1997); and Coward et al., Anal. Biochem. 270:2424-248 (1999). Yeast cell-based bioassays for high-throughput screening of drug targets for G-protein coupled receptors are described, for example, in Pausch, Trends in Biotech. 15:487-494 (1997). A variety of cell-based expression systems, including bacterial, yeast, baculovirus/insect systems and mammalian cells, useful for detecting G-protein coupled receptor agonists and antagonists are reviewed, for example, in Tate et al., Trends in Biotech. 14:426-430 (1996).

Assays to detect and measure G-protein-coupled signal transduction can involve first contacting a sample containing PK1 receptor or PK2 receptor, such as an isolated cell, membrane or artificial membrane, such as a liposome or micelle, with a detectable indicator. A detectable indicator can be any molecule that exhibits a detectable difference in a physical or chemical property in the presence of the substance being measured, such as a color change. Calcium indicators, pH indicators, and metal ion indicators, and assays for using these indicators to detect and measure selected signal transduction pathways are described, for example, in Haugland, Molecular Probes Handbook of Fluorescent Probes and Research Chemicals, Sets 20-23 and 25 (1992-94). For example, calcium indicators and their use are well known in the art, and include compounds like Fluo-3 AM, Fura-2, Indo-1, FURA RED, CALCIUM GREEN, CALCIUM ORANGE, CALCIUM CRIMSON, BTC, OREGON GREEN BAPTA, which are available from Molecular Probes, Inc., Eugene Oreg., and described, for example, in U.S. Pat. Nos. 5,453,517, 5,501,980 and 4,849,362.

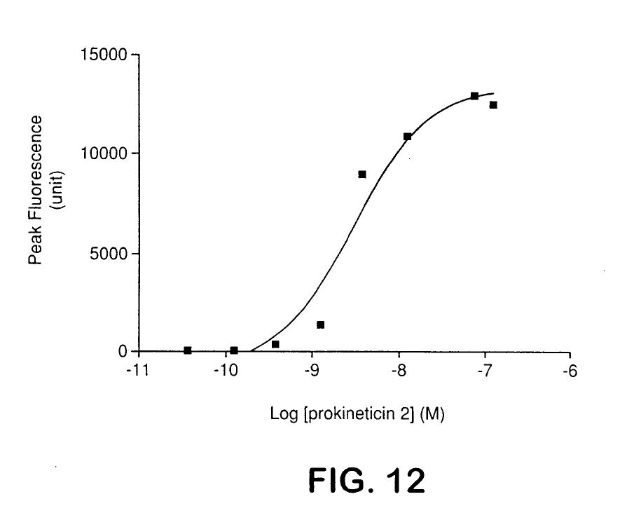

Signaling through PK2 receptor and PK1 receptor promotes intracellular calcium ion mobilization, suggesting that these receptors normally couple to Gαq-containing G proteins. Therefore, signaling through the PK2 receptor or PK1 receptor can be detected by any assay known in the art that detects intracellular calcium ion mobilization, including that described in Example VII. A calcium ion mobilization assay can be performed in the presence or absence of a PK1 or PK2. FIG. 12 shows the results of an exemplary PK2 receptor activation assay in which the predetermined signal is calcium ion mobilization. FIG. 12 indicates that the k_{d} for human PK2 activation of mouse PK2 receptor is 2.9 nM.

If desired, a predetermined signal other than Ca²⁺ influx can be used as the readout for PK2 receptor activation. The specificity of a G-protein for cell-surface receptors is determined by the C-terminal five amino acids of the Gα subunit. The nucleotide sequences and signal transduction pathways of different classes and subclasses of Gα subunits in a variety of eukaryotic and prokaryotic organisms are well known in the art. Thus, any convenient G-protein mediated signal transduction pathway can be assayed by preparing a chimeric Gα containing the C-terminal residues of a Gα that couples to PK2 receptor or PK1 receptor, such as Gαq, with the remainder of the protein corresponding to a Gα that couples to the signal transduction pathway it is desired to assay. Methods of recombinantly expressing chimeric Ga proteins are known in the art and are described, for example, in Conklin et al., Nature 363:274-276 (1993), Komatsuzaki et al., FEBS Letters 406:165-170 (1995), and Saito et al., Nature 400:265-269 (1999). Additionally, chimeric Gα proteins can be prepared by synthetic methods.

Another type of signaling assay involves determining changes in gene expression in response to a PK2 receptor or PK1 receptor agonist or antagonist. A variety of signal transduction pathways contribute to the regulation of transcription in animal cells by stimulating the interaction of transcription factors with genetic sequences termed response elements in the promoter regions of responsive genes. Assays for determining the interaction of transcription factors with promoter regions to stimulate gene expression are well known to those skilled in the art and are commercially available. As described herein, the PK2 promoter is activated in response to PK2 receptor activation (see Example II). Therefore, a variety of promoters, including a PK2 promoter, can be employed in gene expression assays to detect PK2 receptor or PK1 receptor activity. Exemplary gene expression assays are those that involve transducing cells with a promoter-reporter nucleic acid construct such that a readily detectable protein such as β-lactamase, luciferase, green fluorescent protein or galactosidase will be expressed in response to contacting PK2 receptor or PK1 receptor with an agonist, such as PK2, PK1 or a PK2/PK1 chimera. Compounds identified in such gene expression assays can act either at the level of the cell surface, by modulating the activity of a PK2 receptor, the activity of a component of the PK2 receptor signal cascade or the activity of factors that modulate transcription of a PK2-controlled gene.

An assay to identify compounds that function as PK2 receptor agonists or antagonists is performed under conditions in which contacting the receptor with a known PK2 receptor agonist would produce a predetermined signal. If desired, the assay can be performed in the presence of a known PK2 receptor agonist, such as a PK2, including those referenced as SEQ ID NOS:5, 6, 7 and 8, or a PK1, including those referenced as SEQ ID NOS:9 and 10, or a PK2/PK1 chimera, including those referenced as SEQ ID NOS:20 and 21. The agonist concentration can be within 10-fold of the EC₅₀. Thus, an agonist that competes with PK2, PK1 or a PK2/PK1 chimera, for signaling through the PK2 receptor, or indirectly potentiates the signaling activity of PK2, can be readily identified. Similarly, an agonist that competes with PK2, PK1 or a PK2/PK1 chimera for signaling through the PK1 receptor can be readily identified.

Likewise, an antagonist that prevents PK2, PK1 or a PK2/PK1 chimera from binding the PK2 receptor, or indirectly decreases the signaling activity of PK2 receptor, also can be identified. Similarly, an antagonist that prevents PK2, PK1 or a PK2/PK1 chimera from binding the PK1 receptor, or indirectly decreases the signaling activity of PK1 receptor, also can be identified. The candidate compound can be tested at a range of concentrations to establish the concentration where half-maximal signaling occurs; such a concentration is generally similar to the dissociation constant (Kd) for PK2 receptor binding.

A binding assay can be performed to identify compounds that are PK2 receptor agonists or antagonists. In such an assay, a PK2 receptor or PK1 receptor can be contacted with one or more candidate compounds under conditions in which PK2 binds to the selected receptor and a compound that binds to the selected receptor or that reduces binding of an agonist to selected receptor can be identified. Contemplated binding assays can involve detectably labeling a candidate compound, or competing an unlabeled candidate compound with a detectably labeled PK2 agonist, such as a PK2, PK1 or PK2/PK1 chimera. A detectable label can be, for example, a radioisotope, fluorochrome, ferromagnetic substance, or luminescent substance. Exemplary radiolabels useful for labeling compounds include ¹²⁵I, ¹⁴C and ³H. Methods of detectably labeling organic molecules, either by incorporating labeled amino acids into the compound during synthesis, or by derivatizing the compound after synthesis, are known in the art.

In order to determine whether a candidate compound decreases binding of detectably labeled PK2 to PK2 receptor, the amount of binding of a given amount of the detectably labeled PK2 is determined in the absence of the candidate compound. Generally the amount of detectably labeled PK2 will be less than its K_{d}, for example, 1/10 of its K_{d}. Under the same conditions, the amount of binding of the detectably labeled PK2, PK1 or PK2/PK1 chimera in the presence of the candidate compound is determined. A decrease in binding due to a candidate compound characterized as a PK2 receptor ligand is evidenced by at least 2-fold less, such as at least 10-fold to at least 100-fold less, such as at least 1000-fold less, binding of detectably labeled PK2, PK1 or PK2/PK1 chimera to PK2 receptor in the presence of the candidate compound than in the absence of the candidate compound.

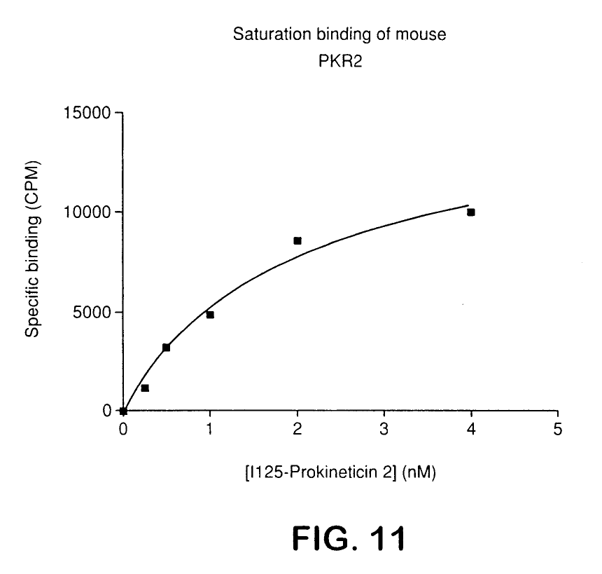

An exemplary assay for determining binding of detectably labeled PK2, PK1 or PK2/PK1 chimera to PK2 receptor or PK1 receptor is the radioligand filter binding assay described in Li et al. Molecular Pharmacology 59:692-698 (2001)). FIG. 11 shows the results of an exemplary PK2 receptor binding assay. FIG. 11 indicates that the Kd for ¹²⁵I-human PK2 binding to mouse PK2 receptor is about 1 nM. A variety of other low- and high-throughput assays suitable for detecting selective binding interactions between a receptor and a ligand are known in the art. Such assays include, for example, fluorescence correlation spectroscopy (FCS) and scintillation proximity assays (SPA) reviewed in Major, J. Receptor and Signal Transduction Res. 15:595-607 (1995); and in Sterrer et al., J. Receptor and Signal Transduction Res. 17:511-520 (1997)). Binding assays can be performed in any suitable assay format including, for example, cell preparations such as whole cells or membranes that contain PK2 receptor or PK1 receptor, or substantially purified PK2 receptor polypeptide or PK1 receptor, either in solution or bound to a solid support.

A detectably labeled PK2, PK1 and PK2/PK1 chimera can be useful in many of the in vitro assays described above. PK2, PK1 and PK2/PK1 chimeras can be derivatized with, or conjugated to, a moiety that is detectable by any analytical means. Such detectably labeled molecules useful in the assays disclosed herein generally retain their ability to bind PK2 receptor or PK1 receptor at subnanomolar concentrations. For example, a detectable moiety can be a radioisotope, fluorochrome, ferromagnetic substance, or luminescent substance. In one embodiment, the detectably labeled PK2, PK1 or PK2/PK1 chimera is radiolabeled. Exemplary radiolabels useful for labeling peptides include ¹²⁵I, ¹⁴C and ³H. Methods of detectably labeling peptides, either by incorporating labeled amino acids into the peptide during synthesis, or by derivatizing the peptide after synthesis, are known in the art. As described in Li et al. supra (2001), an exemplary detectably labeled PK2, PK1 or PK2/PK1 chimera is human PK2, radioiodinated at the core Tyr with ¹²⁵I, which binds membranes of cells transfected with PK1 receptor with an apparent K_{d} of 70 pM.

Assay methods for identifying compounds that selectively bind to or modulate signaling through a PK2 receptor generally involve comparison to a control. One type of a “control” is a preparation that is treated identically to the test preparation, except the control is not exposed to the candidate compound. Another type of “control” is a preparation that is similar to the test preparation, except that the control preparation does not express the receptor, or has been modified so as not to respond selectively to PK2 or PK1. In this situation, the response of the test preparation to a candidate compound is compared to the response (or lack of response) of the control preparation to the same compound under substantially the same reaction conditions.

A compound identified to be a PK2 receptor agonist or antagonist can be tested for its ability to modulate one or more indicia of circadian rhythm function, as described herein, below.

The invention provides a further method for identifying a compound useful for modulating circadian rhythm. The method involves (a) contacting an isolated nucleic acid comprising a PK2 gene promoter operatively linked to a reporter nucleic acid with one or more candidate compounds under conditions wherein the reporter nucleic acid produces a predetermined signal in response to PK2 gene promoter activation; (b) identifying a compound that alters production of the signal; (c) providing the compound, and (d) determining the ability of the compound to modulate one or more indicia of circadian rhythm function, wherein a compound that modulates one or more indicia of circadian rhythm function is identified as a compound useful for modulating circadian rhythm.

A compound identified to be a modulator of a PK2 gene promoter, such as a human PK2 gene promoter, is a potential therapeutically useful compound for modulating circadian rhythm. Example II demonstrates that transcription of mouse PK2 can be regulated by transcription factor binding to multiple E-box enhancers in the PK2 promoter. Specifically, CLOCK:BMAL1 heterodimer binding to a 2.8 kb region of the mouse PK2 promoter was demonstrated to increase PK2 transcription by 172-fold. Therefore, the level of PK2 in a cell can be modulated by activating or inhibiting PK2 gene expression from the PK2 gene promoter. For example, the activity of the PK2 promoter can be increased directly by adding a compound that binds to the PK2 promoter and induces PK2 gene expression. In addition, the activity of the PK2 promoter can be increased indirectly by increasing the amount of activating transcription factor, such as a CLOCK:BMAL1 heterodimer or their human orthologs, bound to the promoter, or the length of time that the activating transcription factor remains bound to the PK2 promoter, decreasing the amount of inhibiting transcription factor, such as mPER1, mPER2, mPER3, mCRY1 and mCRY1 or their human orthologs, or decreasing the length of time an inhibiting transcription factor remains bound to the PK2 promoter. Similarly, the activity of the PK2 promoter can be decreased directly by adding a compound that binds to the PK2 promoter and inhibits PK2 gene expression. PK2 promoter activity can be decreased indirectly for example, by decreasing the amount of activating transcription factor, decreasing the length of time an activating transcription factor remains bound to the PK2 promoter, increasing the amount of inhibiting transcription factor, or increasing the length of time an inhibiting transcription factor remains bound to the PK2 promoter.

Increasing or decreasing the amount of transcription factor, or homomultimeric or hetermultimeric transcription factor complex, can be achieved, for example, by increasing the expression or stability of a transcription factor(s) that enhances PK2 expression; by adding exogenous enhancing transcription factor(s) or mimetics thereof; decreasing the expression or stability of a transcription factor(s) that inhibits PK2 expression, and by adding exogenous inhibitory transcription factor(s) or mimetic thereof. Compounds capable of modulating the activity of a PK2 promoter can be identified, for example, by adding a candidate compound to a sample containing a PK2 promoter linked to a reporter nucleic acid, and measuring reporter nucleic acid expression. Compounds that regulate PK2 gene expression can be identified using such assays, and that modulate one or more indicia of circadian rhythm function, can be used for modulating circadian rhythm, for example, in an animal, including a human. This type of PK2 transcriptional activity assay also is useful for determining the activity of a PK2 receptor in response to a PK2 receptor antagonist or agonist, as described above.

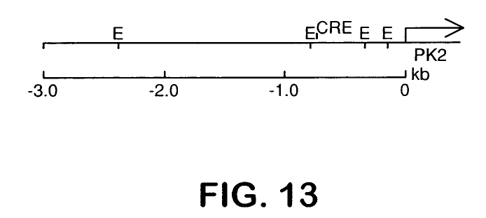

As disclosed herein, the promoter region of the human PK2 gene has been identified to contain several E-box enhancers and a cyclic AMP response element (CRE) (see, for example, FIG. 13, which shows the boundaries of the E-boxes (E) and CRE and the sequence of the promoter region, respectively). The screening method can employ an exemplary PK2 gene promoter comprising a nucleic acid sequence referenced as SEQ ID NO:14 (human PK2 promoter), SEQ ID NO:15 (human 2.8 kb PK2 promoter), SEQ ID NO:16 (mouse PK2 promoter), SEQ ID NO:17 (mouse 2.8 kb promoter), or SEQ ID NO:18 (mouse 188 bp PK2 promoter). Each of these PK2 gene promoters can be modulated to induce or reduce the expression of a PK2 gene in the methods of the invention.

As shown in Example VIII, expression of PK2 in the SCN is light regulated. Thus, the human PK2 gene promoter can be used in a method for light regulated expression of a nucleic acid molecule in an animal. In one embodiment, the invention provides a method for light regulated expression of a nucleic acid molecule in an animal that involves introducing into the animal an isolated nucleic acid molecule comprising a PK2 gene promoter operatively linked to a heterologous nucleotide sequence. A variety of methods are known in the art for introducing a nucleic acid molecule into a cell. Such methods include microinjection, electroporation, lipofection, calcium-phosphate mediated transfection, DEAE-Dextran-mediated transfection, polybrene- or polylysine-mediated transfection, and conjugation to an antibody, gramacidin S, artificial viral envelopes or other intracellular carriers such as TAT. For example, cells can be transformed by microinjection as described in Cibelli et al., Nat. Biotech. 16:642-646 (1998) or Lamb and Gearhart, Cur. Opin. Gen. Dev. 5:342-348 (1995); by lipofection as described in Choi (U.S. Pat. No. 6,069,010) or Lamb and Gearhart, Cur. Opin. Gen. Dev. 5:342-348 (1995); by electroporation as described in Current Protocols in Molecular Biology, John Wiley and Sons, pp 9.16.4-9.16.11 (2000) or Cibelli et al., Nat. Biotech. 16:642-646 (1998); or by fusion with yeast spheroplasts Lamb and Gearhart, Cur. Opin. Gen. Dev. 5:342-348 (1995).

A nucleic acid containing a PK2 gene promoter operatively linked to a heterologous nucleotide sequence can be delivered into a cell, either in vivo or in vitro using suitable vectors well-known in the art. Suitable vectors for delivering a nucleic acid to a mammalian cell, include viral vectors such as retroviral vectors, adenovirus, adeno-associated virus, lentivirus, herpesvirus, as well as non-viral vectors such as plasmid vectors.

Viral based systems provide the advantage of being able to introduce relatively high levels of the heterologous nucleic acid into a variety of cells. Suitable viral vectors for introducing a nucleic acid containing a PK2 gene promoter into a mammalian cell are well known in the art. These viral vectors include, for example, Herpes simplex virus vectors (Geller et al., Science, 241:1667-1669 (1988)); vaccinia virus vectors (Piccini et al., Meth. Enzymology, 153:545-563 (1987)); cytomegalovirus vectors (Mocarski et al., in Viral Vectors, Y. Gluzman and S. H. Hughes, Eds., Cold Spring Harbor Laboratory, Cold Spring Harbor, N.Y., 1988, pp. 78-84)); Moloney murine leukemia virus vectors (Danos et al., Proc. Natl. Acad. Sci. USA, 85:6460-6464 (1988); Blaese et al., Science, 270:475-479 (1995); Onodera et al., J. Virol., 72:1769-1774 (1998)); adenovirus vectors (Berkner, Biotechniques, 6:616-626 (1988); Cotten et al., Proc. Natl. Acad. Sci. USA, 89:6094-6098 (1992); Graham et al., Meth. Mol. Biol., 7:109-127 (1991); Li et al., Human Gene Therapy, 4:403-409 (1993); Zabner et al., Nature Genetics, 6:75-83 (1994)); adeno-associated virus vectors (Goldman et al., Human Gene Therapy, 10:2261-2268 (1997); Greelish et al., Nature Med., 5:439-443 (1999); Wang et al., Proc. Natl. Acad. Sci. USA, 96:3906-3910 (1999); Snyder et al., Nature Med., 5:64-70 (1999); Herzog et al., Nature Med., 5:56-63 (1999)); retrovirus vectors (Donahue et al., Nature Med., 4:181-186 (1998); Shackleford et al., Proc. Natl. Acad. Sci. USA, 85:9655-9659 (1988); U.S. Pat. Nos. 4,405,712, 4,650,764 and 5,252,479, and WIPO publications WO 92/07573, WO 90/06997, WO 89/05345, WO 92/05266 and WO 92/14829; and lentivirus vectors (Kafri et al., Nature Genetics, 17:314-317 (1997)). It is understood that both permanent and transient expression can be useful in a method of the invention.

A human promoter is useful in the methods for identifying a compound for modulating circadian rhythm because a compound so identified likely will function as a compound active in a human cell. However, a mouse PK2 promoter can also be used in the methods for identifying a compound for modulating circadian rhythm. A compound identified to regulate mouse PK2 promoter can modulate both mouse and human PK2 promoters because these PK2 promoters are structurally and functionally similar, can modulate a mouse PK2 promoter, or can modulate a PK2 promoter of another species.

As disclosed herein in Example I, a heterocomplex of CLOCK and BMAL1 functions as a positive regulator of mouse PK2 gene expression. Because of the similarity between the mouse and human PK2 promoter regions, a CLOCK:BMAL1 heterocomplex can be used to positively regulate human PK2 gene expression by binding to a human PK2 promoter. Therefore, a method of the invention can involve activating a human PK2 gene promoter by contacting the promoter with a heterocomplex of CLOCK and BMAL1.

When a method of the invention is employed to identify a compound that negatively regulates PK2 gene expression, a candidate compound is provided to an activated PK2 promoter. A PK2-promoter can be activated, for example, by addition of a CLOCK:BMAL1 heterocomplex or other transcription factor that positively regulates PK2 gene transcription, or by adding PK2 or a PK2 receptor agonist, depending on the assay format used. Specifically, a CLOCK:BMAL heterocomplex or other transcription factor can be used in a variety of assay formats, from minimal component systems containing only a reporter nucleic acid operatively linked to the PK2 promoter to systems containing intact cells having PK2 receptors that can be activated in. response to PK2. PK2 or another PK2 receptor agonist can be used in cell-based assay formats containing PK2 receptors that can be activated in response to PK2 and transmit a signal to the cell nucleus to regulate PK2 promoter activation.

When the method is employed to identify a compound that positively regulates PK2 gene expression, a candidate compound can be provided to an inactive PK2 promoter that has no bound transcription factors or has bound inhibitory transcription factor(s). The method can be used to identify a compound that binds directly to a PK2 promoter to increase promoter activity; a compound that binds to an inhibitory transcription factor to modulate its binding to a PK2 promoter, or a compound that functions by any other mechanism that results in an increase in PK2 gene expression.

A variety of nucleic acid molecules can be operatively linked to a PK2 gene promoter. As used herein, the term “operatively linked” is intended to mean that the nucleic acid molecule is positioned with respect to a promoter, such as a mouse or human PK2 promoter, in such a manner that the promoter will direct the transcription of RNA using the nucleic acid molecule as a template.

Methods for operatively linking a nucleic acid to a heterologous promoter are well known in the art and include, for example, cloning the nucleic acid into a vector containing the desired promoter, or appending the promoter to a nucleic acid sequence using PCR. A nucleic acid molecule operatively linked to a promoter of RNA transcription can be used to express reporter nucleic acid transcripts and polypeptides in a desired host cell or in vitro transcription-translation system. Exemplary reporter nucleic acids include luciferase, β-lactamase, β-glucuronidase, green fluorescent protein, blue fluorescent protein, chloramphenicol acetyltransferase and β-galactosidase.

As described above, circadian rhythm in an animal can be altered by modulating PK2 receptor activity by providing a PK2 receptor agonist or antagonist. A PK2 receptor agonist or antagonist can have a two-prong effect in altering a PK2-mediated biological response by modulating both PK2 receptor activity and PK2 gene expression, because as shown in Example II, PK2 receptor activation results in increased CLOCK:BMAL1-mediated PK2 gene expression. Specifically, CLOCK:BMAL1-mediated activation of PK2 gene transcription is regulated by PK2 receptor activity such that PK2 receptor activation increases transcription of the PK2 gene.

The level of PK2 expressed in cells of the SCN is under control of autoregulatory transcriptional and translational feedback loops that have both positive and negative elements. Positive transcriptional elements include two basic helix-loop-helix, PAS domain-containing transcription factors, CLOCK and BMAL1, which heterodimerize and drive the transcription of three Period genes (mPer1, mPer2, and mPer3) and two Cryptochrome genes (mCry1 and mCry2) by binding to their respective CACGTG E-box enhancers (Gekakis et al., Science 280:1564-69 (1998); Hogenesch et al., Proc. Natl. Acad. Sci. USA 95:5474-79 (1998)). The mPER and mCRY proteins act as negative components of the feedback loop, with mCRY proteins playing dominant inhibitory roles (Kume et al., Cell 98:193-205 (1999)). The amounts or activities of molecular components of these autoregulatory transcriptional and translational feedback loops can be altered to modulate circadian rhythm in an animal.

Methods for screening for a compound that modulates circadian rhythm, including a compound that is a PK2 receptor antagonist or agonist and a compound that modulates PK2 promoter activity, can involve contacting a PK2 receptor or PK2 promoter with one or more candidate compounds. A candidate compound can be a naturally occurring macromolecule, such as a peptide, nucleic acid, carbohydrate, lipid, or any combination thereof. A candidate compound also can be a partially or completely synthetic derivative, analog or mimetic of such a macromolecule, or a small organic or inorganic molecule prepared partly or completely by combinatorial chemistry methods.

A candidate compound further can be an antibody, including a monoclonal, humanized and chimeric antibodies, and functional fragments of an antibody includes chimeric, bifunctional, humanized and single chain antibodies (scFv), variable region fragments (Fv or Fd), Fab and F(ab)₂. An antibody can be naturally occurring or non-naturally occurring.

A candidate compound that is a nucleic acid can include, for example, an anti-sense nucleotide sequence, an RNA molecule, or an aptamer sequence. An anti-sense nucleotide sequence can bind to a nucleotide sequence within a cell and modulate the level of expression of a PK2 gene or PK2 receptor gene, or modulate expression of another gene that controls the expression of PK2 or PK2 receptor. Similarly, an RNA molecule, such as a catalytic ribozyme, can bind to and alter the expression of a PK2 gene, PK2 receptor gene, or other gene that controls the expression of PK2 or PK2 receptor. An aptamer is a nucleic acid sequence that has a three dimensional structure capable of binding to a molecular target, such as a PK2 or PK2 receptor (Jayasena, S. D. Clinical Chemistry 45:9, 1628-1650 (1999)). As such, an aptamer can serve as a PK2 receptor agonist or antagonist.

A candidate compound that is a nucleic acid also can be a double-stranded RNA molecule for use in RNA interference methods. RNA interference (RNAi) is a process of sequence-specific gene silencing by post-transcriptional RNA degradation, which is initiated by double-stranded RNA (dsRNA) homologous in sequence to the silenced gene. A suitable double-stranded RNA (dsRNA) for RNAi contains sense and antisense strands of about 21 contiguous nucleotides corresponding to the gene to be targeted that form 19 RNA base pairs, leaving overhangs of two nucleotides at each _{3′} end (Elbashir et al., Nature 411:494-498 (2001); Bass, Nature 411:428-429 (2001); Zamore, Nat. Struct. Biol. 8:746-750 (2001)). dsRNAs of about 25-30 nucleotides have also been used successfully for RNAi (Karabinos et al., Proc. Natl. Acad. Sci. 98:7863-7868 (2001). dsRNA can be synthesized in vitro and introduced into a cell by methods known in the art.

A candidate compound can be a peptidomimetic of an amino acid sequence of a PK2 polypeptide, such as any of SEQ ID NOS:5-14. Methods of rationally designing peptidomimetics of peptides, including neuropeptides, are known in the art. For example, the rational design of three peptidomimetics based on the sulfated 8-mer peptide CCK26-33, and of two peptidomimetics based on the 11-mer peptide Substance P, and related peptidomimetic design principles, are described in Horwell, Trends Biotechnol. 13:132-134 (1995). If desired in a particular assay format, a candidate compound can be detectably labeled or attached to a solid support.

The methods of the invention for screening for a compound that modulates circadian rhythm function can involve testing candidate compounds. The number of different candidate compounds to screen in a particular assay can be determined by those skilled in the art, and can be 2 or more, such as 5, 10, 15, 20, 50 or 100 or more different compounds. For certain applications, such as when a library of random compounds is to be screened, and for automated procedures, it may be desirable to screen 10³ or more compounds, such as 10⁵ or more compounds, including 10⁷ or more compounds.

Compounds for screening can be contained within large libraries of compounds, such as when high-throughput in vitro screening formats are used. Methods for producing large libraries of chemical compounds, including simple or complex organic molecules, metal-containing compounds, carbohydrates, peptides, proteins, peptidomimetics, glycoproteins, lipoproteins, nucleic acids, antibodies, and the like, are well known in the art and are described, for example, in Huse, U.S. Pat. No. 5,264,563; Francis et al., Curr. Opin. Chem. Biol. 2:422-428 (1998); Tietze et al., Curr. Biol., 2:363-371 (1998); Sofia, Mol. Divers. 3:75-94 (1998); Eichler et al., Med. Res. Rev. 15:481-496 (1995); and the like. Libraries containing large numbers of natural and synthetic compounds also can be obtained from commercial sources.

Compounds can be screened individually or in pools of a few, tens or hundreds of compounds. Therefore, a library of compounds can be screened sequentially, in a multi-sample format, in which each sample receives one compound, or multiplexed format, in which each sample receives more than one compound.

For the in vitro screening methods, a PK2 receptor or PK2 promoter can be contained in a cell preparation. As used herein, the term “cell preparation” is intended to mean a sample containing an isolated cell, which can be a cell contained in an organ, tissue, or cell culture and which contains a naturally occurring PK2 receptor. A cell preparation can contain intact, broken, solubilized, homogenized, or fractionated cells in the presence of a wide variety of components, such as buffers, salts and detergents, so long as PK2 receptor is capable of binding to PK2 and becoming activated in response to PK2 binding. A cell preparation also can be a cell line that expresses PK2 receptor. A cell line that expresses PK2 receptor can be identified by methods known in the art, such as competitive binding assays. An exemplary cell line that expresses PK2 receptor is the melanoma cell line M2A7 (available from American Type Culture Collection as ATCC CRL-2500). Other cell lines that express PK2 receptor include M2 melanoma cells (Cunningham et al., Science 255;325-327 (1992)) and RC-4B/C pituitary tumor cells (ATCC CRL-1903). A cell preparation also can include cells that recombinantly express PK2 receptor. A cell preparation can be obtained from a variety of animals, including, for example, humans, non-human primates, rats and mice. A human cell preparation, for example, is a sample containing an isolated human cell, which can be a cell contained in an organ, tissue, or cell culture and which contains a naturally occurring human PK2 receptor.

A cell preparation can be characterized as having circadian rhythmicity, although this feature is not required when the cell preparation is used in screening methods to determining if a compound is a PK2 receptor antagonist or agonist. As described in Li et al. Molecular Pharmacology 59:692-698 (2001)., human PK2 receptor is expressed in a variety of human tissues, including brain, heart, skeletal muscle, stomach and placenta. Therefore, any of these organs and other organs that express PK2 receptor can be a source for preparing a cell preparation for use in the screening methods of the invention.

A cell preparation having the characteristic of circadian rhythmicity can be advantageously used in in vitro assays to determine if a candidate compound or PK2 receptor agonist or antagonist modulates circadian rhythm function. Exemplary cells having circadian rhythmicity are SCN neurons, which retain a rhythmic firing pattern when dispersed in culture (Earnest et al., Science 283:693-695 (1999)).

A cell preparation also can include cells that recombinantly express PK2 receptor. Where it is desired to increase the PK2 receptor concentration, or to express PK2 receptor in host cells where it is not normally expressed, including mammalian, yeast and bacterial cells, the PK2 receptor can be recombinantly expressed. For example, a recombinantly expressed mouse or human PK2 receptor can be used in the methods of the invention.

Recombinant expression is advantageous in providing a higher level of expression of the polypeptide than is found endogenously, and also allows expression in cells or extracts in which the polypeptide is not normally found. A recombinant nucleic acid expression construct generally will contain a constitutive or inducible promoter of RNA transcription appropriate for the host cell or transcription-translation system, operatively linked to a nucleotide sequence that encodes the polypeptide of interest. The expression construct can be DNA or RNA, and optionally can be contained in a vector, such as a plasmid or viral vector. Based on knowledge of the nucleic acid sequence encoding PK2 receptor, one skilled in the art can recombinantly express desired levels of a biologically active PK2 receptor polypeptide using routine laboratory methods, described, for example, in standard molecular biology technical manuals, such as Sambrook et al., Molecular Cloning: A Laboratory Manual, Cold Spring Harbor Laboratory, New York (1992) and Ansubel et al., Current Protocols in Molecular Biology, John Wiley and Sons, Baltimore, Md. (1998).

Exemplary host cells that can be used to express recombinant PK2 receptor include isolated mammalian primary cells; established mammalian cell lines, such as COS, CHO, HeLa, NIH3T3, HEK 293-T and PC12; amphibian cells, such as Xenopus embryos and oocytes; and other vertebrate cells. Exemplary host cells also include insect cells (e.g. Drosophila), yeast cells (e.g. S. cerevisiae, S. pombe, or Pichia pastoris) and prokaryotic cells (e.g. E. coli), engineered to recombinantly express PK2 receptor. Additionally, recombinant PK2 receptor can be expressed in extracts that support transcription and translation, such as reticulocyte lysates and wheat germ extracts.

An appropriate assay for establishing that an isolated cell expresses PK2 receptor can be determined by those skilled in the art. Such an assay can involve, for example, analysis of expression of PK2 receptor nucleic acid or expression of PK2 receptor polypeptide by methods known in the art. Assays for determining expression of PK2 receptor mRNA include, for example, Northern blots, RT-PCR or in situ hybridization analysis. Such methods are described, for example, in standard molecular biology manuals such as Sambrook et al., supra, (1992) and Ansubel et al., supra, (1998). Assays for determining expression of PK2 receptor protein include, for example, immunoblot analysis, immunoprecipitation, immunofluorescence or immunohistochemistry, using antibodies specific for PK2 receptor.

A polypeptide of the invention, or one prepared for use in the methods of the invention, can be isolated from the cellular components with which they are normally associated using a variety of well-known methods, for example, precipitation, gel filtration, ion-exchange, reverse-phase and affinity chromatography, and the like. Other well known methods are described in Deutscher et al., Guide to Protein Purification: Methods in Enzymology Vol 182, Academic Press (1990). The methods and conditions for biochemical purification of a polypeptide can be selected by those skilled in the art, and purification monitored using a suitable immunological assay, functional assay or other biochemical assay.

The mouse PK2 receptor of the invention, as well as PK2, other PK2 receptors and candidate compounds used in the methods of the invention, can be recombinantly expressed, either transiently or stably, in a variety of host cells well known in the art (see, for example, Li et al., supra (2001)). Similarly, methods of recombinantly expressing chimeric Ga proteins, and their use in G-protein signaling assays, are known in the art and are described, for example, in, and Saito et al., Nature 400:265-269 (1999), and Coward et al., Anal. Biochem. 270:2424-248 (1999)). Such methods are applicable also to recombinantly producing a PK2 receptor agonist or antagonist that is a peptide or polypeptide.

Recombinant expression of polypeptides containing multiple cysteine residues often results in the incorrect formation of inter- and intra-molecular disulfide bonds, which leads to the production of inactive, aggregated bacterial proteins. As described in Li et al., supra (2001), these problems can be overcome using conditions that minimize protein aggregation during refolding of the expressed polypeptide. Exemplary conditions that minimize protein aggregation are described in the Example, and differ from conventional conditions for preparing recombinant protein by including one or more of the following refolding conditions: 1) keeping protein concentration low (e.g. about 100 μg/ml); 2) dialyzing, rather than diluting, the peptides to remove denaturing agent; 3) omitting oxidants from buffers; 4) maintaining high concentrations of urea in all buffers; 5) maintaining high concentrations of glycerol (e.g. at least about 10%) in buffers; and 6) keeping peptides and buffers at low temperature (e.g. about 4° C.). Of these conditions, it is contemplated that low protein concentration (for example, less than about 250 μg/ml, for example less than 200 μg/ml, 150 μg/ml, 100 μg/ml, or 50 μg/ml) and high urea concentration (e.g. at least about 1.5M, such as about 2M, 4M, 6M, 8M or higher) are the most important factors in successful refolding of active prokineticins.

It is expected that the same or similar conditions as those described herein can be used to recombinantly express and refold other polypeptides containing multiple cysteines, including other molecules that bind to and activate the PK2 receptor, so as to isolate a biologically active polypeptide.

Polypeptides and peptides of the invention, or prepared for used in the methods of the invention, including fragments and polypeptides having modifications, also can be generated by chemical synthesis. A variety of chemistries and instrumentation, included automated systems, such as Applied Biosystems, Inc. Model 430A or 431A, are well-suited for preparing peptides and polypeptides.

Antibodies or antibody fragments that are PK2 receptor agonist or antagonists or candidate compounds can be prepared using a variety of methods, including well-known methods for generating polyclonal and monoclonal antibodies in mice, rats, sheep and rabbits (see, for example, Harlow and Lane, Antibodies: A Laboratory Manual, Cold Spring Harbor Laboratory Press (1988)). Methods for generating chimeric, humanized, CDRS grafted, and reshaped antibodies are also well known to those skilled in the art (Huse et al., Science 246:1275-1281 (1989); Winter and Harris, Immunol. Today 14:243-246 (1993); Ward et al. Nature 341:544-546 (1989); Harlow and Lane, supra (1988); Hilyard et al., Protein Engineering: A practical approach (IRL Press 1992); Borrabeck, Antibody Engineering, 2d ed. (Oxford University Press (1995)). Fully human antibodies can also be generated using phage libraries and can be obtained from transgenic mice.

Upon identifying a compound that is a PK2 receptor agonist or antagonist, for example, using the screening methods described herein, such a compound can be tested to determine its effect on circadian rhythm. The effect of a PK2 receptor antagonist or agonist on a circadian rhythm that is maintained in a cell preparation, tissue or organ, such as neuronal firing and gene transcription, is predictive of the effect of the antagonist or agonist on a circadian rhythm function in an animal. Therefore, an assay employing a cell preparation, tissue or organ that maintains one or more indices of circadian rhythm function can be useful for assessing the effect of a PR2 antagonist or agonist on circadian rhythm function in an animal.

Such in vitro functional screening provides a relatively inexpensive alternative to animal screening that can be used to select potent candidate therapeutic molecules. In vitro functional screening for the ability of a PK2 receptor agonist or antagonist to modulate circadian rhythmicity can be performed using a variety of cell types that display an index of circadian rhythmicity and express PK2 receptor. Several cell types maintain circadian rhythm function outside of the organism from which they are isolated. For example, within the mammalian hypothalamus, the suprachiasmatic nucleus (SCN) contains a circadian clock for timing of diverse neuronal, endocrine and behavioral rhythms. By culturing cells from rat SCN on fixed microelectrode arrays, it has been shown that individual SCN neurons maintain spontaneous action potentials consistent with circadian rhythms for weeks (Welsh et al., Neuron 14:697-706 (1995). As shown herein, in Example V, PK2 receptor is highly expressed in mouse SCN. Therefore, cells from the SCN can be exemplary cells having an observable index of circadian rhythm function that express a PK2 receptor and can be used in the screening methods of the invention. The activity of isolated neurons, such as those from the SCN, can be measured using a variety of well-known methods, such as those described in Welsh, supra (1995). Cyclical change in neuron firing consistent with circadian rhythm is an example of an index of circadian rhythm that can be evaluated in the methods of the invention.

Another example of a cell that maintains circadian rhythm function outside of the organism from which it is isolated is a liver cell in which the circadian rhythm of the activity of metabolism enzymes is maintained. As described below, PK2 receptor is expressed in liver. Therefore, cells from the liver can be exemplary cells having an observable index of circadian rhythm function that express a PK2 receptor and can be used in the screening methods of the invention. The levels of enzyme activities, for example, the levels of metabolic enzymes and signal transduction enzymes can be measured using well-known methods. For example, the production or destruction of an enzyme substrate can be measured using a variety of analytical methods. Those skilled in the art will be able to select an appropriate method for measuring the activity of a particular enzyme. Cyclical change in the activity of cellular enzymes cellular consistent with circadian rhythm is an example of an index of circadian rhythm that can be evaluated in the methods of the invention.

Circadian rhythmicity also has been observed in cultured tissues explanted from peripheral organs (Yamazaki et al., Science 288:682-685 (2000)) and immortalized peripheral cells (Balsalobre et al., Cell 93:929-937 (1998) and Akashi et al., Genes Dev. 14:645-649 (2000)). As shown in Li et al, supra, 2001, PK2 receptor is expressed in several peripheral organs, including heart, testes, kidney, appendix, ovary, liver, and others. Those skilled in the art can obtain a variety of cell preparations, tissues or organs that express PK2 receptor, and determine whether circadian rhythmicity of a cell function, such as neuron firing, is maintained in a selected cell preparation, tissue or organ. Those skilled in the art will recognize that a variety of cell functions in addition to neuron firing can be maintained in a cell preparation, tissue or organ separated from an animal.

Gene transcription that is modulated by circadian rhythm is another example of an index of circadian rhythm that can be evaluated in the methods of the invention. The transcription of a variety of genes is regulated by circadian rhythm. Exemplary genes that are regulated by circadian rhythm include both genes whose products regulate the circadian clock (clock genes) and genes whose products mediate an animal's responses, such as physiological, endocrine, cellular and behavioral functions. Genes that are regulated by circadian rhythm vary among different animal species, although some clock genes are highly conserved among species. Examples of genes regulated by circadian rhythm include period-1, period-2, period-3, timeless, clock, cycle, double-time, cryptochrome-1, cryptochrome-2, PK2 and c-fos. For example, the c-fos gene has been shown to be regulated in the neural cells of rats after being exposed to a light source during the night to simulate an active day period (Kornhauser et al., Behav Genet., May, 26(3):221-40 (1996). Constant exposure to the light source resulted in a dramatically and rapidly increased amount of c-fos mRNA in the rat SCN. Those skilled in the art will be able to identify a gene that is regulated by circadian rhythm in an animal, and will be able to determine if such regulation is maintained in a cell preparation, tissue or organ. A variety of well-known methods can be used by those skilled in the art to monitor expression levels of genes during the circadian cycle in the presence or absence of a compound, such as a PK2 receptor agonist or antagonist.

A cellular index of circadian rhythm function can generally be determined by measuring a cellular activity specific for the cell type under study. For example, gene expression can be measured by well-known biochemical methods of detecting mRNA and polypeptide corresponding to a gene that is expressed in a circadian rhythm. Exemplary techniques include hybridization and PCR-based detection of mRNA and immunodetection of polypeptides, such as Western blotting and immunocytochemical methods.

A PK2 receptor antagonist or agonist, or PK2 promoter modulatory compound, also can be administered to an animal to determine if the compound modulates circadian rhythm. As used herein, the term “animal having an observable circadian rhythm function,” is intended to mean a human, veterinary animal or laboratory animal, including invertebrates, such as snails (for example, Bulla gouldiana); flies, such as the melon fly and fruit fly; reptiles, such as iguanas; fish, such as zebrafish; mammals, such as rodents, including, for example, rats, mice, hamsters and degus, rabbits, cats and non-human primates that exhibit or can be induced to exhibit, one or more indicia of circadian rhythm function. As described herein, for diurnal animals, a PK2 antagonist can beneficially promote sleep while a PK2 agonist can beneficially promote day-time associated behavior, such as alertness. Conversely, for nocturnal animals, a PK2 antagonist can beneficially promote night-time associated behavior, such as alertness, while a PK2 agonist can beneficially promote sleep or suppress REM sleep.

As used herein, the term “index of circadian rhythm function,” or in the plural, the term “indicia of circadian rhythm function,” is intended to mean an observable sign or indication of a physiological function, endocrine function, behavior or cellular function that is cyclical over the course of about 24 hours. Exemplary indicia of circadian rhythm function include cyclical changes in physiological functions, such as body temperature, autonomic regulation, metabolism, and sleep-wake cycles; cyclical changes in endocrine functions, such as secretion of hormones, including pineal melatonin secretion, ACTH-cortisol secretion, thyroid stimulating hormone secretion, and neuropeptide Y, serotonin secretion, and catecholamine secretion; and cyclical changes in behavior, such as movement (locomotor rhythm), mental alertness, memory, sensorimotor integration, and emotion, or cyclical changes in a cellular function, such as neuron firing or transcriptional control of gene expression.

Disorders of circadian rhythm function are characterized by alterations in physiological, endocrine and cellular functions and behavior. Indicia of abnormal circadian rhythm function include, for example, excessive day-time sleepiness, night-time arousals, and early awakening. In humans, clinically recognized circadian rhythm function disorders include Time-Zone Change (Jet Lag) Syndrome, Shift Work Sleep Disorder, Irregular Sleep-Wake Pattern, Delayed Sleep Phase Syndrome, Advanced Sleep Phase Syndrome, Non-24-Hour Sleep-Wake Disorder and the like.

As described in Example V, administration of PK2 during subjective night suppresses nocturnal behavior and produces daytime-associated behavior in rats. Accordingly, for nocturnal animals, a PK2 receptor agonist, or increase in PK2 gene expression, can be used to alter circadian rhythm by producing physiological, behavioral, or endocrine functions associated with subjective day-time, such as drowsiness, sleep or both. Accordingly, a PK2 receptor agonist, such as PK2, PK1 or a PK2 agonistic compound can be administered to a nocturnal animal in an amount sufficient to induce drowsiness, sleep or both, in the nocturnal animal. Conversely, a PK2 receptor antagonist, or decrease in PK2 gene expression, can be used to alter circadian rhythm in a nocturnal animal by producing physiological, behavioral, or endocrine functions associated with subjective night-time, such as increased alertness, wakefulness, activity, diligence, etc. For diurnal animals, a similar or an opposite treatment strategy can be used, such that by providing a PK2 receptor modulator or by modulating PK2 gene expression, a diurnal animal can be beneficially treated to alter circadian rhythm by producing physiological, behavioral, or endocrine functions associated with subjective day-time such as, for example, increased alertness, wakefulness or both. Thus, a PK2 receptor modulator can be used for treatment of conditions associated with increased or excessive sleep, such as narcolepsy, states of diminished vigilance and the like. In some embodiments of the invention, the modulator is a PK2 agonist, which, when administered to a diurnal subject produces physiological, behavioral or endocrine functions associated with subjective daytime, such as increased alertness, wakefulness or both. In additional embodiments of the invention, modulating PK2 gene expression means increasing the expression of PK2 in vivo such that there is produced in the diurnal animal physiological, behavioral or endocrine functions associated with subjective daytime, such as increased alertness, wakefulness or both. Conversely, a diurnal animal can be beneficially treated by modulating the level of PK2 receptor activity by providing a PK2 receptor modulator, or modulating PK2 gene expression, to alter circadian rhythm by producing physiological, behavioral or endocrine functions associated with subjective night-time, such as, for example drowsiness, sleep or both. Thus, an effective amount of a PK2 modulator can be used to treat conditions associated with reduced sleep, such as jet lag, shift worker syndrome and the like. In some embodiments, the PK2 modulator is a PK2 antagonist, which, when administered to the diurnal animal produces physiological, behavioral or endocrine functions associated with subjective night-time, such as drowsiness, sleep or both. In additional embodiments, modulating of PK2 gene expression entails reducing PK2 gene expression, in vivo such that there is produced in the diurnal animal physiological, behavioral or endocrine functions associated with subjective night-time, such as drowsiness, sleep or both.

Thus, the present invention provides methods for inducing or promoting physiological functions associated with subjective daytime in a diurnal mammalian subject. The method comprises administering to the diurnal mammalian subject an amount of a PK2 receptor modulator sufficient to induce in the subject at least one physiological function associated with subjective daytime in a diurnal mammalian subject. For diurnal subjects, such daytime-associated physiological functions include increased alertness, wakefulness, activity, vigilance, etc. Thus, methods according to the invention can be used to treat diurnal mammals experiencing various disorders, such as narcolepsy and other disorders discussed in more detail infra.

The present invention also provides methods for inducing or promoting physiological functions associated with subjective nighttime in a diurnal mammalian subject. The method comprises administering to the diurnal mammalian subject an amount of PK2 receptor modulator sufficient to induce in the subject at least one physiological function associated with subjective nighttime in a diurnal mammalian subject. For diurnal subjects, such nighttime-associated physiological functions include increased drowsiness, relaxation and sleep. Thus, the methods of the invention can be used to treat diurnal mammalian subjects suffering from a variety of disorders related to abnormal sleep patterns, such as insomnia and other disorders discussed in further detail infra.

Exemplary diurnal mammalian subjects that may be mentioned in connection with the methods of the invention include diurnal species of monkey, apes (including chimpanzees), humans, etc.

Thus, the present invention provides methods for inducing physiological functions associated with subjective daytime in a nocturnal mammalian subject. The method comprises administering to the nocturnal mammalian subject an amount of a PK2 receptor modulator sufficient to induce in the subject at least one physiological function associated with subjective daytime in a nocturnal mammalian subject. For nocturnal subjects, such daytime-associated physiological functions include increased drowsiness, relaxation and sleep.

The present invention also provides methods for inducing physiological functions associated with subjective nighttime in a nocturnal mammalian subject. The method comprises administering to the nocturnal mammalian subject an amount of PK2 receptor modulator sufficient to induce in the subject at least one physiological function associated with subjective nighttime in a nocturnal mammalian subject. For nocturnal subjects, such nighttime-associated physiological functions include increased alertness, wakefulness, activity, vigilance, etc.

Exemplary nocturnal mammalian subjects that may be mentioned include mice, rats, other rodents, cats, etc.

As described in Example XIV, administration of PK2 (a PK2 receptor agonist) to rats at night and during the daytime has a significant impact on REM sleep and a lesser impact on NREM sleep. A PK2 receptor agonist, such as PK2, may be administered to a mammal to modulate sleep-related parameters. The PK2 receptor agonist, such as PK2 may be administered at night or during the daytime. The effect of PK2 receptor agonist activity on the sleep patterns of nocturnal animals include suppression of REM sleep. Such suppression can be long-lasting, persisting for periods of up to 10 hours or more, whether the PK2 receptor agonist is administered during subjective day or night. Additionally, because REM sleep can be thought of as a period of central motor activation, suppression of REM sleep correlates with suppression of motor activity by PK2. It is to be expected that a PK2 receptor antagonist will have the opposite effect of a PK2 receptor agonist. It is likewise to be expected that a PK2 receptor agonist or antagonist will have modulatory effects on REM sleep and other sleep parameters. Thus the PK2 receptor is a drug target for sleep-wake disorder.

An animal can be evaluated for a variety of behaviors, as well as physiological and endocrine indices of circadian rhythm function. The method used for evaluating an animal will vary depending on the particular animal, for example, whether the animal is diurnal or nocturnal, and index evaluated. Behavioral indicia of circadian rhythm include a variety of activities, which can be activities that normally occur during the day or activities that normally occur during the night. Such activities vary considerably among animal species. For example, diurnal animals normally display sleep during the night and display motor activity during the day while nocturnal animals display sleep during the day and display motor activities during the night. A variety of daytime and nighttime activities of humans and other animals are well known and can be evaluated, for example, by viewing activity or behavior by eye, by imaging device, camera, video camera, tracking device, polygraph and the like, or by self-reporting of an activity or behavior, at one or more particular times. For example, described herein is the use of wheel-running as a behavioral index of circadian rhythm function of rats. Other types of spontaneous locomotor stereotypic activity, such as exploratory activity, can be assessed. Exemplary parameters that can be measured include time engaged in locomotor activity, time engaged in resting or sleeping, type of sleep (REM or NREM), distance traveled, number of footsteps and vertical counts (rearing).

For example, methods for evaluating an indicia of circadian rhythm function can involve determining the presence of increased or decreased sleepiness or alertness compared to normal. A “normal” amount of sleepiness or alertness is intended to mean an amount or quality of sleep observed or expected to be observed in an animal free from any circadian rhythm disorder. An animal having normal circadian rhythm can have, for example, an expected number of hours of sleep per 24 hour period, an expected amount of REM sleep, NREM sleep or both, an expected number of waking episodes, an expected number of daytime naps, and the like, that will be dependent on the animal species and characteristics of an individual, such as age, physical condition, prior injury or disease, medication and the like.

Physiological indicia of circadian rhythm function can generally be determined by physical examination of the individual, for example using an instrument or measuring device, such as measuring body temperature using a thermometer at a particular time of day. Other examples include using electrodes to measure brain waves to assess sleep at a particular time of day and using a movement or heart rate monitor to assess physical activity of animals at a particular time of day. The time of day selected for measurement can depend on the particular animal species. For example, diurnal and nocturnal animals will have cyclic changes in physiological indicia of circadian rhythm function that correspond with their normal waking and sleeping cycles.

Endocrine indicia of circadian rhythm function can generally be determined using a biochemical test, such as measuring the amount of a molecule, such as a hormone, contained in an individual's bodily fluid at a particular time of day. For example, the adrenal glands produce molecules, such as cortisol, in a circadian rhythm. Normally, cortisol output is highest in the morning, approximately mid-value throughout the day, and then drops to a nearly negligible level during the night. A variety of other molecules are known to be produced in a circadian rhythm, with peak and through levels occurring at particular times within the 24-hour day depending on the particular molecule. As an indicia of circadian rhythm function, the level of a molecule that is produced in the body in a circadian rhythm can be determined and compared to a known standard level of that molecule expected to be present in a bodily fluid at a particular time of day. In addition, the level of such a molecule can be measured at intervals or continuously over a period of time sufficient to assess whether the level of the molecule is modulated in a circadian rhythm. Exemplary bodily fluids that can be collected and tested for a level of a particular molecule include blood, urine and saliva.

An index of circadian rhythm function also can be observed by viewing the output of a variety of attachable or implantable devices designed to measure physiological, behavioral, or endocrine functions. An exemplary implantable device is an electrode useful for measuring neurological activity in laboratory animals (for example, to determine the light response of the SCN). An exemplary attachable device is a surface electromyography instrument useful for measuring electrical activity of individual muscles or muscle groups (for example, to determine the sleep or wake state of an individual). Signals from such devices can be amplified, and analyzed by a computer. Indicia of circadian rhythm can be determined qualitatively or quantitatively and animals can generally be tested prior to and after treatment with a PK2 receptor agonist or antagonist, compound that modulates PK2 promoter activity or carrier.

In evaluating whether a PK2 receptor antagonist or agonist, or PK2 promoter activity modulator modulates circadian rhythm, a difference in a value of an index of circadian rhythm function before and after treatment with a compound can be determined. The amount of a PK2 receptor antagonist or agonist, or PK2 promoter activity modulator effective to modulate circadian rhythm is an amount effective to modulate the determined index of circadian rhythm function by at least 10%. For example, the determined index of circadian rhythm function can be increased or reduced by at least 20%, at least 50%, such as at least 80%, in at least some treated animals.

Methods for evaluating circadian rhythm in an animal are useful for both diagnosing a variety of circadian rhythm disorders to determine if an individual is a candidate for treatment with a PK2 receptor antagonist or agonist, as well as to evaluate an individual's response to administration of a PK2 receptor antagonist or agonist or PK2 promoter modulatory compound.

Methods for diagnosing circadian rhythm disorders are well known to those skilled in the art. Circadian rhythm disorders are described, for example, in The Merck Manual of Diagnosis and Therapy, 17^{th} edition, Beers and Berkow (Eds.), Merck & Co. (1999). Circadian rhythm disorders that can be treated by a PK2 receptor antagonist or agonist, or PK2 promoter modulatory compound, include primary (idiopathic) or secondary (symptomatic, related to known disorders) or drug-induced circadian rhythm disorders, which can be hereditary or sporadic. In particular, circadian rhythm sleep disorders are disorders related to the timing of sleep within the 24-hour day. Some of these disorders are influenced by the timing of the sleep period that is under the individual's control (for example, shift work or time zone change). Others are disorders of neurological mechanisms (for example, irregular sleep-wake pattern and advanced sleep phase syndrome). Those skilled in the art will be able to identify individuals having circadian rhythm function disorders that could benefit from treatment with a PK2 receptor antagonist or agonist, including those having circadian rhythm sleep disorders that could benefit from treatment with either a PK2 receptor antagonist that promotes alertness, or a PK2 receptor agonist that promotes sleep.

As described above, a variety of circadian rhythm function disorders can lead to reduced quality or quantity of sleep in an individual. Human circadian rhythm sleep disorders, such as non-24-hour sleep-wake syndrome, rapid time-zone change syndrome, work-shift syndrome, delayed phase sleep syndrome, advanced sleep phase syndrome, irregular sleep-wake pattern syndrome and syndrome associated with decreased amplitude, are characterized by abnormal sleeping and waking patterns, and can lead to the need for either increased alertness or increased sleep. In addition, medical and psychiatric conditions, such as chronic pain and depression, can affect circadian rhythm and cause reduced quality or quantity of sleep. In addition, because PK2 expression in the SCN can encode day length information, as shown in Example XI, modulation of PK2 receptor activity can be useful for altering seasonal rhythm regulation to treat disorders such as seasonal affective disorder. Further, medications used for treating a variety of conditions can affect circadian rhythm and cause reduced quality or quantity of sleep. A PK2 receptor antagonist or agonist, or PK2 promoter modulatory compound, identified by the methods of the invention as a compound that modulate circadian rhythm can be used to treat such disorders in animals, in particular humans.

A PK2 receptor agonist or PK2 promoter modulatory compound that increases PK2 gene expression can be used to modulate the circadian rhythm of an animal such that the animal experiences increased alertness or reduced sleep. An animal or individual to be treated with a PK2 receptor agonist, including PK2, can be one that would benefit physically or psychologically from increased alertness. For example, increased alertness may be desired in an individual having sleepiness, a tendency to fall asleep, or having a sense of excessively deep sleep. A need for alertness can arise in an individual having a life-style induced circadian rhythm disorder, such as a condition caused by irregular work hours or shift work. An individual may desire increased alertness to enhance performance in mental or physical activities, such as long distance driving, shift work and study. A need for alertness can also be caused by a physiologically based circadian rhythm disorder that causes excessive daytime sleepiness, as well as adverse drug reactions. The compositions identified using the methods of the invention can thus be used to ameliorate the symptoms of such disorders and conditions.

A PK2 receptor antagonist or PK2 promoter modulatory compound that decreases PK2 gene expression can be used to modulate the circadian rhythm of an animal such that the animal experiences increased sleep or reduced alertness. Therefore, an animal or individual to be treated with a PK2 receptor antagonist can be one that could benefit physically or psychologically from increased quality or quantity of sleep. For example, a animal or individual having reduced or insufficient quality or quantity of sleep, such as reduced ability to fall asleep or stay asleep, having the tendency to awaken earlier than desired in the morning or having a sense of light or unrefreshing sleep could benefit physically or psychologically from increased quality or quantity of sleep. The compositions identified using the methods of the invention can thus be used to ameliorate the symptoms of such disorders and conditions.

Those skilled in the art can determine other conditions for which it is appropriate to administer a PR2 receptor antagonist or antagonist identified by the methods of the invention to modulate circadian rhythm, and can monitor the safety and efficacy of the therapy.

Compounds such as PK2 receptor antagonists and agonists and PK2 promoter modulatory compounds can be evaluated for their ability to modulate circadian rhythm by administering to an animal. Such compounds can be potential therapeutic compounds that can be administered to individuals, such as those with conditions associated with abnormal circadian rhythm. Therefore, the methods of the invention can involve administering a pharmaceutical composition containing a PR2 receptor agonist or antagonist or other compound to an animal, including a veterinary animal, research animal or human. For determining if a compound modulates an index of circadian rhythm, the methods of the invention can be practiced using a variety of animals that have a detectable index of circadian rhythm function. Exemplary animals and indices of circadian rhythm are described above. For preclinical studies, the methods of the invention can be practiced with animals that serve as credible models of human disease, such as non-human primates, pigs, dogs, cats, hamsters and rodents (for example rats, mice and guinea pigs). Those skilled in the art understand which animals serve as appropriate models for a human disease of interest.

The identified compounds can be formulated and administered in a manner and in an amount appropriate for the condition to be treated; the weight, gender, age and health of the individual; the biochemical nature, bioactivity, bioavailability and side effects of the particular compound; and in a manner compatible with concurrent treatment regimens. An appropriate amount and formulation for a particular therapeutic application in humans can be extrapolated based on the activity of the compound in the in vitro binding and signaling assays described herein, or from recognized animal models of the particular disorder.

The total amount of a compound, including a therapeutic compound, can be administered as a single dose or by infusion over a relatively short period of time, or can be administered in multiple doses administered over a more prolonged period of time. Additionally, the compound can be administered in a slow-release matrix, which can be implanted for systemic delivery at or near the site of the target tissue. Contemplated matrices useful for controlled release of compounds, including therapeutic compounds, are well known in the art, and include materials such as DepoFoam™, biopolymers, micropumps, and the like.

Compounds, including therapeutic compounds, can be administered to a mammal by routes known in the art including, for example, intravenously, intramuscularly, subcutaneously, intraorbitally, intracapsularly, intraperitoneally, intracistemally, intra-articularly, intracerebrally, orally, intravaginally, rectally, topically, intranasally, or transdermally. Preferred routes for human administration are oral and intravenous administration, with oral routes particularly preferred.

Generally, compounds, including therapeutic compounds, are administered to an animal as a pharmaceutical composition comprising the compound and a pharmaceutically acceptable carrier. The choice of pharmaceutically acceptable carrier depends on the route of administration of the compound and on its particular physical and chemical characteristics. Pharmaceutically acceptable carriers are well known in the art and include sterile aqueous solvents such as physiologically buffered saline, and other solvents or vehicles such as glycols, glycerol, oils such as olive oil and injectable organic esters. A pharmaceutically acceptable carrier can further contain physiologically acceptable compounds that stabilize the compound, increase its solubility, or increase its absorption. Such physiologically acceptable compounds include carbohydrates such as glucose, sucrose or detrains; antioxidants, such as ascorbic acid or glutathione; chelating agents; and low molecular weight proteins.

For applications that require the compounds and compositions to cross the blood-brain barrier, or to cross cell membranes, formulations that increase the lipophilicity of the compound are particularly desirable. For example, the compounds of the invention can be incorporated into liposomes (Gregoriadis, Liposome Technology, Vols. I to III, 2nd ed. (CRC Press, Boca Raton Fla. (1993)). Liposomes, which consist of phospholipids or other lipids, are nontoxic, physiologically acceptable and metabolizable carriers that are relatively simple to make and administer.

For treating a circadian rhythm function disorder treatment, more than one therapeutic approach or compound can be provided to an individual for maximal symptom control. Thus, for use in modulating circadian rhythm function, a PK2 receptor antagonist or agonist or PK2 promoter modulator can advantageously be administered concurrently or sequentially with another therapeutic mode or formulated with a second compound that controls the same or related symptoms. For example, in treating circadian rhythm sleep disorders, a PK2 receptor antagonist or agonist can be administered while an individual is receiving light therapy, drug therapy and the like. Contemplated methods of modulating circadian rhythm include administering PK2 receptor antagonists or agonists alone, in combination with, or in sequence with, such other compounds. The skilled clinician will be able to determine concurrent or sequential therapies appropriate for use with a PK2 receptor antagonist or agonist.

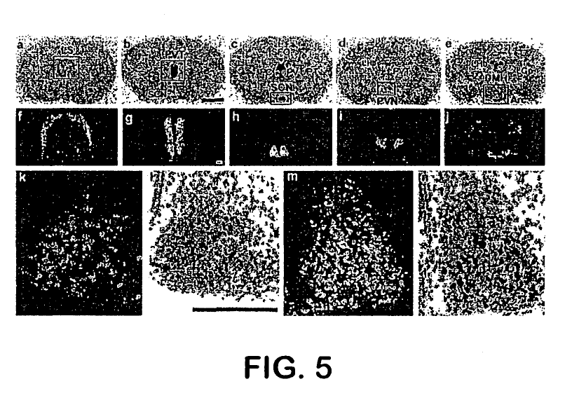

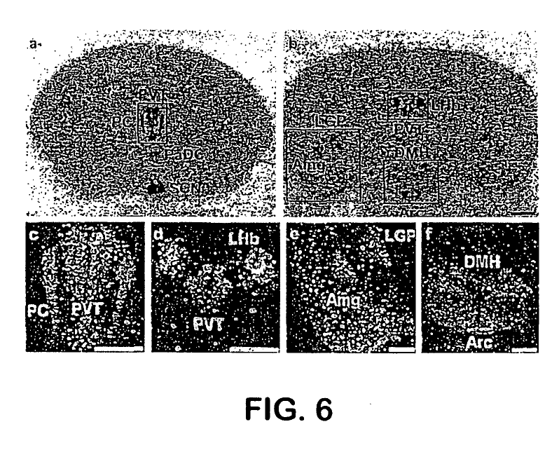

It is expected that the PK2 receptor antagonists and agonists identified using the screening methods of the invention will have beneficial activities apart from, or in addition to, modulating circadian rhythm function. As described in Example V, high levels of PK2 receptor expression have been observed in discrete locations in the brain. In particular, PK2 receptor is expressed at high levels in SCN, paraventricular nucleus of hypothalamus (PVN), dorsal medial nucleus of hypothalamus (DMH), paraventricular and paratenial nuclei of thalamus (PVT/PT), paracentral thalamic nucleus (PC), lateral habenular nucleus (LHb) and lateral septal nucleus (LS) (see FIGS. 5 and 6).

The invention also provides an isolated nucleic acid molecule encoding a mouse PK2 receptor polypeptide. As described above, a mouse PK2 receptor, such as that having the amino acid sequence referenced as SEQ ID NO:2, which is encoded by the nucleotide sequence referenced as SEQ ID NO:1, is able to bind to PK2 and signal through a G-protein coupled signal transduction pathway and modulate circadian rhythm in mice.

The invention further provides an isolated nucleic acid molecule containing a PK2 gene promoter, which can be operatively linked to a heterologous nucleotide sequence. Exemplary human and mouse PK2 gene promoters include the human PK2 promoter (SEQ ID NO:14), human PK2 2.8 kb promoter (SEQ ID NO:15), mouse PK2 promoter (SEQ ID NO:16), mouse 2.8 kb PK2 promoter (SEQ ID NO:17), mouse 188 bp PK2 promoter (SEQ ID NO:18) and a 72 bp promoter containing the 4 human E-box enhancer elements of the human PK2 promoter linked together (SEQ ID NO:19).

As shown herein, PK2 gene expression is regulated in a circadian fashion (see Examples I, II and IV). As such, the PK2 promoter can be useful for cyclically regulating a variety of genes in a circadian fashion. For example, a PK2 promoter can be used to modulate expression of a therapeutic gene. Cyclic expression of a therapeutic gene can be advantageous, for example, when the presence of a gene product is most beneficial during a particular time of day, such as daytime when the individual is active.

A PK2 promoter can be operatively linked to a variety of heterologous nucleotide sequences, which can be, for example, a reporter nucleic acid, antisense nucleic acid, nucleic acid encoding a therapeutic polypeptide, or other sequence to be regulated in a circadian fashion. Exemplary reporter nucleic acids are described above, in relation to use of a PK2 promoter in screening methods.

The nucleic acid molecules of the invention also are suitable for a variety of screening applications. For example, the invention nucleic acid molecules can be used in the screening assays described herein. For example, an invention nucleic acid molecule containing a PK2 gene promoter can be used to identify compounds that modulate PK2 gene promoter activity. In addition, an invention nucleic acid molecule encoding mouse PK2 receptor can be expressed in a cell, which can be used in a screening assay, or the encoded PK2 polypeptide can be isolated for use in a screening assay. An invention nucleic acid molecule encoding mouse PK2 receptor also can be used as a probe or primer to identify and isolate PK2-encoding nucleic acid molecules from other species, or to identify structurally related molecules.

As used herein, the term “isolated nucleic acid molecule” is intended to mean that the nucleic acid molecule is altered, by the hand of man, from how it is found in its natural environment. For example, an isolated nucleic acid molecule can be a molecule operatively linked to an exogenous nucleic acid sequence. An isolated nucleic acid molecule can also be a molecule removed from some or all of its normal flanking nucleic acid sequences.

An isolated molecule can alternatively, or additionally, be a “substantially pure” molecule, in that the molecule is at least 60%, 70%, 80%, 90 or 95% free from cellular components with which it is naturally associated. An isolated nucleic acid molecule can be in any form, such as in a buffered solution, a suspension, a lyophilized powder, attached to a solid support (for example, as a component of a DNA array), or in a cell.

As used herein, the term “nucleic acid molecule” refers to a polynucleotide, including an oligonucleotide, of natural or synthetic origin, which can be single- or double- can correspond to genomic DNA, cDNA or RNA, and can represent either the sense or antisense strand or both.

The term “nucleic acid molecule” is intended to include nucleic acid molecules that contain one or more non-natural nucleotides, such as nucleotides having modifications to the base, the sugar, or the phosphate portion, or having one or more non-natural linkages, such as phosphorothioate linkages. Such modifications can be advantageous in increasing the stability of the nucleic acid molecule, particularly when used in hybridization applications.

Furthermore, the term “nucleic acid molecule” is intended to include nucleic acid molecules modified to contain a detectable moiety, such as a radiolabel, a fluorochrome, a ferromagnetic substance, a luminescent tag or a detectable binding agent such as biotin. Nucleic acid molecules containing such moieties are useful as probes for detecting the presence or expression of PK2 receptor nucleic acid molecule.

A nucleic acid molecule of SEQ ID NO:1 does not consist of the exact sequence of an EST present in publicly available databases, including the sequences designated by GenBank Accession numbers XM_{—}066104 and AL121755. Similarly, a nucleic acid molecule of SEQ ID NO:14 does not consist of the exact sequence of an EST present in publicly available databases, including the sequences designated by GenBank Accession numbers AC096970.2, AF182067.1 and AC010207.18.

The invention further provides isolated oligonucleotides that contain at least 17 contiguous nucleotides from SEQ ID NOS:1 and 14, or the complement thereof. The oligonucleotides of the invention are thus of sufficient length to be useful as sequencing primers, PCR primers and hybridization probes to detect or isolate nucleic acid molecules. For example, an oligonucleotide of SEQ ID NO:1 can be used to detect or isolate nucleic acid molecules encoding PK2 receptor polypeptides, and are also useful as therapeutic antisense reagents to inhibit PK2 receptor expression. Such an oligonucleotide can, but need not, encode PK2 receptor polypeptides that are able to modulate circadian rhythm function in an animal. An oligonucleotide of SEQ ID NO:14 can be used to detect or isolate nucleic acid molecules containing a PK2 promoter and are also useful in the screening methods of the invention for identifying a compound that modulates circadian rhythm. Those skilled in the art can determine the appropriate length and sequence of a oligonucleotide of the invention for a particular application.

As used herein, the term “oligonucleotide” refers to a nucleic acid molecule that contains at least 17 contiguous nucleotides from the reference sequence and which can, but need not, encode a functional polypeptide. Thus, a oligonucleotide of the invention can contain at least 17, 20, 22 or 25 contiguous nucleotides, such as at least, or not more than, 30, 40, 50, 60, 70, 80, 90, 100, 125, 150, 175, 200, 250, or 300 contiguous nucleotides from SEQ ID NOS:1 or 14, or from their complement. An oligonucleotide of SEQ ID NO:1 does not consist of the exact sequence of an EST present in publicly available databases, including the sequences designated by GenBank Accession numbers XM_{—}066104 and AL121755. Similarly, an oligonucleotide of SEQ ID NO:14 does not consist of the exact sequence of an EST present in publicly available databases, including the sequences designated by GenBank Accession numbers AC096970.2, AF182067.1 and AC010207.18.

For certain applications, it is desirable to use isolated oligonucleotide molecules of the invention that specifically hybridize to a target nucleic acid molecule. For example, for detecting PK2 receptor expression in a sample, it is desirable to use isolated oligonucleotide molecules of the invention that specifically hybridize to a nucleic acid molecule encoding a PK2 receptor. Similarly, for detecting a non-expressed nucleic acid, such as a PK2 promoter, in a sample, it is desirable to used isolated oligonucleotides molecules of the invention that specifically hybridize to a nucleic acid molecule containing a PK2 promoter.

As used herein, the term “specifically hybridize” refers to the ability of a nucleic acid molecule to hybridize, under moderately stringent conditions as described above, to the reference PK2 receptor nucleic acid molecule (SEQ ID NO:1) or PK2 promoter nucleic acid molecule (SEQ ID NO:14), without hybridization under the same conditions with nucleic acid molecules that are not PK2 receptor or PK2 promoter nucleic acid molecules, respectively, such as actin cDNA.

As used herein, the term “stringent conditions” refers to conditions equivalent to hybridization of a filter-bound nucleic acid molecule to a nucleic acid in a solution containing 50% formamide, 5× Denhart's solution, 5×SSC, 0.2% SDS at 42° C., followed by washing the filter in 0.1×SSC and 0.1% SDS at 65° C. twice for 30 minutes. Equivalent conditions to the stringent conditions set forth above are well known in the art, and are described, for example in Sambrook et al., Molecular Cloning: A Laboratory Manual, Cold Spring Harbor Laboratory, New York (1992).

Methods for preparing an invention nucleic acid molecule, or a nucleic acid molecule that is a PK2 receptor agonist or antagonist include well known chemical synthesis methods, such as automated methods, and recombinant methods, such as those described in Sambrook et al., Molecular Cloning: A Laboratory Manual, 3rd ed., Cold Spring Harbor Press, Plainview, N.Y. (2001); Ausubel et al. (Current Protocols in Molecular Biology (Supplement 47), John Wiley & Sons, New York (1999)). An exemplary method for preparing an isolated mouse PK2 receptor nucleic acid, human PK promoter region nucleic acid, or other nucleic acid molecules involves amplification of the nucleic acid molecule using specific primers and the polymerase chain reaction (PCR). Using PCR, a nucleic acid molecule having any desired boundaries can be amplified exponentially starting from only a few DNA or RNA molecules, such as from a single cell. PCR methods, including methods of isolating homologs of a given nucleic acid molecule in other species using degenerate primers, are well known in the art.

Alternatively, an isolated nucleic acid molecule can be prepared by screening a library, such as a genomic library, cDNA library or expression library, with a detectable nucleic acid molecule or with an antibody. Human libraries, and libraries from a large variety of mammalian species, are commercially available or can be produced from species or cells of interest. The library clones identified as containing a particular nucleic acid molecule can be isolated, subcloned or sequenced by routine methods.

Furthermore, an isolated invention nucleic acid molecule, or nucleic acid molecule used in the methods of the invention, can be prepared by direct synthetic methods. For example, a single stranded nucleic acid molecule can be chemically synthesized in one piece, or in several pieces, by automated synthesis methods known in the art. The complementary strand can likewise be synthesized in one or more pieces, and a double-stranded molecule made by annealing the complementary strands. Direct synthesis is particularly advantageous for producing relatively short molecules, such as probes and primers, and also for producing nucleic acid molecules containing modified nucleotides or linkages.

The invention provides a vector containing an isolated nucleic acid molecule encoding a mouse PK2 receptor polypeptide (SEQ ID NO:1). The invention also provides a vector containing an isolated nucleic acid molecule containing a human PK2 gene promoter region (SEQ ID NO:14). Exemplary vectors include vectors derived from a virus, such as a bacteriophage, a baculovirus or a retrovirus, and vectors derived from bacteria or a combination of bacterial sequences and sequences from other organisms, such as a cosmid or a plasmid. The vectors of the invention will generally contain elements such as an origin of replication compatible with the intended host cells; transcription termination and RNA processing signals; one or more selectable markers compatible with the intended host cells; and one or more multiple cloning sites. Optionally, the vector will further contain sequences encoding tag sequences, such as GST tags, and/or a protease cleavage site, such as a Factor Xa site, which facilitate expression and purification of the encoded polypeptide.

The choice of particular elements to include in a vector will depend on factors such as the intended host cells; the insert size; whether expression of the inserted sequence is desired; the desired copy number of the vector; the desired selection system, and the like. The factors involved in ensuring compatibility between a host cell and a vector for different applications are well known in the art.

In applications in which the vectors are to be used for recombinant expression of the encoded polypeptide, the isolated nucleic acid molecules will generally be operatively linked to a promoter of gene expression which may be present in the vector or in the inserted nucleic acid molecule. The choice of promoter to operatively link to an invention nucleic acid molecule will depend on the intended application, and can be determined by those skilled in the art. For example, if a particular gene product may be detrimental to a particular host cell, it may be desirable to link the invention nucleic acid molecule to a regulated promoter, such that gene expression can be turned on or off. Alternatively, it may be preferred to have expression driven by either a weak or strong constitutive promoter. Exemplary promoters suitable for mammalian cell systems include, for example, the SV40 early promoter, the cytomegalovirus (CMV) promoter, the mouse mammary tumor virus (MMTV) steroid-inducible promoter, and the Moloney murine leukemia virus (MMLV) promoter. Exemplary promoters suitable for bacterial cell systems include, for example, T7, T3, SP6, lac and trp promoters.

Also provided are cells containing an isolated nucleic acid molecule encoding a mouse PK2 receptor polypeptide. The isolated nucleic acid molecule will generally be contained within a vector. The isolated nucleic acid molecule can be maintained episomally, or incorporated into the host cell genome.

The cells of the invention can be used, for example, for molecular biology applications such as expansion, subcloning or modification of the isolated nucleic acid molecule. For such applications, bacterial cells, such as laboratory strains of E. coli, are useful, and expression of the encoded polypeptide is not required.

The cells of the invention can also advantageously be used to recombinantly express and isolate the encoded polypeptide. For such applications bacterial cells (for example, E. coli), insect cells (for example, Drosophila and Spodoptera fugiperda), yeast cells (for example, S. cerevisiae, S. pombe, or Pichia pastoris), and vertebrate cells (for example, mammalian primary cells and established cell lines, such as CHO, 293 and COS cells; and amphibian cells, such as Xenopus embryos and oocytes).

The invention provides an isolated mouse PK2 receptor polypeptide, which is encoded by the nucleotide sequence referenced as SEQ ID NO:1 and degenerate variants thereof. The isolated mouse PK2 receptor polypeptide can be used in assays to identify PK2 receptor antagonists and agonists.

The invention also provides compositions suitable for use in assays to identify PK2 receptor agonists and antagonists. Suitable compositions contain an isolated tissue, isolated cell or cell preparation, containing a mouse PK2 or PK1 receptor and a PK2 or PK1 polypeptide. A composition suitable for screening can contain either a PK2 or PK1 receptor in combination with either a PK2 or PK1 polypeptide. For example, suitable combinations include a PK2 receptor together with PK1 or PK2, as well as a PK1 receptor together with PK1 or PK2. Any of these combinations can be used to identify a compound that is a PK2 receptor antagonist or agonist because PK1 receptor and PK2 receptor are structurally similar and both bind to PK1 and PK, as well as to PK1/PK2 chimeras, such as SEQ ID NOS:20 and 21. A PK2 receptor contained in a composition of the invention does not include the exact sequence of an EST present in publicly available databases, including the amino acid sequences designated by GenBank Accession numbers XM_{—}066104 and AL121755. Similarly, a PK1 receptor contained in a composition of the invention does not include the exact sequence of an EST present in publicly available databases, including the amino acid sequences designated by GenBank Accession number NM_{—}021381, AF236082 and XM_{—}066104.

A PK2 or PK1 receptor contained in the compositions can be, for example, a mammalian PK2 or PK1 receptor such as a mouse or human receptor. Exemplary mouse PK2 and PK1 receptor polypeptide sequences include those referenced as SEQ ID NOS:2 and 4, respectively. A PK2 or PK1 polypeptide contained in the compositions binds to a PK2 receptor, a PK1 receptor, or both. Exemplary PK2 polypeptides include human PK2s referenced as SEQ ID NOS:5 and 6 and mouse PK2s referenced as SEQ ID NOS:7 and 8. Exemplary PK1 polypeptides include human PK1 referenced as SEQ ID NO:9 and mouse PK1 referenced as SEQ ID NO:10. Orthologs of PK1 and PK2, such as Bv8 (SEQ ID NOS:11 and 12) and MIT1 (SEQ ID NO:13) that bind to a PK2 receptor or PK1 receptor also can be contained in a composition of the invention. A PK1 or PK2 in a composition of the invention optionally can be detectably labeled.

The compositions of the invention can also include crude or partially purified lysates or extracts of cells containing PK2 receptor or PK1 receptor, and reconstituted signaling systems containing PK2 receptor or PK1 receptor. Artificial signaling systems include, for example, natural or artificial lipid bilayers, such as a liposome or micelle, which promote an active conformation of PK2 receptor. The compositions can further contain cellular fractions or isolated components necessary for producing and detecting a desired predetermined signal.

A composition of the invention further can contain both PK1 receptor and PK2 receptor and either or both PK1 and PK2, or another PK1 receptor or PK2 receptor agonist, such as a PK2/PK1 chimera.

It is understood that modifications which do not substantially affect the activity of the various embodiments of this invention are also included within the definition of the invention provided herein. Accordingly, the following examples are intended to illustrate but not limit the present invention.

### EXAMPLE I

Rhythmic Expression of Prokineticin 2 (PK2) in the Suprachiasmatic Nucleus (SCN)

This example shows that PK2 mRNA is expressed rhythmically in the SCN of mouse.

In situ hybridization was used to detect a mouse PK2 mRNA transcript in the mouse brain. Antisense and sense riboprobes containing the coding region of mouse PK2 or the 3′UTR (untranslated region) of mouse PKR2 were generated. The 3′UTR of the PKR1 and PKR2 were used as these receptors are over 80% identical at nucleic acid sequence level in their coding regions. In situ hybridizations were processed as described in (Winzer-Serhan et al., Brain Res. Protocols 3:229-41 (1999)). The mRNA distributions were analyzed in autoradiograms and emulsion-dipped sections. Specific hybridization signals were quantitatively analyzed using a video-based computer image analysis system (MCID, Imaging Research, St. Catharine's, Ontario, Canada). A calibration curve of optical density versus radioactivity (dpm/mg tissue wet weight) was constructed using ¹⁴C-standards. Specific hybridization signals in SCN were obtained by subtracting background values obtained from adjacent brain areas that have no hybridization signal. Data were normalized with respect to the differences between signal intensities in equal areas of SCN.

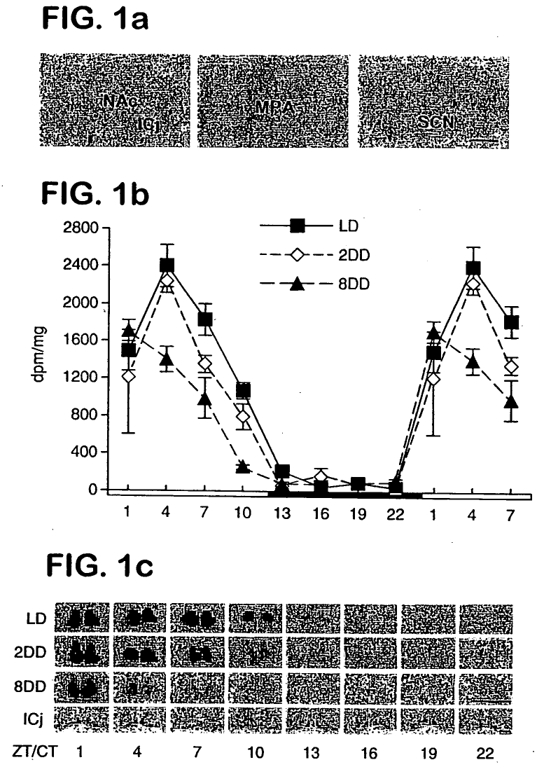

The results from these in situ hybridization studies indicated that PK2 mRNA is present in the SCN and other discrete brain areas including the islands of Calleja, medial preoptic area of the hypothalamus, and the shell of the nucleus accumbens. FIG. 1a shows PK2 mRNA expression in coronal sections (20 μm) of the mouse brain. The nucleus accumbens (NAc); islands of Calleja (ICj); medial preoptic area (MPA) and suprachiasmatic nucleus (SCN) are shown in FIG. 1a (Scale bar=2 mm).

To determine the time dependence of the presence of PK2 mRNA in the SCN, male adult C57B16 mice (Taconic Farms, N.Y.) were entrained under a 12-hr light:12-hr dark (LD) cycle. Quantification of PK2 mRNA in the SCN at various time points indicated the presence of circadian oscillation profiles in LD. Animals were studied under LD, or constant darkness for 2 days (2DD) or 8 days (8DD). FIG. 1b shows temporal profiles of PK2 mRNA in SCN; each value is the mean±SEM of 3-6 animals, with data at ZT/CT1-7 being double plotted. Shaded and closed horizontal bars indicate light and dark periods, respectively. As indicated by FIG. 1b, it was observed that PK2 mRNA is highest during light phase (ZT1-ZT7) (ZT, Zeitgeber time; ZTO=light on, ZT12=light off), and lowest during dark phase (ZT13-ZT22). The oscillation magnitude of PK2 mRNA was observed to be high, with the peak level at least 50-fold higher than the lowest level. FIG. 1c shows representative images of PK2 mRNA expression described in FIG. 1b (Scale bar=1 mm). FIG. 1c shows that PK2 mRNA level was essentially undetectable in SCN during dark phase. Oscillation of PK2 expression was not detected in other PK2 mRNA-positive areas such as the medial preoptic area or the islands of Calleja (FIG. 1c). The closely-related PK1 mRNA was not expressed at a detectable level in the brain areas examined, including the SCN.

The oscillation of PK2 mRNA was maintained under constant darkness (DD) (FIG. 1b, c). After 2 days in DD, PK2 mRNA levels were high from CT1-CT7 (CT, circadian time; CT0, subjective light on; CT12, subjective light off), and remained low from CT13-CT22. However, after 8 days in DD, the period of low PK2 expression expanded from about 9-hr in LD to 12-hr, with a slight reduction in peak level. The robust circadian profile of PK2 mRNA in the SCN indicates that PK2 regulates SCN circadian pacemaker and/or its output.

In summary, this example shows that PK2 mRNA presence in the SCN of mouse corresponds to a circadian oscillatory profile.

### EXAMPLE II

Regulation of PK2 Transcription by Clock Genes

This example shows that PK2 gene transcription is regulated by clock genes.

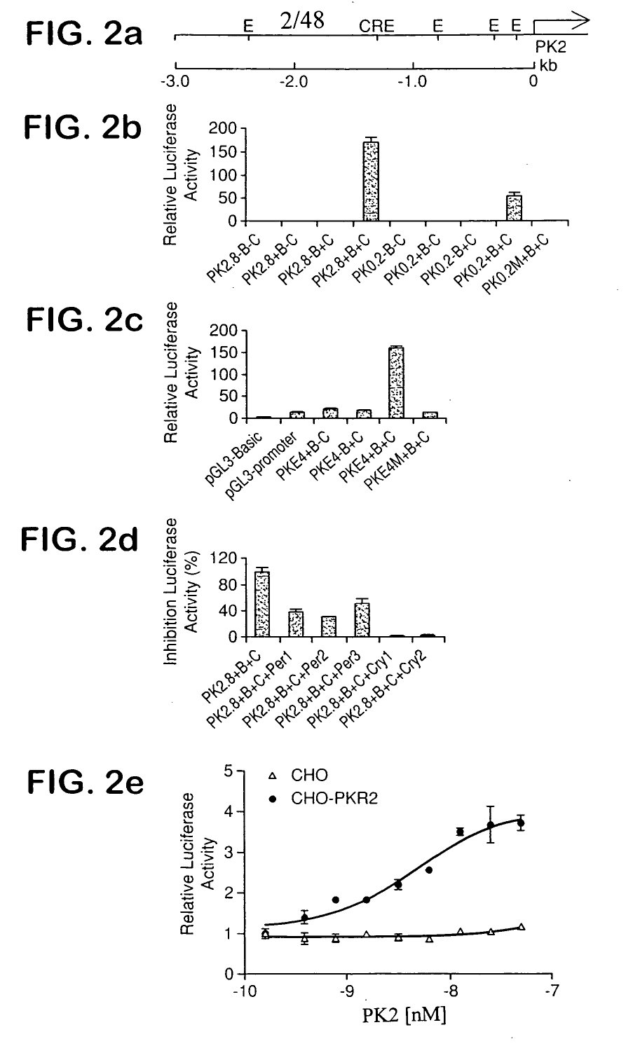

The promoter sequence of human PK2 gene was examined (Jilek et al., Gene 256:189-95 (2000)) to determine the role of E-box enhancers in CLOCK-BMAL1-mediated transcriptional activation (Gekakis et al., Science 280:1564-69 (1998); and Hogenesch et al., Proc. Natl. Acad. Sci. USA 95;5474-79 (1998)). FIG. 2a shows the location of four E-boxes (E) and cAMP-responsive element (CRE) identified within 2.4 kb of the 5′-flanking region of the mouse PK2 gene. The numbered axis represents distance in kilo base pairs (kb) from the putative transcription start site, marked as 0. All four E-boxes are conserved in the 5′-flanking region of mouse PK2 gene. A GenBank search indicated that these four E-box elements are conserved in the 5′-flanking sequence of the mouse PK2 gene, including the approximate location of each of the four E-boxes. No E-box sequence was identified in 5 kb 5′-flanking region of the closely-related PK1 gene.

To examine the ability of CLOCK:BMAL1 heterodimers to drive PK2 transcription through E-box enhancers, a 2.8 kb fragment of the mouse PK2 5′-flanking region was cloned into a promoterless luciferase reporter vector (pGL3-Basic).

The ability of transcription factors to promote transcriptional activity of the mouse PK2 promoter was examined. Transcriptional activation of luciferase reporter linked to 2.8 kb (PK2.8-Luc) and 200 bp (PK0.2-Luc) of the 5′-flanking region of mouse PK2 gene was studied. In addition, a luciferase reporter was linked to a mutated E-box (GGATCT)(PK0.2M-Luc).

HEK293 and NIH3T3 cells were grown in Dulbecco's Modified Eagle Medium (DMEM) supplemented with 10% fetal bovine serum. Cells were plated and transfected with Lipofectamine (Invitrogen, Carlsbad, Calif.) in six-well plates. 10 ng of firefly luciferase reporter plasmid, 250 ng of mBmal1 and mClock and 500 ng of mPer1, mPer2, mPer3, mCry1, and mCry2 were used. The total amount of DNA (1 mg per well) was adjusted with pcDNA3.1 vector as carrier. 48-hr after transfection, cells were washed and lysed with 200 μl reporter lysis buffer. Portion of the cell extract (20 μl) was mixed with Luciferase Assay Reagent (Promega) and the reaction monitored for 10 s in a Monolight 2010 luminometer (Analytical Luminescence Laboratory, San Diego, Calif.). For Chinese Hamster Ovary (CHO) cells and CHO cells that stably express PKR2 (CHO-PKR2), cells were grown in α-MEM, transfected with the same method as for HEK293 and NIH3T3 cells. 42-hr after transfection, cells were treated with increasing concentrations of purified recombinant mouse PK2 (0-50 nM) for 6 hrs and then lysed. Luciferase activities were normalized to protein concentration.

In FIG. 2b, the letters C and B indicate CLOCK and BMAL1, respectively. The luciferase activity of PK2.8-Luc in the absence of C and B was designated as 1 unit. The results shown in FIG. 2b indicate that CLOCK and BMAL1 together, but neither alone, produced a strong increase in transcriptional activity (172-fold). One proximal E-box, residing within 130 bp upstream of the putative PK2 transcriptional start site, was also transactivated by CLOCK:BMAL1 heterodimers. This CLOCK:BMAL1-dependent activation was abolished when the E-box sequence CACGTG was mutated.

The transcriptional activity of a 72 bp construct in which the four E-boxes and their immediate flanking sequence were linked together (PK4E-Luc) was examined. The construct was generated by subcloning annealed oligonucleotides PK4E5 and PK4E3 into a luciferase reporter vector containing SV40 promoter (pGL3-Promoter). A comparable construct with all four E-boxes mutated (PK4EM-Luc) was similarly constructed with oligonucleotides PK4E5M and PK4E3M. All four E-boxes were mutated in PK4EM-Luc. The luciferase activity of pGL3-Basic was designated as 1 unit. As shown in FIG. 2C, CLOCK and BMAL1 together (but neither alone) caused a substantial increase in transcriptional activity through these four E-boxes (162-fold). This CLOCK:BMAL1-dependent activation was reduced to background level of activation when all four E-boxes were mutated. These results indicate that transcriptional activation by CLOCK:BMAl1 heterodimers requires at least one E-box.

Using the 2.8 kb PK2 promoter, the effects of negative elements of the SCN clockwork were examined. Inhibition of CLOCK:BMAL1-mediated transcription from PK2.8-Luc by mPers and mCrys was examined. The luciferase activity of PK2.8-Luc in the presence of C and B was designated as 100%. As shown in FIG. 2d, it was observed that mPERs caused 40%-60% inhibition of CLOCK:BMAL1-induced transcription, while the inhibition mediated by mCRY1 and mCRY2 was close to completion. Taken together, these in vitro studies indicate that clock gene products can regulate PK2 transcription.

The role of PKR2 activation on PK2 gene expression was examined by stably expressing PKR2 in Chinese Hamster Ovary cells (CHO-PKR2) and transfecting CHO-PKR2 cells with the 2.8 kb PK2 promoter fused to luciferase reporter nucleic acid. To prepare the PK2 promoter containing constructs, a 2.8 kb 5′-flanking region of the mouse PK2 gene was subcloned into pGL3-BASIC vector to generate PK2.8-Luc. 170 bp 5′-flanking region of the mouse PK2 gene containing a single E-box was synthesized with four long oligonucleotides and subcloned into the pGL3-BASIC vector to generate PKO.2-Luc. A mutant 5′-flanking sequence with the E-box CACGTG was created similarly with mutant oligonucleotides (PK0.2M-Luc). The luciferase activity of PK2.8-Luc in CHO cells in the absence of PK2 treatment was designated as 1 unit.

As shown in FIG. 2e, activation of PKR2 by PK2 dose-dependently stimulates CLOCK:BMAL1-mediated transcription of the 2.8 kb PK2 promoter (4.0±0.4 fold, Mean±SEM, n=3). The EC50 values of PK2 were 7.8±2.9 nM (n=3), similar to EC50 values (7.3±1.5 nM, n=4) obtained from the calcium mobilization of PKR2 activation by PK2. In the absence of CLOCK and BMAL1, activation of PKR2 alone by PK2 treatment (50 nM for 6 hr) did not activate the transcription of the 2.8 kb PK2 promoter [luciferase activity: 1.0±0.11, and 1.14±0.09, for CHO and CHO-PKR2, respectively]. In the presence of CLOCK and BMAL1, the stimulatory effect of PKR2 activation on the transcription of the 2.8 kb PK2 promoter was abolished by mCRY1 and mCRY2. Thus, PK2 can activate its own transcription in the SCN via activation of PKR2 in clock gene products-dependent manner.

In FIG. 2b-2e, the value shown is the mean±SEM of three replicates from a single assay. The results shown were representative of at least three independent experiments.

In summary, this example shows that the mouse PK2 promoter can be regulated by CLOCK:BMAL1 heterodimers, mPERs, mCRY1 and mCRY2.

### EXAMPLE III

Altered PK2 Rhythm in Clock Mutant Mice

This example shows that rhythmic expression of PK2 mRNA is disturbed in Clock-deficient (Clk/Clk) and Cryptochrome-deficient (Cry/Cry) mice.

Genetic studies with mutant animals have provided insight into the mechanism of the mammalian molecular clock. Mice deficient in mCry1, mCry2, and mPer3 show subtle changes in circadian cycle length, but without causing arrhythmicity (Vitaterna et al., Proc. Natl. Acad. Sci. USA 96:12114-19 (1999); Thresher et al., Science 282:1490-94 (1998); Shearman et al., Mol. Cell Biol. 20:6269-75 (2000); and van der Horst et al., Nature 398:627-30 (1999)). Mutations in Clock, mPer1 or mPer2 genes result in a more severe circadian phenotype which includes arrhythmicity after long-term housing in constant darkness (DD)(Cermakian et al., EMBO J. 20:3967-74 (2001); Vitatema et al., Science 264:719-25 (1994); King et al., Cell 89:641-53 (1997); Bae et al., Neuron 30:525-36 (2001); and Zheng et al., Cell 105:683-94 (2001)). Mice with disrupted Bmall gene, or deficient in both mPer1 and mPer2 exhibit the most severe phenotype: they are arrhythmic immediately after placement in DD (Bae et al., Neuron 30:525-36 (2001); Zheng et al., Cell 105:683-94 (2001); and Bunger et al., Cell 103:1009-17 (2000)). Similar arrhythmicity also occurs in mice deficient in both mCry1 and mCry2 genes (Vitatema et al., Science 264:719-25 (1994); and van der Horst et al., Nature 398:627-30 (1999)). The cloning of the Tau gene, which encodes a casein kinase and whose mutation causes a shortened circadian cycle in a strain of hamster, has revealed the importance of post-transcriptional mechanisms for SCN clockwork (Lowrey et al., Science 288, 483-91 (2000)).

In contrast to SCN pacemaker clockwork, relatively little is known about the mechanism by which circadian pacemaker systems transmit timing information to control physiology and behavior (Reppert and Weaver, Ann. Rev. Physiol. 63:647-76 (2001); Allada et al., Ann. Rev. Neurosci. 24:1091-119 (2001); and Hardin, Genome Biol 1, REVIEWS1023 (2000)). In Drosophila, pigment dispersing factor, a neuropeptide, and takeout, a clock-controlled gene that appears to encode a secreted protein, have been demonstrated as mediators that transmit circadian activity rhythms (Renn et al., Cell 99:791-802 (1999); and Sarov-Blat et al., Cell 101:647-56 (2000)). In mammals, transplant studies have indicated that signals that mediate the rhythmic output of the SCN also appear to be secreted molecules (Ralph et al., Science 247:975-8 (1990); Silver et al., Nature 382:810-13 (1996); and Earnest et al., Science 283:693-5 (1996)), but their biochemical identities are unknown.

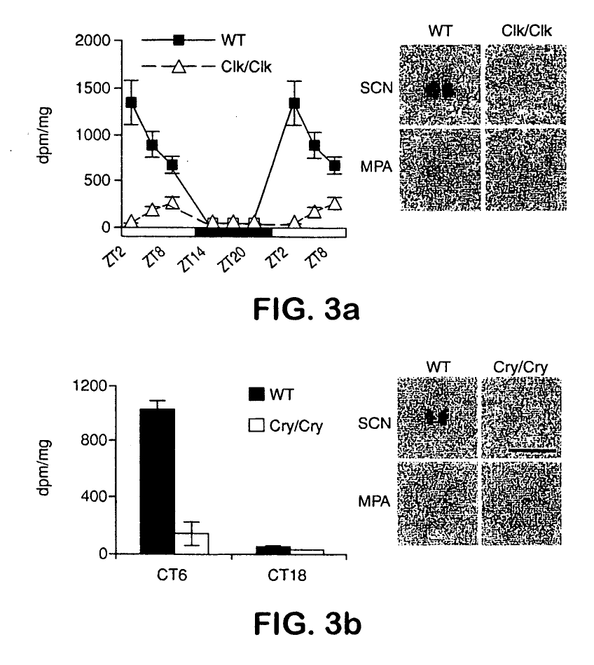

PK2 mRNA levels were examined in the SCN of mutant mice deficient in Clock (Clk/Clk)(Vitaterna et al., Science 264:719-25 (1994)) or Cryptochromes [(mCry1−/−, mCry2−/−), mCry-deficient] (van der Horst et al., Nature 398:627-30 (1999)). Methods for preparing sections through the SCN of Clk/Clk mice (on a BALB/c genetic background) and of mCRY-deficient mice (mCry1−/−, mCry2−/−, on a C57BL/6-129 hybrid background) and their respective littermate controls were obtained as previously described (Shearman et al., Science 288:917-24 (2000).; and Jin et al., Cell 96:57-68 (1999)). As shown in FIG. 3A, the circadian rhythm of PK2 mRNA in the SCN was observed to be severely blunted in Clk/Clk mutants under LD. The left graph in FIG. 3A depicts the temporal profiles of PK2 mRNA in SCN of Clk/Clk mice. Representative coronal sections (15 mm) of PK2 mRNA at ZT2 of wild type (WT) and Clk/Clk mice in SCN and MPA are shown on the right. Data for ZT2-8 are double plotted.

In Clk/Clk mice, PK2 mRNA was detectable only at ZT5-ZT8. The reduced peak level of PK2 mRNA in Clk/Clk mice was also observed to be shifted from about ZT4 to ZT8. It should also be noted that the PK2 mRNA levels in medial preoptic area did not change in Clk/Clk mice. These results show that the decrease in PK2 gene expression in Clk/Clk mice is specific for the SCN circadian clock. The severe blunting of PK2 circadian rhythm in Clk/Clk mice indicates that CLOCK has a positive regulatory effect on the expression of PK2 gene.

PK2 mRNA levels in the SCN of mCry-deficient mice also were examined. The left graph of FIG. 3b depicts the expression of PK2 mRNA in SCN of Cry/Cry mice at CT6 and CT18. Representative coronal sections of PK2 mRNA at CT6 of WT and Cry/Cry mice in SCN and MPA are shown on the right (scale bar=1 mm). Each value is the mean±SEM of 5-6 animals.

In contrast to the oscillation pattern seen in the SCN of wild type mice, PK2 mRNA levels were low at both CT6 and CT18 in mCry-deficient mice (FIG. 3b). PK2 mRNA levels in medial preoptic area were not observably different from wild type mice at both circadian times. The apparent low levels of PK2 at both circadian times in mCry-deficient mice appear unexpected as mCRYs are major inhibitors of CLOCK:BMAL1-mediated transcription (Kume et al., Cell 98:193-205 (1999)) and tonic mid-high mPer1 and mPer2 mRNA levels have been observed in mCry-deficient mice (Vitatema et al., Proc. Natl. Acad. Sci. USA 96:12114-19 (1999); and Okamura et al., Science 286:2531-34 (1999)). The immediate arrhythmicity of mCry-deficient mice in DD indicates the disruption of the functional circadian clock (van der Horst et al., supra).

In the absence of functional positive and negative limbs of the feedback loops, the absolute levels of PK2 mRNA likely depend on basal promoter activity and RNA stability. The essentially undetectable levels of PK2 mRNA in the SCN during the dark phase (FIG. 1b, c) suggest low basal PK2 promoter activity. It should also be noted that there are multiple copies of mRNA destability signal (AUUUA) in the 3′-untranslated regions of both the human and mouse PK2 mRNAs. These combined effects could contribute to the low levels of PK2 mRNA in the SCN in mCry-deficient mice. Taken together, these studies with mutant mice support that PK2 is a clock-controlled gene.

In summary, this example shows that mutant mice deficient in Clock (Clk/Clk) or Cryptochromes (mCry1−/−, mCry2−/−) have altered levels of PK2 mRNA in the SCN compared to wild type mice.

### EXAMPLE IV

Response of PK2 Rhythm to Light Entrainment

This example shows that PK2 expression is altered by light entrainment of mice.

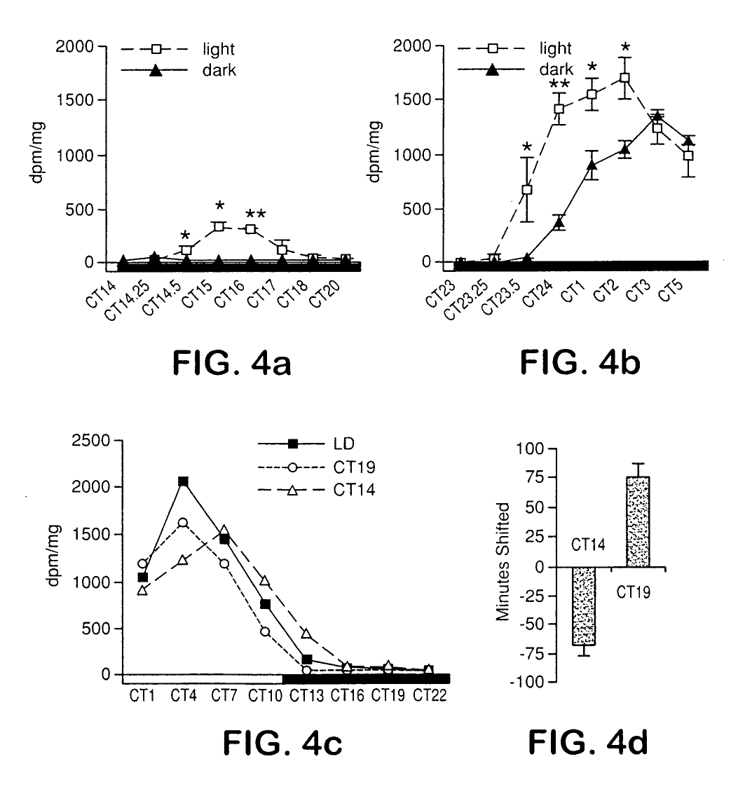

The PK2 mRNA level in the SCN in response to light pulses was examined. Animals were exposed to a 15 min light pulse at CT14 (FIG. 4a) or CT23 (FIG. 4b) on the first day in DD. Light pulses were 400 Lux for 15 min (FIG. 4a, b) and 400 Lux for 1 hr (FIG. 4c), respectively. In FIG. 4, open symbols and dashed lines denote light-exposed animals; closed symbols denote time-matched controls maintained in DD. Each value is the mean±SEM of 4-6 animals. Statistical analyses were performed using t-tests. *p<0.05, **p<0.01.

As shown in FIGS. 4a and 4b, PK2 mRNA levels increased rapidly and transiently 30 min after light exposure at both CT14 and CT23. At CT14, PK2 mRNA remained undetectable without light pulse. Following light exposure, PK2 mRNA levels increased by 30 min, peaked at 1-2 hr, and returned to control levels 4 hr after exposure. At CT23, PK2 mRNA levels rose with or without light pulse. However, in the presence of light pulse, PK2 mRNA levels increased more rapidly and robustly. PK2 mRNA returned to control levels 4 hr after exposure. At CT3, however, light pulse had no enhancing effect on PK2 mRNA levels. The light-inducibility of PK2 may result from activation of the CRE in the PK2 promoter (FIG. 2a).

The effect of light pulses that shift the phases of SCN-controlled behavioral rhythms on the phase of PK2 rhythm was also examined. Animals were exposed to 1-hr light pulse at CT14-15 or CT19-20 on the first day in DD. Control animals received no light pulse. In FIG. 4c, shaded and closed horizontal bars indicate light and dark periods under DD, respectively. FIG. 4d shows quantification of phase shifts (min, n=3) shown in FIG. 4c. The shift was measured at half-peak level of PK2 mRNA. By convention, delays are negative and advances are positive. One of the three experiments is shown.

As shown in FIGS. 4c and 4d, light pulses administered during early subjective night (CT14) cause about 70 min delay in the phase of PK2 expression. Similarly, light pulses administered during late subjective night (CT19) cause about 75 min advance in the phase of PK2 expression. These results indicate that light pulses that shift the phase of SCN-controlled behavioral rhythm alters the phase of PK2 expression. These light-induced shifts of PK2 rhythm correlate well with the shifts of locomotor rhythm.

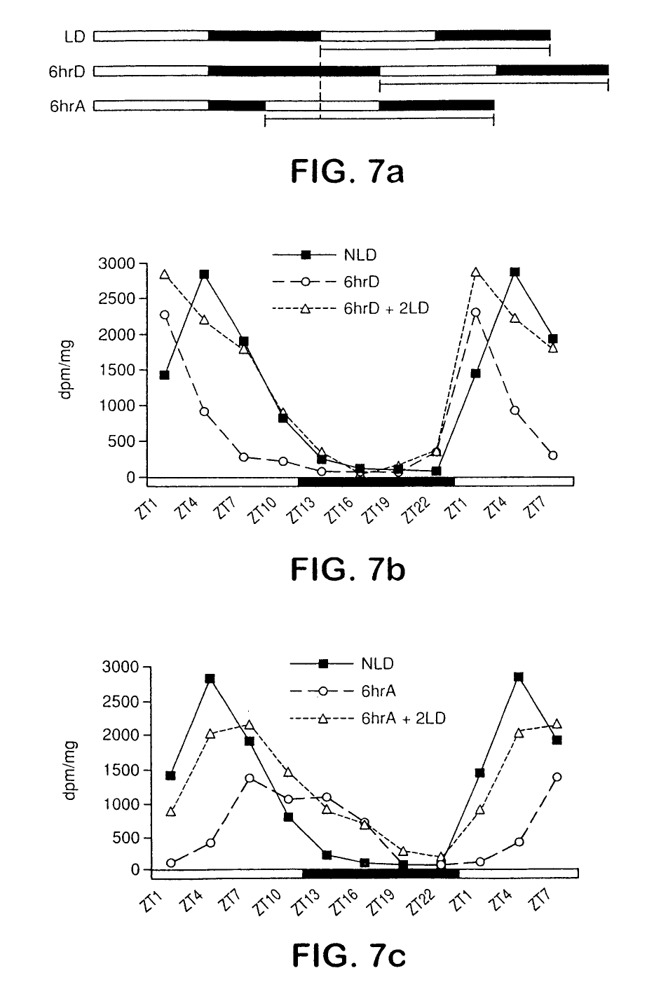

FIG. 7 shows PK2 mRNA expression in the SCN in response to abrupt shifts of light/dark cycle. Animals were entrained to 12 hours of light/12 hours of dark. The normal light/dark cycle (LD) was either delayed by 6 hours (6 hrD) or advanced by 6 hours (6 hrA). The periods of mouse brain samples are indicated by parentheses. Open and closed bars represent light and dark periods, respectively. FIG. 7b shows PK2 mRNA expression in normal LD, 6 hrD and 6 hrD +2 LD (6 hour delay followed by adaptation of 2 additional days). FIG. 7c shows PK2 mRNA expression in normal LD, 6 hrA and 6 hrA+2 LD (5 hour advance followed by adaptation of 2 additional light/dark cycles). Each value represents the average of 2 animals.

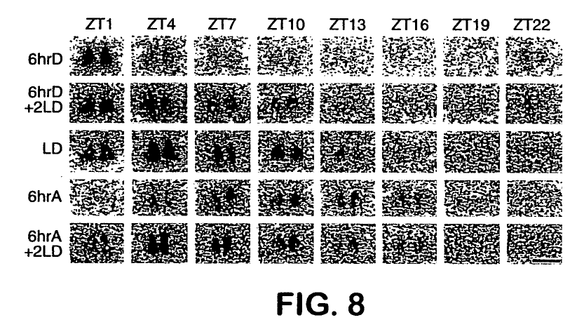

FIG. 8 shows temporal profiles of PK2 mRNA in the SCH in response to abrupt shifts of light/dark cycles. Animals were entrained to 12 hours light/12 hours day and subjected to either normal light/dark cycle (LD), 6 hour delay (6 hrD), 6 hour delay followed by adaptation of 2 additional light/dark cycles (6 hrD+2 LD), 6 hour advance (6 hrA), or 6 hour advance followed by adaptation of 2 additional light/dark cycles (6 hrA+2 LD). Representative images of coronal sections (20 μm) at the SCN levels are shown. Scale bar=1 mm.

In summary, this example shows that PK2 rhythm in SCN responds to light entrainment.

### EXAMPLE V

Expression of PK2 Receptor in SCN Output Targets

This example shows PK2 receptor expression in the mouse brain.

Expression of PK2 receptor mRNA in male adult C57B16 mouse brain was examined by in situ hybridization. FIG. 5, top panel (a-e), represents autoradiographic images of PKR2 mRNA in lateral septum (LS), paraventricular thalamic (PVT) and hypothalamic nucleus (PVN), suprachiasmatic nucleus (SCN), paratenial nucleus (PT), paracentral nucleus (PC), lateral habenula (LHb), dorsal medial hypothalamic nucleus (DMH) and arcuate nucleus (Arc). FIG. 5, middle panel (f-j), depicts dark field microscopic images of PKR2 mRNA from the boxed regions in top panel (a-e). FIG. 5, bottom panel, depicts microscope images of PK2 (k,l) and PKR2 mRNA (m, n) in SCN at high magnification. Both dark field (k,m) and cresyl-violet stained sections (l,n) are shown. For top panel, scale bar=2 mm; for middle and bottom panels, scale bar=0.5 mm. All images were obtained from mouse brains collected at ZT1.

It was found that PKR2 mRNA is abundantly expressed in the SCN, paraventricular nucleus of hypothalamus (PVN), dorsal medial nucleus of hypothalamus (DMH), paraventricular and paratenial nuclei of thalamus (PVT/PT), paracentral thalamic nucleus (PC), lateral habenular nucleus (LHb) and lateral septal nucleus (LS). As shown in FIG. 6, PKR2 mRNA is also moderately expressed in lateral globus pallidus (LGP), amygdala and other regions.

The highest expression of PKR2 was found in PVT. Intriguingly, most of the PKR2 mRNA-positive nuclei are the primary target areas of SCN output pathway (Klein et al., New York: Oxford Univ. Press. 467 pp. (1991); Moore, Ann. Rev. Med. 48:253-66 (1997); Sofroniew and Weindl, Amer. J. Anat. 153:391-429 (1978); Watts et al., J. Comp. Neurol. 258:204-29 (1987); Watts and Swanson, J. Comp. Neurol. 258:230-52 (1987); Leak and Moore, J. Comp. Neurol. 433:312-34 (2001); and Buijs, Prog. Brain Res. 111:229-40 (1996)). The high expression of PKR2 in these primary target areas of SCN output indicates that PK2 could serve as a signaling molecule mediating SCN output. PKR2 mRNA is also extensively expressed in the SCN (FIGS. 5c, h, m). In contrast to the robust rhythm of PK2 mRNA, PKR2 mRNA in the SCN and other PKR2-positive nuclei is non-oscillating. High magnification microscopic images show that most neurons within the SCN express both PK2 and PKR2 mRNAs (FIGS. 5k-n). This expression pattern of PK2 and PKR2 mRNA in the SCN indicates that the PK2/PKR2 system could play a role in synchronizing SCN output.

### EXAMPLE VI

PK2 Administration Alters Circadian Locomotor Activity

This example shows that PK2 administration to rats alters their circadian locomotor activity.

To directly assess whether PK2 mediates the circadian behavioral output, the effects of intracerebroventricular (icv) injection of recombinant PK2 on wheel-running behavior was examined. Recombinant human PK2 was produced and purified as described previously (Li et al., Mol. Pharm. 59;692-8 (2001)). Male Sprague-Dawley rats (300-350 g) were used to assess pharmacological effects of PK2 on locomotor rhythms. A 23-gauge guide cannula was implanted to the lateral ventricle (0.4 mm rostral to bregma, 3.0 mm ventral to dura, 2.4 mm lateral to midline, Charles River Laboratory). A 30-gauge stylet was placed in the guide cannula to maintain patency. Rats were housed in running-wheel cages for two weeks in LD, placed in DD for 2-5 days and then subjected to icv injection of recombinant PK2 at CT14. During icv injections, the stylet was removed and a 30-gauge injector attached to a Hamilton syringe by plastic tubing was inserted. Each animal received either 5 μl (1 mg/ml) of PK2 or saline (under dim red light <1 lux) over a 2-minute period. Animals were immediately returned to their cages and wheel-running activities were monitored in constant darkness with a PC system (Colbourn Instrument, Allentown, Pa.). Running-wheel data were analyzed at 5 minute intervals. Inhibition of nocturnal wheel-running was measured as activity counts during CT15-CT24, and expressed as a percentage of average counts obtained from the same time intervals of two days prior to treatment. Activation of wheel-running in subjective day in PK2-treated rats was measured as activity counts during CT0-CT12, and expressed as a percentage of average counts obtained from two nights (CT13-CT24) prior to treatment.

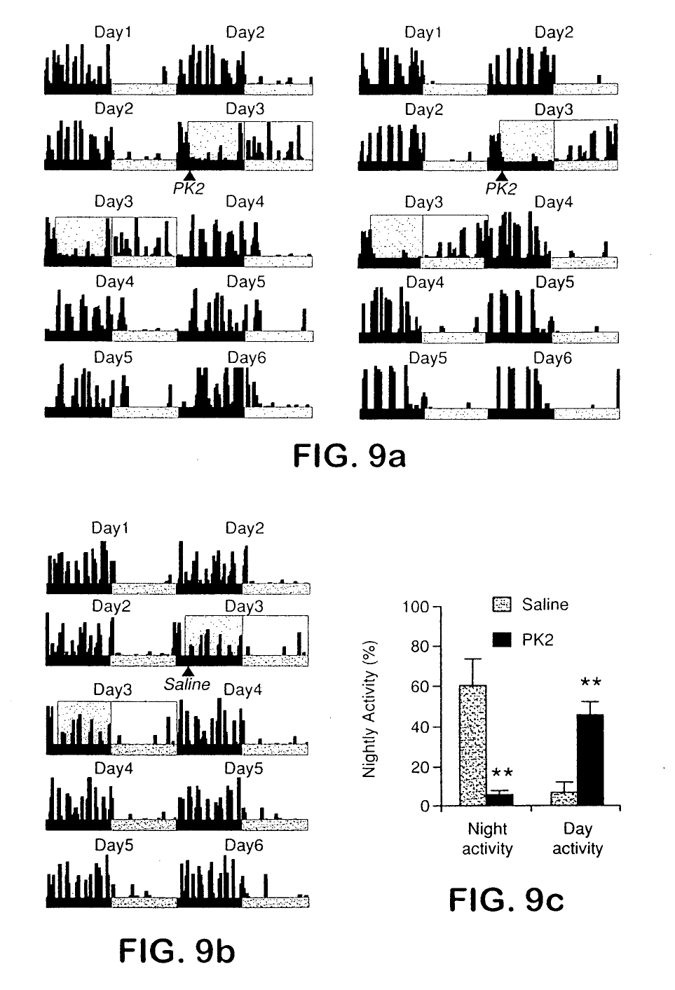

FIGS. 9a and 9b show representative actograms of rats injected with PK2 or saline at CT14. Shaded and closed horizontal bars indicate subjective light and dark periods under DD, respectively. The highlighted areas indicate night locomotor activity after treatment. The boxed areas represent locomotor activity during subjective day following treatment. FIG. 9c shows quantification of night and day locomotor activity following delivery of PK2 (n=7) or saline (n=6). The effect of PK2 on wheel-running activity during night phase (CT15-CT24) and day phase (CT0-CT12) was expressed as a percentage of nightly activity. Statistical analyses were performed using t-tests (**p<0.01).

The results shown in FIG. 9 indicate that PK2 delivery suppressed the high wheel-running behavior associated with dark phase, while administration of saline only slightly inhibited wheel-running behavior. In contrast to the normally quiescent daytime activity, PK2-treated rats were about half as active during day as at night. PK2 infusion caused slight delay of wheel-running rhythm in subsequent days, however, this delay was not significantly different from controls. These results indicate that PK2, which is normally high during subjective day, inhibits wheel-running activities. During subjective night when endogenous PK2 levels are low, the wheel-running activities are disinhibited. The increased wheel-running activity observed on the next subjective day in PK2-treated rats could be the results of PKR2 receptor desensitization caused by PK2 administration. Thus, PK2 is an output molecule that transmits the circadian locomotor rhythm of the SCN clock.

In summary, this example shows that PK2 administration to rats alters their circadian rhythm.

### EXAMPLE VII

Measurement of Cytosolic Free Calcium

This example describes an assay for measurement of cytosolic free calcium.

Cells were suspended in HEPES medium and incubated with 2 μM of fura-3 AM for 20 min at 31° C. The cells were then centrifuged, washed, resuspended in fura-3 free medium and seeded into 96 wells at 4×10⁴ cells per well. The cells were loaded with Fluo-3 AM (Molecular Probes) in standard buffer solution (130 mM NaCl, 2 mM CaCl₂, 5 mM KCl, 10 mM glucose, 0.45 mM KH₂ PO4, 0.4 mM Na₂ HPO₄, 8 mM MgSO4, 4.2 mM NaHCO₃, 20 mM HEPES and 10 μM probenecid) with 0.1% fetal bovine serum for 1 h at 37° C., then washed with a standard buffer solution. Transient changes in [Ca2+]ᵢ evoked by prokineticins were monitored using the FLIPR system (Fluorometric Imaging Plate Reader; Molecular Devices) at 488 nm for 210 s.

### EXAMPLE VIII

Temporal Profiles of PK2 mRNA in the SCN in Response to Light/Dark Cycles

This example shows that abrupt shifting of light/dark cycles resulted in altered PK2 molecular rhythm in the SCN.

Light/dark shift experiments were performed using adult C57B16 mice (Taconic Farms, N.Y.), which were entrained under 12 hour light: 12 hour dark (12L:12D, lights on 7:00 am, off 7:00 pm) cycle for two weeks with food and water available ad libitum. Light phase was either delayed by 6 hours (lights on 7:00 am, off 1:00 am) or advanced by 6 hours (lights on 7:00 am, off 1:00 pm) and samples were taken every three hours for the 24 hour period (Zeitgeber time, ZT, ZT1-22). To test the adaptations of these shifted mice, animals were placed for an additional two normal light/dark cycles and samples were collected for another 24 hour period.

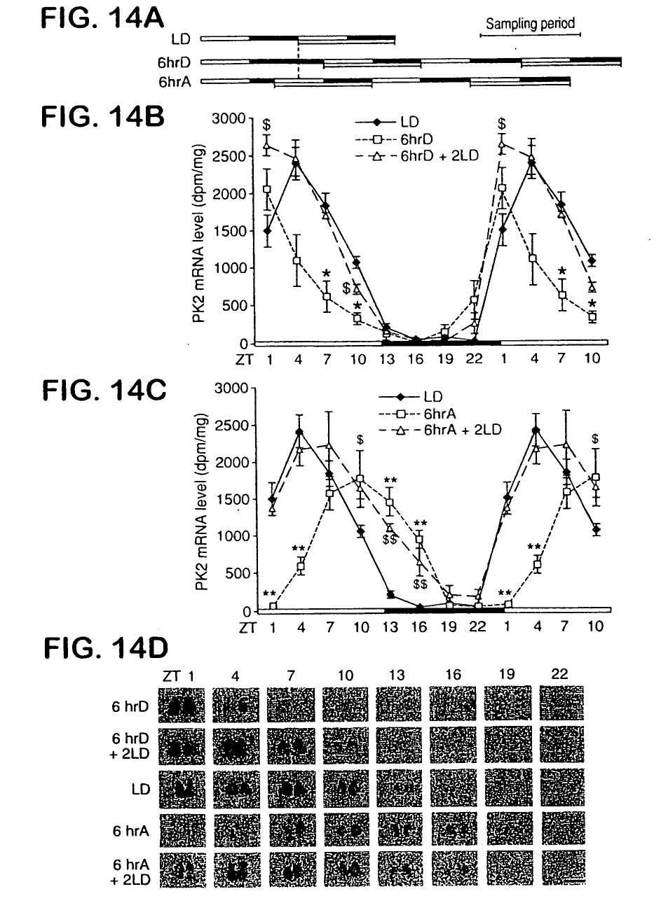

FIG. 14 shows the results of experiments in which PK2 mRNA rhythm in the SCN was measured for a 24 hour cycle. FIG. 14A shows a diagram of the light and dark period to which animals were exposed. As shown, animals were first entrained under normal 12 hour light: 12 hour dark (LD) cycle, followed by either 6-hour delay (6 hrD) or 6-hour advance (6 hrA) of light phase. FIGS. 14B and C show PK2 mRNA levels under conditions of 6 hour delay or 6 hour advance, respectively. Both 6-hour delay and 6-hour advance caused obvious changes in PK2 mRNA oscillation rhythm when compared to normal LD cycle, as indicated in FIGS. 14B and C. However, there were significant differences between these regimens. In 6-hour delay, the pattern of PK2 mRNA expression was similar to that observed under normal LD, with a slightly reduced PK2 peak level, and its rhythm advanced by about 3 hours (FIG. 14B). Similar to normal LD, PK2 mRNA was expressed during light period and remained low during dark phase. However, an early onset of PK2 mRNA at ZT22 was observed. In contrast, advance of 6-hour caused more changes in PK2 mRNA rhythm. The peak of PK2 mRNA was reduced and delayed by about 6 hours (FIG. 14C). Unlike normal LD, the peak of PK2 mRNA delayed to about ZT10 and the level of PK2 remained high during the first half of the dark period. The adaptation of these shifted mice was further tested for two additional LD cycles. As judged by the rhythm of PK2 mRNA expression in the SCN, mice under 6-hour delay regimen adapted quickly after placement in 2 additional LD cycles (FIG. 14B). There was also clear adaptation in 6-hour advanced mice, however, PK2 mRNA rhythm was still significantly different from control groups (FIG. 14C).

Taken together, these results indicate that a 6-hour advance significantly causes more changes in the molecular rhythm of PK2 in the SCN, and that mice adapt more quickly to the 6-hour delay than to the 6-hour advance regimen.

In FIGS. 14B and C, open and filled horizontal bars indicate light and dark periods, respectively. Abbreviations are as follows: normal light/dark cycle (LD); 6-hour delay (6 hrD); 6-hour delay followed by adaptation of 2 additional LD (6 hrD+2 LD); 6-hour advance (6 hrA); and 6-hour advance followed by adaptation of 2 additional LD (6 hrA+2 LD). In FIG. 14D, which shows representative autoradiograms depicting PK2 mRNA expression in the SCN under different light/dark conditions, the scale bar=1 mm, and each value is the mean±SEM of 3 animals. * is p<0.05, and ** is p<0.01 (Student's t-tests), which represent significant differences from control levels under LD for 6 hrD and 6 hrA. $ p<0.05, and $$ p<0.01 represent significant differences from control levels under LD for 6 hrD+2 LD and 6 hrA+2 LD.

In summary, this example shows that in mice exposed to abrupt shifting of light/dark cycles, PK2 mRNA expression was altered.

### EXAMPLE IX

Molecular Rhythm of PK2 in mCry1,2−/− Mice

This example shows that under normal light/dark conditions, some molecular rhythms, including a PK2 rhythm, are preserved in clockwork mutant mice.

Light regulated mPer2 molecular rhythm in the SCN of cryptochrome-deficient (mCry1,2−/−) mice that lack functional circadian clock has been observed to be preserved. To determine if light-regulated PK2 molecular rhythm in the SCN is preserved in mCry1,2−/− mice, levels of PK2 mRNA were observed in mCry1,2−/− mice under both light/dark (LD) and constant dark (DD) conditions.

In these experiments, wild type and mCry1,2−/− mice were entrained to normal 12L: 12D and sampled every three hours for the 24 hour period (Zeitgeber time, ZT, ZT1-22). A second group of mCry1,2−/− mice were placed into two days of constant darkness (2DD) (Circadian time, CT, CT 1-22). For light pulse experiments, mCry1,2−/− mice received a 15 min light pulse (400 lux) at ZT14, and sampled one or two hours after light pulse. Dark control mCry1,2−/− mice did not receive a light pulse.

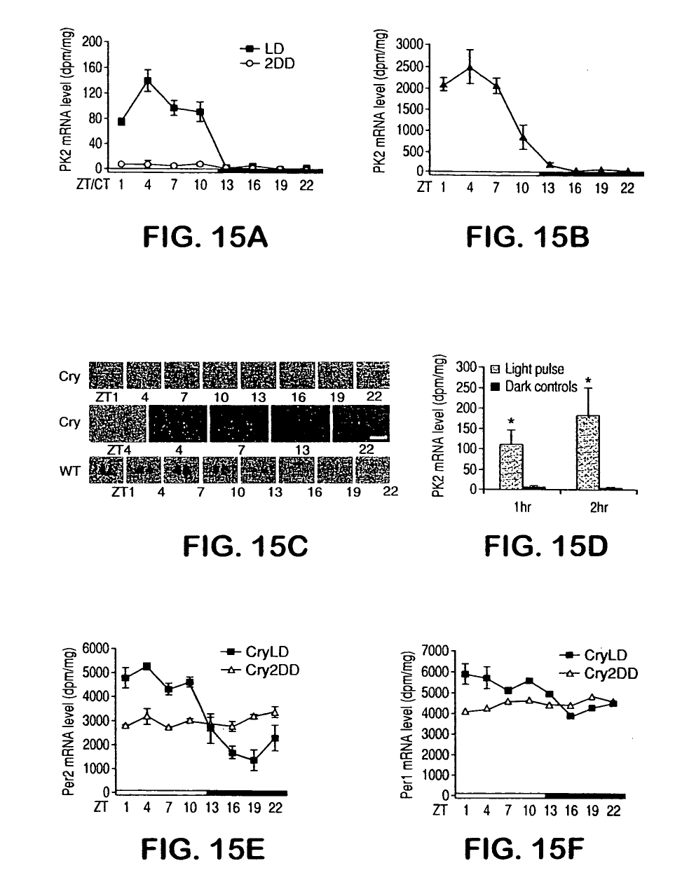

To determine PK2 molecular rhythm in the SCN of mCry1,2−/− mice, levels of PK2 mRNA were determined. PK2 mRNA expression in mCry1,2−/− mice is shown in FIG. 15A, while PK2 mRNA expression in wild type mice under 12L:12D (LD) is shown in FIG. 15B. FIG. 15C shows representative autoradiograms of PK2 mRNA in mCry1,2−/− mice and wild type mice under normal LD are shown (top and bottom row, respectively). Representative dark field images of PK2 mRNA in mCry1,2−/− mice under LD are shown in the middle row. The bright field image shows a cresyl-violet stained section, which depicts the cellular structure of SCN. In FIG. 15C, the scale bar=1 mm. Each value represents the mean±SEM of 3-6 animals. For FIG. a6D, PK2 mRNA levels in wild type and mCry1,2−/− mice are shown under light pulse and dark control conditions. In this figure, * p<0.05, Student's t-tests. The results shown in FIG. 15D indicate that PK2 mRNA level increased one and two hours in mCry1,2−/− mice after delivery of a brief light pulse at ZT14.

PK2 molecular rhythm in the SCN of mCry1,2−/− mice was apparent under normal LD, with the presence of a low level of PK2 mRNA in the light phase and absence of PK2 mRNA in the dark phase, as indicated in FIGS. 2A and C. Similar to wild type mice, the peak level of this residual PK2 rhythm was around ZT4, although its magnitude was smaller (only about 5% of that observed in wild type mice). This residual PK2 molecular rhythm was absent when mCry1,2−/− mice were placed under constant darkness (FIG. 1 SA). These results indicate that the residual PK2 rhythm observed in mCry1,2−/− mice under normal LD is directly driven by light.

The temporal profile of mPer1 and rnPer2 rhythm in mCry1,2−/− mice under normal LD also was determined (FIGS. 15, E and F). The molecular rhythm of mPer2 was observed to remain intact, with about 4-fold higher levels during the light phase than the dark phase (FIG. 15E), as has been observed in wild type animals. This molecular rhythm of mPer2 was not observed under constant darkness (FIG. 15E). A diurnal rhythm also was observed for mPer1 in mCry1,2−/− mice under normal LD, but not DD (FIG. 15F). Thus, mPer1 and mPer2 were non-oscillating and slightly elevated during the dark phase of mCry1,2−/− mice under constant darkness (FIGS. 2, E and F). These results indicate that under normal light/dark conditions, some molecular rhythms, including a residual PK2 rhythm, are preserved in clockwork mutant mice. This residual PK2 rhythm could contribute to the maintenance of behavioral rhythms observed in mCry1,2−/− mice under normal LD.

To obtain the results shown in FIG. 15, antisense and sense riboprobes containing the coding region of mouse PK2 (accession number AF487280 1-528 nt), Per1 (accession number AF022992 340-761 nt) or Per2 (accession number AF035830 9-489 nt) were generated. In situ hybridizations were processed as described (Winzer-Serhan et all, 1999, Cheng et al., 2002). Briefly, brains were quickly removed, frozen in isopentane and stored at −70° C. until use. For all experiments, 20 mm sections were collected and mounted onto superfrost-plus slides. Tissue sections were fixed in 4% paraformaldehyde for 1 hour followed by three 0.1M PB washes. Sections were pretreated with proteinase K (1 mg/ml), acetylated, dehydrated and air-dried. Sections were hybridized with riboprobes (antisense or sense for PK2, Per1 or Per2, 1×10⁷ cpm/ml) and incubated at 60° C. for 18 hours, followed by RNAase (20 mg/ml) digestion, decreasing salinity washes and a 30 minute high stringency (68° C.) wash. After dehydration and air-drying, tissue sections were exposed to Kodak Biomax film and autoradiograms were developed after 3-4 days. Some of the issue sections were dipped in liquid NTB-2 emulsion and developed after five weeks of exposure period. Emulsion-dipped sections were counterstained with cresyl violet, and cover-slipped for analysis by transillumination microscopy. Specific hybridization signals of PK2, Per1 and Per2 were quantitatively analyzed using a video-based computer image analysis system (MCID, Imaging Research, St. Catharine's, Ontario, Canada). A calibration curve of optical density versus radioactivity (dpm/mg tissue wet weight) was constructed using ¹⁴C-standards. Specific hybridization signals in the SCN were determined by subtracting background values from adjacent brain regions that have no hybridization signals. Data were normalized with respect to the differences between signal intensities in same areas of SCN. Images of SCN were taken from autoradiograms and prepared in Adobe Photoshop. Emulsion-dipped images were taken under transillumination microscope (BX50, Olympus) using Spot camera software version 2.2.2 (Diagnostic Instruments, Sterling Heights, Mich.). Captured images were transferred to Adobe Photoshop 6.0 for figure preparation.

### EXAMPLE X

Light Inducibility of PK2 in Melanopsin-Deficient Mice

This example shows that melanopsin is an input mechanism for the light inducibility of PK2.

Melanopsin is the primary photoreceptor for circadian photic entrainment. To determine if the light inducibility of PK2 is blunted in melanopsin-deficient mice (Opn4−/−), PK2 mRNA expression in the SCN of Opn4−/− mice was observed.

Wild type and melanopsin-deficient (Opn4−/−) mice (on C57B16/129 hybrid background) were entrained to normal 12L;12D and sampled every three hours for the 24 hour period (Zeitgeber time, ZT, ZT1-22). For light pulse studies, wild type and Opn4−/− mice received a 15 min light pulse (400 lux) at ZT14 and were sampled one or two hours after light pulse. PK2 mRNA expression was measured one or two hours after the delivery of the 15-min light pulse at ZT14.

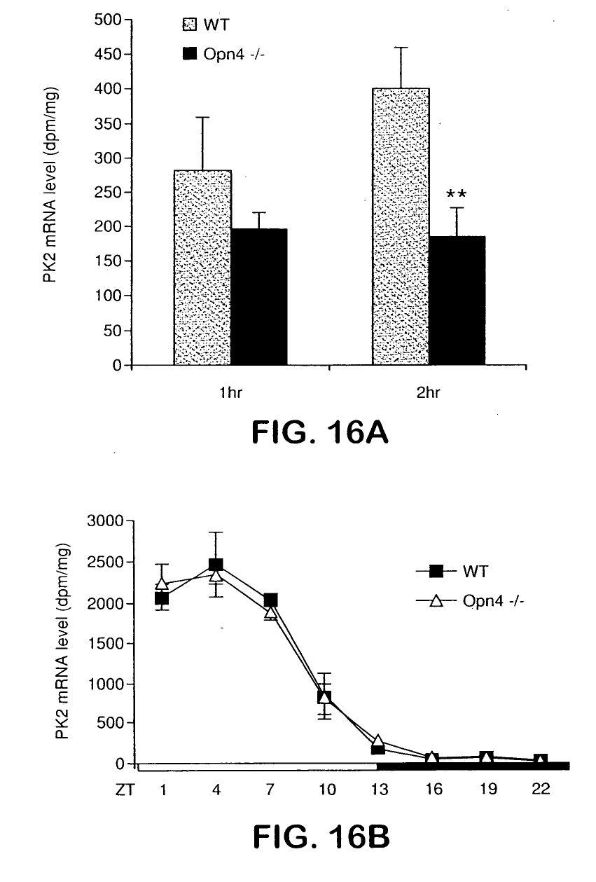

The results of these experiments are shown in FIG. 16A. Shaded and filled bars represent light pulse-induced PK2 mRNA in wildtype (WT) and Opn4−/− mice, respectively. As shown, light pulse-induced PK2 mRNA in Opn4−/− mice was reduced by about 30% and 50%, one and two hours after light pulse, respectively.

As shown in FIG. 16B, the temporal profile of PK2 mRNA in wildtype (filled squares) and Opn4−/− mice (open triangles) under normal LD cycle also was examined. The oscillation profile of PK2 in the Opn4−/− mice was similar to that observed in the wild type mice, consistent with the normal locomotor behavior in Opn4−/− mice under light/dark cycle. In FIG. 16B, each value represents the mean±SEM of 4 animals. ** p<0.01, Student's t-tests.

These results indicate that melanopsin is an input mechanism for the light inducibility of PK2, but that one or more other light sensing mechanism of light entrainment are also involved.

### EXAMPLE XI

The Role of PK2 Expression in Encoding Photoperiodic Information

This example shows the effect of photoperiod on PK2 mRNA rhythm in the SCN.

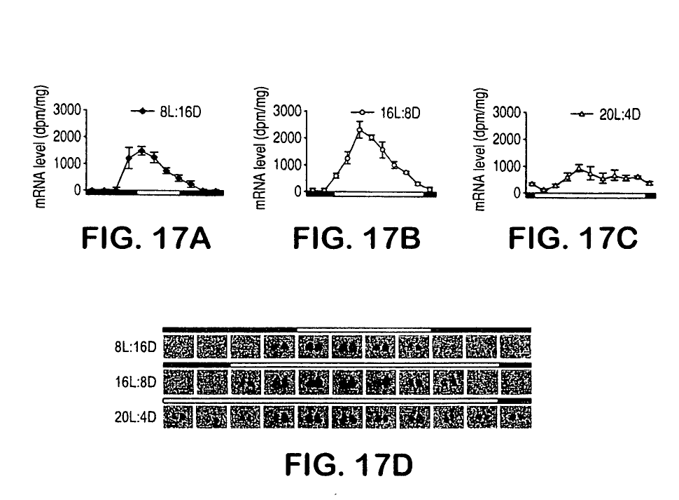

To determine if the molecular rhythm of PK2 in the SCN encodes photoperiodic information, animals were entrained under normal 12 hour light: 12 hour dark (LD) cycle, followed by placement in different photoperiod for four weeks: 8 hour light: 16 hour dark (8L:16D), 16 hour light: 8 hour dark (16L:8D) or 20 hour light: 4 hour dark (20L:4D). For the 20 hour light: 4 hour dark (20L:4D), animals were first placed in 14L: 10D for one week, transferred to 16L:8D for another week, followed by two weeks in 20L:4D. Samples used for determining PK2 mRNA levels in the SCN were taken every two hours throughout the 24 hour cycle. The results of this study are shown in FIG. 17. Open and filled bars indicate light and dark periods, respectively.

FIG. 17A shows PK2 mRNA levels under 8L: 16D conditions. FIG. 17B shows PK2 mRNA levels under 16L: 8D conditions. FIG. 17C shows PK2 levels under 20L:4D conditions. FIG. 17D shows representative autoradiograms of PK2 mRNA in the SCN under the different photoperiods described above. Scale bar=1 mm. Each value represents the mean±SEM of 3-4 animals.

As indicated in FIGS. 17C and D, when animals were entrained to an even longer light period (20L:4D), PK2 mRNA also followed the light period and was detected during the entire 20 hour light period. During the short photoperiod (8L: 16D), however, PK2 mRNA rises before lights on and persists after lights off, as shown in FIG. 17A. The temporal profile of PK2 mRNA in the short photoperiod is similar to that observed in the normal LD or constant darkness (2DD). Thus, for shortened light periods, the circadian loops have dominant control over PK2 rhythms. Taken together, these results indicate that photoperiodic information likely is encoded by the duration of PK2 expression.

Interestingly, the peak of PK2 mRNA expression is higher in long day (16L:8D) than in short day (8L;16D) or normal LD. However, a further increase in light period (20L;4D) depressed the peak PK2 level (FIG. 17C).

As shown in FIG. 17B, with increased photoperiod length (16L:8D), PK2 mRNA expression expanded to the entire 16 hour light phase, in comparison to the results shown in FIG. 16B, which indicated that during normal LD cycle, PK2 mRNA was highly expressed during the 12-hour light phase with peak level at ZT4.

In summary, this example shows that PK2 mRNA expression is regulated by photoperiod.

### EXAMPLE XII

Distribution of the Prokineticins and Prokineticin Receptors in the Rhesus Monkey Brain

PK2 expression oscillates during the day/night cycles in the rodent brain and acts as the key, central signaling molecule transmitting circadian activity/rest rhythm in nocturnal animals. The known similarities in the expression of clock and other genes in nocturnal and diurnal animals strongly suggested that the role of PK2 in modulating circadian rhythm-controlled behaviors in diurnal animals would be similar to that observed in the nocturnal rodent. For example, the oscillating phase of per1 is similar in diurnal animals (sheep and ground squirrels) and nocturnal rodents (Lincoln et al., Proc Natl Acad Sci USA. 99:13890-5, (2002); Mrosovsky et al. J Biol Rhythms 16:471-8, (2001)). Given the importance of targeting PKR2 for drug discovery purposes, it is crucial to demonstrate directly the role of PK2 plays in the regulation of circadian rhythms of diurnal animals, in particular, non-human primates.

This study confirms the oscillation of PK2 in rhesus monkey brain and demonstrates that the oscillatory phase of PK2 is the same in monkey as in nocturnal rodents, i.e., PK2 levels are high during daytime and low during nighttime. Thus, these results are consistent with the prediction based on the expression pattern of other clock genes.

This study also shows that PKR2 is expressed in major SCN target areas, including PVT, PVN, LS, DMH, and BST (see abbreviations, below). However, differences in the expression of PK2 and PKR2 in monkey vs. mouse brains are observed.

PKR2 is strongly expressed in the dorsal raphe N. (reticular activation system) and superior colliculus (visual input and output) in the monkey but not the mouse. Thus, activation of PKR2-positive neurons in these primary targets may modulate the sensory/motor functions in the monkey. In addition to the SCN, PK2 is also expressed in primary SCN targets (i.e., PKR2-positive regions) in the monkey, but not in the corresponding areas in the mouse brain. Since PK2 has been shown to stimulate the firing of PKR2-positive neurons (Cottrell et al., 2004), it is likely that a positive feedback mechanism operates in primary SCN targets in the monkey. This effect may explain differences between the function of the SCN derived signals in nocturnal vs. diurnal animals.

In summary, the following expression studies further validate PKR2 as a drug target for sleep/wake and other circadian-rhythm disorders. The difference in the expression of PK2 and PKR2 in monkey brain vs. mouse brains highlights the importance of conducting future pharmacologic or behavioral studies in the monkey or other diurnal species to support drug discovery and development efforts.

Methods: The rhesus monkeys used in this study were obtained in collaboration with Dr. Henryk Urbanski, Oregon Primate Center. Monkeys were sacrificed at various times of the light/dark cycle under carefully controlled conditions. Specific probes for monkey PK2, PK1 and PKR2 and PKR1 were designed.

Animals: Six young adult female monkeys were used. Monkeys were subjected to the standard 12 hr light: 12 hr dark (LD) cycle in the facility of Oregon Primate Center (with light on 7 am and light off 7 pm). At the time of sacrifice, monkeys were deeply anesthetized with Nembutal, and brought into the dissection room with their eyes covered with a dark cloth to prevent exposure to light. Blood samples were drawn from femoral veins and plasma levels of melatonin were quantified by radioimmunoassay with a commercially available kit.

The melatonin levels in the six animals are shown in Table 1.

TABLE 1
Serum melatonin levels.

| | Age | | Serum |
|---|---|---|---|
| Animal ID | Years/days | Time of sacrifice | melatonin (pg/ml) |
| 21618 | 10/37 | 11 am | <0.5 |
| 21644 | 8/106 | 3 pm | <0.5 |
| 21355 | 7/85 | 7 pm | 8.0 |
| 21670 | 12/133 | 11 pm | 37.4 |
| 20528 | 11/89 | 3 am | 14.5 |
| 20572 | 8/70 | 7 am | 6.0 |

Monkeys were transcardially fixed with 4% paraformaldehyde. Brains were dissected from the skull and separated into three blocks (hypothalamus/thalamus, cortex, and brain stem) for micro-sectioning.

An additional female monkey was sacrificed at about 9 am (ID 15452). This animal was handled similarly.

In situ hybridization: Twenty five micrometer sections were cut on a cryostat and serial sections were collected. Riboprobes were generated by T7 or SP6 RNA polymerases and were radioactively labeled with ³⁵S-UTP, and were used at a concentration of 1×10⁷ cpm/ml. Tissue sections were pretreated with proteinase K, hybridized for 18 hours at 60° C., followed by RNAse digestion, high stringency washes and dehydration. Tissue sections were then exposed to BioMax film (Kodak) for 7-21 days. For SCN sections, specific hybridization signals were quantitatively analyzed by comparing to ¹⁴C-standards of known radioactivity using a video-based computer image analysis system (MCID, Imaging Research, St. Catharine's, Ontario, Canada). A calibration curve of optical density versus radioactivity (dpm/mg tissue wet weight) was constructed using ¹⁴C-standards. Figures were prepared by using Adobe Photoshop (version 5.5)

Results: The results obtained for the expression patterns of mRNAs are presented separately for PK2, PKR2, PK1 and PKR1:

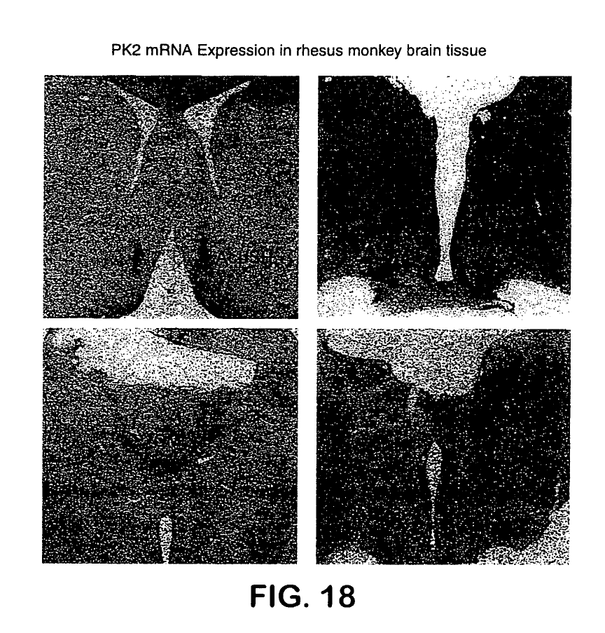

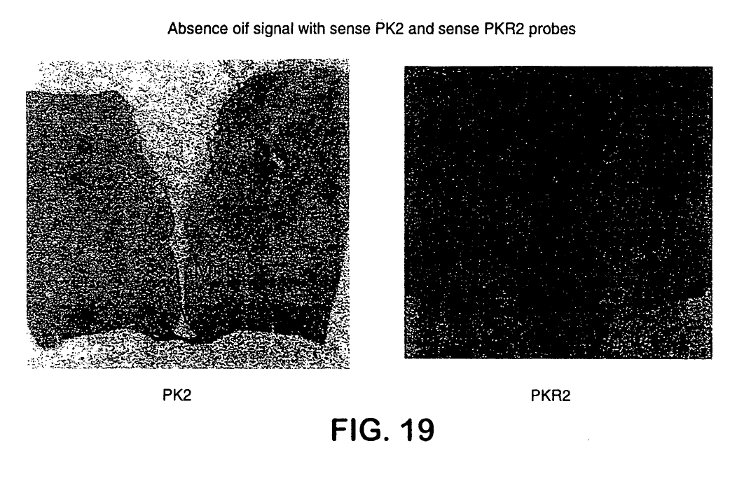

PK2: PK2 mRNA is strongly expressed in the SCN (FIG. 18). Sense probes for PK2 gave a low background (FIG. 19).

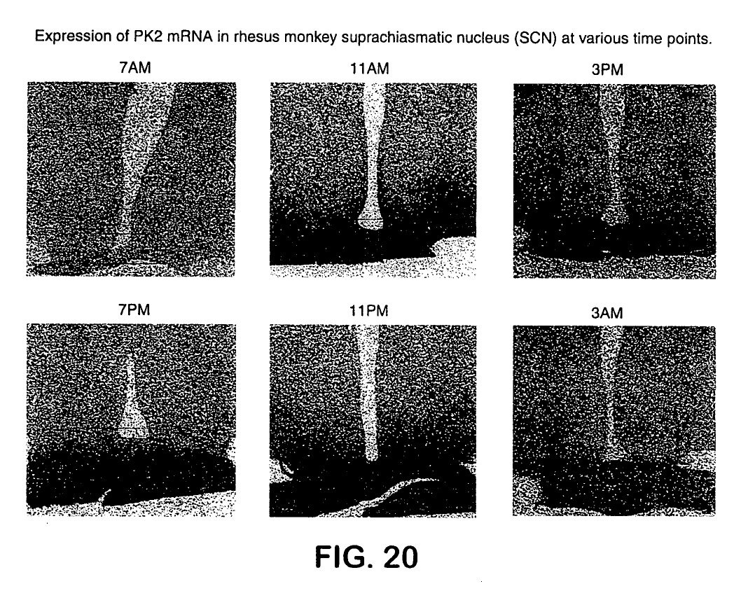

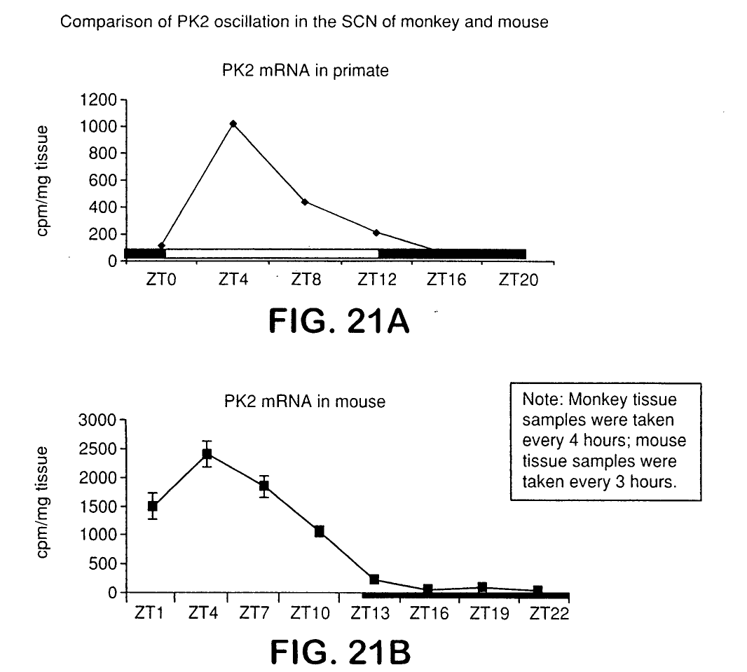

As previously observed in the mouse brain, PK2 mRNA expression oscillates in the SCN with high levels during daytime and low, or negligible, levels at nighttime. Similar results are found in the monkey SCN (FIGS. 20 and 21).

PK2 oscillation in the SCN has a similar time course in the monkey as previously described for the mouse SCN (FIG. 21A, B).

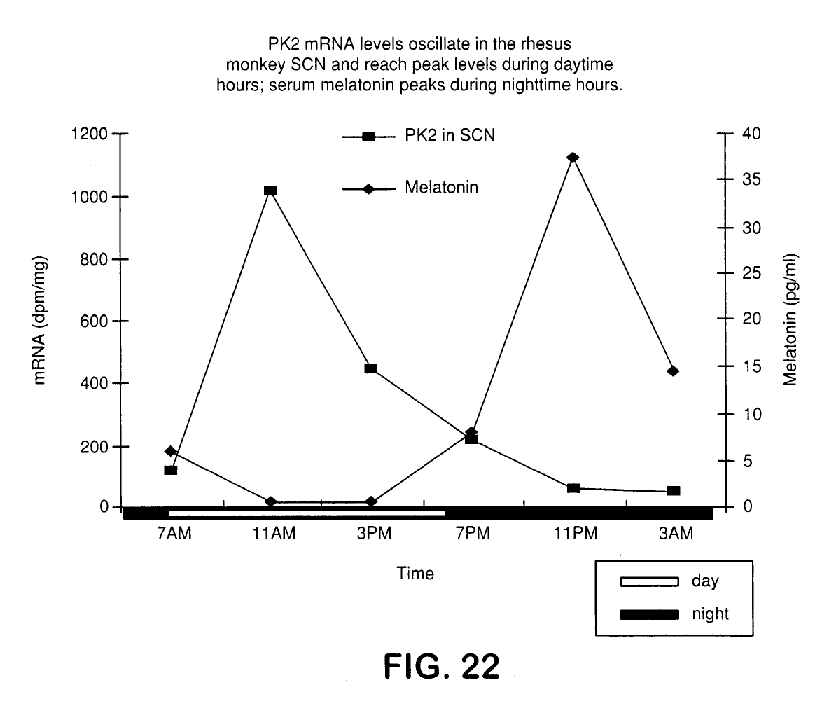

While PK2 levels in the SCN peak during daytime hours in the rhesus monkey, melatonin levels peak during the nighttime (FIG. 22).

In addition to the SCN, PK2 mRNA is expressed in other brain areas in the monkey (FIG. 19). Some of these areas of expression are also observed in the mouse; for example, PK2 expression is observed in the shell of nucleus accumbens and MPA in both mouse and monkey. In other cases, such as a few midline thalamus/hypothalamus primary SCN target areas including PVT, CM, and PVN as well as in Re, a midline thalamic nucleic, PK2 is strongly expressed in the monkey brain but not in the rodent. (Note that in general, the PKR2 mRNA signal is higher in mouse vs. monkey brain tissue. Therefore, areas positive for PKR2 expression in monkey but not in mouse, such as raphe n., are likely to represent a species difference.)

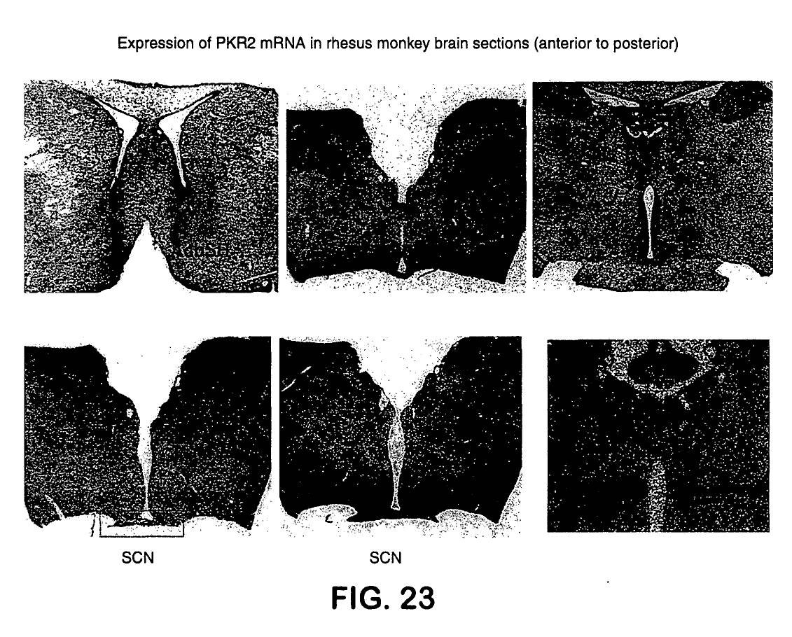

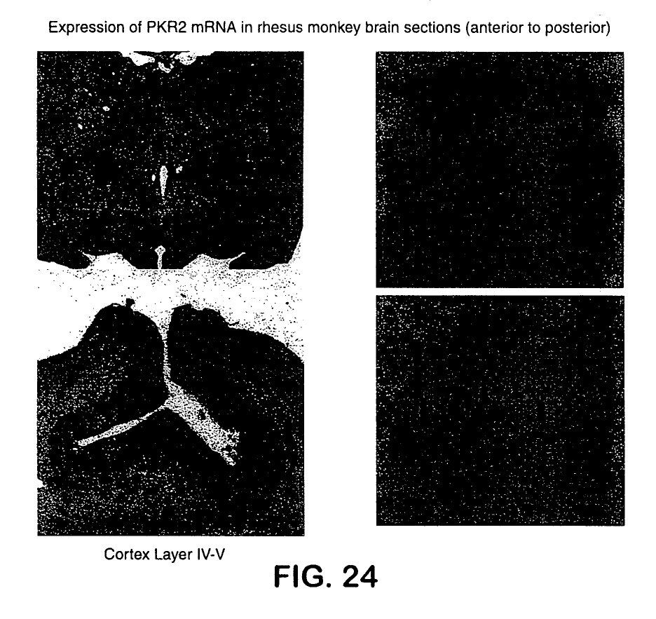

PKR2: PKR2 is expressed in SCN target nuclei (such as PVT, PVN, LS, DMH, and BST) in rhesus monkey brain (FIGS. 23 and 24): similar to what is observed in mice. In general, however, the mRNA levels are lower in monkey vs. mouse nuclei. PKR2 is also expressed in the SCN itself in monkey brain (FIG. 23) as has been observed in mouse brain tissue.

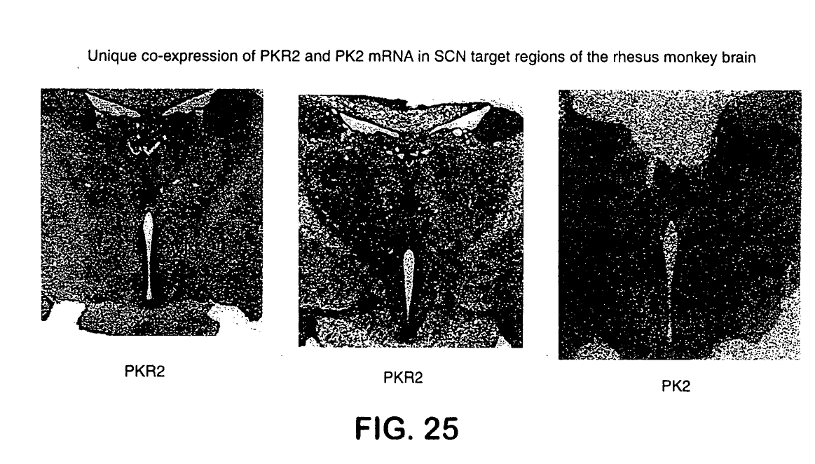

Potentially important differences in the expression pattern of PKR2 in monkey vs. mouse brain tissue are observed, however: PKR2 is strongly expressed in the dorsal raphe N. (reticular activation system) and SC (visual input and output area) in the monkey but not in the mouse. This finding suggests that PKR2 in the SC may be a downstream target of the SCN in diurnal animals, and activation of PKR2-positive neurons in these primary targets may modulate the sensory/motor functions in the monkey. In contrast to the high level of expression of PKR2 at the mRNA level in mouse area postrema (AP), PKR2 is not detected in the AP of monkey brain. The AP is one of three brain circumventricular organs that sense the chemical environment of the systemic circulation. AP is not known to be involved in regulation of circadian locomotor, or sleep/wake rhythm. Co-expression of PKR2 and PK2 mRNA in SCN target regions is observed in the rhesus monkey brain but not in the mouse brain (FIG. 25).

The PKR2 in situ experiments were mainly carried out with sections derived from animal 20528 (sacrificed at 3 am). The PK2 image is from animal number 15452 sacrificed at approximately 9 am. Earlier, we carried out PKR2 in situ experiment with sections from animal (ID15452) (sacrificed at approximately 9 am) with similar results.

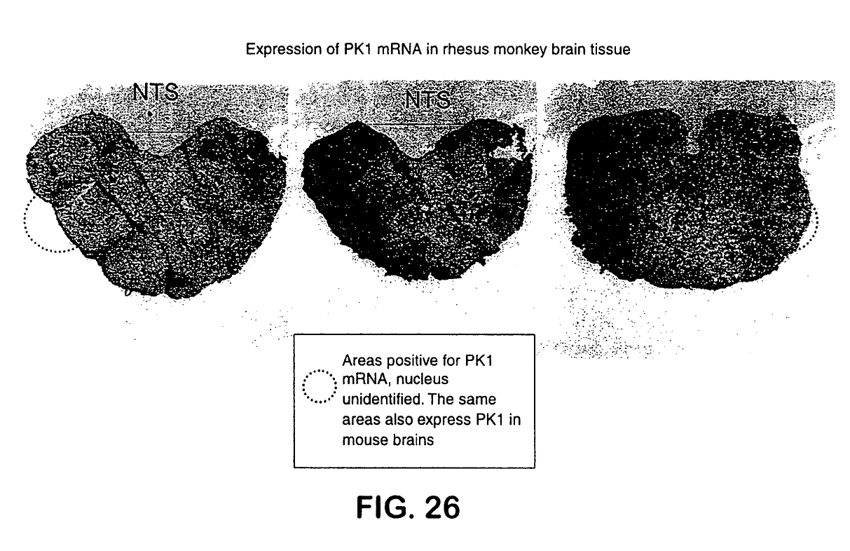

PK1: PK1 mRNA expression is only detected in specific midbrain and brainstem areas in the monkey brain (FIG. 26).

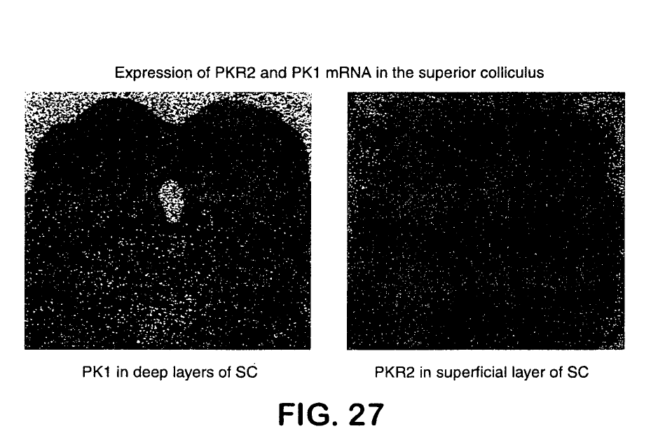

The positive areas include NTS (an area also positive for PK1 in mouse brain) and the deep layer of the SC (negative for PK1 expression in mouse brain tissue). As described earlier, the SC also expresses PKR2 (FIG. 24). The deeper layers of the SC are thought to send excitatory descending motor commands to the brainstem and spinal cord. It is interesting to note that PK1 and PKR2 are expressed in different layers of the SC: PK1 is expressed in the deep layer of the SC whereas PKR2 is expressed in the superior layer. Whether there are any synaptic connections between these PK1-expressing and PKR2-expressing layers is not known, but there appears to be an intra-columnar projection from the superficial layers (PKR2-positive) to deep layers (PK1-positive) (FIG. 27).

The PK1 in situ experiments were carried out with sections derived from animal ID number 21670 (sacrificed at 11 pm).

PKR1: PKR1 (prokineticin 1 receptor) is not detected in any of the monkey brains sections examined, including blocks of cortex, thalamus/hypothalamus and brainstem. In situ studies performed with mouse brain tissue revealed a low signal for PKR1 in the NTS as well as the CA1/CA3 of the hippocampus (see appendix). In contrast, PKR1 mRNA is not detectable in these corresponding areas of the monkey brain (not shown).

In situ experiments with the PKR1 probe were carried out with sections derived from animal ID number 20528 (sacrificed at 3 am). Positive control studies are ongoing with monkey spinal cord and dorsal root ganglion and testes tissue is an alternative control tissue source.

Discussion: A primary objective of this study was to demonstrate the presence of PK2 and determine its oscillation phase in the SCN of the rhesus monkey brain. This study confirms that PK2 mRNA is present in the monkey SCN and that it oscillates in a high amplitude manner. The oscillation phase for PK2 in the monkey (diurnal) SCN is the same as that observed in the nocturnal mouse; that is, there is a high level of PK2 expression during the day phase and low level of expression during the night phase. The oscillation of melatonin levels in the blood taken from the same monkeys is in the opposite phase to that observed for PK2.

Based on utilizing monkey specific PK and PKR probes, the distribution patterns of PK2, PKR2, PK1 and PKR1 in rhesus monkey brain have been carefully examined. There are similarities but also some potentially important differences in the expression of PK2 and PKR2 in monkey vs. mouse brain tissues. For example, PKR2 is strongly expressed in the dorsal raphe N. (reticular activation system) and superior colliculus (visual input and output) in the monkey but not the mouse brain. Thus, activation of PKR2-positive neurons may modulate sensory/motor functions in the monkey. Preliminary results also suggest that PKR2 is expressed in layer 4-5 of cortex of monkey brain (only sporadic expression of PKR2 is observed in cortex of mouse brain). Efforts to determine whether these cortical regions correspond to the sensory or motor cortices or other cortical regions in the monkey are ongoing.

In addition to the SCN, PK2 is also expressed in primary SCN targets (PKR2-positive) in monkey brain (FIG. 25), but not in the corresponding areas in the mouse brain. In particular, PK2 is highly expressed in a few midline thalamus/hypothalamus nuclei that also express PKR2, including PVT, CM, and PVN. Midline hypothalamic/thalamic nucleic are thought to be crucial for arousal and are likely the most important SCN output targets (Moore et al., Annu Rev Med. 48:253-66, (1997); Van der Werf et al., Brain Res Brain Res Rev. 39:107-40 (2002)). Recently, it has been shown that PK2 stimulates the firing of PKR2-positive neurons (Cottrell et al., J Neurosci 24:2375-9 (2004)). This excitatory effect of PK2 on PKR2-neurons suggests that there is a positive feedback mechanism operating in these primary SCN targets. This positive feedback mechanism may explain why the SCN circadian clock has been thought to control both the duration and timing of sleep/wake cycle in diurnal monkeys, but only the timing and not the duration of sleep/wake cycle in nocturnal animals. [SCN-lesions in monkeys lead to a loss of timing of sleep/wake cycle and also prolong sleep duration (Edgar et al. J. Neurosci. 13:65-79 (1993)). In contrast, SCN lesions in rodents appear to only affect the timing of sleep/wake cycle but not the duration (Mouret et al. Electroencephalogr Clin Neurophysiol. 45:402-408 (1978)); Eastman et al. Physiol Behav. 32:357-368 (1984); Mistlberger R E, et al. Sleep JID 10:12-24 (1987)).

In summary, this study confirms the presence and oscillation of PK2 mRNA in the suprachiasmatic nucleus (SCN) of diurnal primates and the expression of PKR2 in the SCN target nuclei. Thus, the validation of PKR2 as a drug target for circadian rhythm disorders, especially sleep/wake disorders, is supported by the in situ hybridization studies in the rhesus monkey brain. Potentially important differences in the expression patterns of PK2 or PKR2 mRNAs are observed in some areas of the brain in diurnal vs. nocturnal animals. These differences, as well as the same phase of PK2 oscillation in both nocturnal and diurnal animals, highlight the importance of conducting studies in the monkey or other diurnal species to support drug discovery and development efforts.

Abbreviations: AcbSH, nucleus accumbens shell; AP, area postrema; BST, bed nucleus of stria terminalis; MnPO, medial preoptic area; CM, centromedial nucleus of thalamus; DMH, dorsomedial nucleus of hypothalamus; DR, dorsal raphe nucleus; LS, lateral septum; NTS, nucleus tract solitarius; PVT, paraventricular nucleus of thalamus; PVN, paraventricular nucleus of hypothalamus; Re: Reuniens thalamus nucleus; SC, superior colliculus; SCN, suprachiasmatic nucleus

### EXAMPLE XIII

### Validation of PKR2 as a Drug Target for Circadian Rhythm Disorders: Effect of PK2 on Rest-Activity Rhythm and Eating Behavior in Normal Rats

Summary: Specific physiological and behavioral processes, including sleep/wakefulness, feeding, and body temperature, exhibit circadian rhythm. In this study, the effect of PK2 on two circadian behaviors, i.e., wheel-running activity and food intake, was studied in normal rats. These findings show that PK2 inhibits wheel-running activity in a dose-dependent manner consistent with the effect of PK2 on sleep-related parameters.

This study also shows that acute administration of PK2 at either daytime or nighttime inhibits food intake. Feeding behavior is strongly regulated by the circadian clock; about 80% of total food intake by rats occurs during the dark period.

These findings support the key role of PKR2 in the regulation of circadian-controlled behavioral responses and as a drug target for circadian rhythm disorders.

Background: Circadian rhythms have been observed for a range of physiological and behavioral processes, such as sleep/wakefulness, feeding, and body temperature. Of these, the regulation of the circadian sleep/wake cycle by the suprachiasmatic nucleus (SCN) is most obvious and crucial. In rodents, activity patterns correlate well with stages of wakefulness and sleep; approximately 95% of wheel-running activity occurs in the night phase. Wheel-running activity, which has a high signal-to-noise ratio, provides an accurate way of assessing sleep/wakefulness rhythm.

The inhibitory effect of acute delivery of PK2 into the lateral ventricle of the brain on wheel-running behavior has been previously demonstrated. In this study, the effect of delivering PK2 into the 3^{rd} ventricle was examined. While the precise locations of the SCN target(s) mediating the control of circadian locomotor rhythm are not entirely known, it is generally agreed that the target areas lie close to the SCN. Thus, delivery of PK2 via infusion into the 3^{rd} ventricle may better mimic the effect of the PK2 signal output from the SCN. The 3^{rd} ventricle was also selected as the optimal site of delivery of PK2 for the EEG studies, and it is desirable to correlate locomotor and sleep activities under similar experimental conditions.

The diverse rhythms under the control of the SCN clock have a specific temporal relationship with each other and are synchronized with the environmental light-dark cycle. It seems unlikely that the SCN clock sends different, unique timing signals (controlled by a transcriptional/translational loop) to regulate specific physiological and behavioral rhythms. Rather, it is more plausible that the SCN clock sends one or a very limited number of circadian loop-controlled timing signals that ultimately drive different physiological/behavioral rhythms.

Based on our recent molecular, pharmacologic and genetic studies, we have shown that PK2 is a key regulator for circadian rest-activity rhythm. The high expression of PKR2 in the PVN, a pivotal SCN target for the autonomic and endocrine functions, suggests that PK2 may also regulate food intake. This hypothesis is further supported by our recent functional studies showing that PK2 activates the electrophysiological activity of the PVN (Yuill, Erik A., Catharine C. Ferri, Qun-Yong Zhou and Alastair V. Ferguson Prokineticin 2: A Circadian Regulator of Neuroendocrine and Autonomic Function. Manuscript (attached)).

Methods: Male adult rats (Charles River Laboratories, age 90 days) were cannulated under anesthesia ketamine and xylazine. A stainless steel cannula (Plastics One, Wallingford, Conn.) was stereotaxically implanted within the lateral ventricle (0.4 mm rostral to bregma, 3.0 mm ventral to dura, 2.4 mm lateral to midline) or third ventricle (AP, 1.8 mm caudal to bregma, H, 4.5 mm ventral to bregma, Lat, 0 mm). A 30-gauge stylet was placed in the guide cannula to maintain patency. Rats were allowed to recover for one week and then housed in running wheel cages and entrained to 12:12 LD cycle for >2 weeks. Rats were given recombinant PK2 at either ZT14 or ZT2. During the injection, the stylet was removed and a 30-gauge injector attached to a Hamilton syringe by plastic tubing was inserted. Each animal received either 5 μl of recombinant human recombinant PK2 (either as 0.1 or 1 μg/ul for low or higher dose, as indicated) or saline over a 2-minute period. Animals were immediately returned to their cages and wheel-running activities were monitored with a computer system (Colbourn Instrument, Allentown, Pa.). Running-wheel data were analyzed at intervals of 5 minutes with an in-house software program. Wheel-running activity was expressed as a percentage of average counts obtained from the corresponding time interval two days prior to PK2 (or saline) administration. The wheel-running data collected in the first hour immediately after PK2 or saline injections were excluded due to the interference from the handling effect.

For the food intake experiments, rats were housed in regular cages with free access to food and water, and food intake was measured approximately every 4 hrs during the first 24 hr after PK2 or saline delivery by weighing the food.

All data were plotted as the mean±SEM, and t-test was used for all statistical analysis.

Effect of PK2 (3^{rd} ventricle) on wheel-running activity: Previously, we showed that the administration of PK2 (5 μg) at ZT14 via the lateral ventricle strongly inhibits the nocturnal wheel-running activity. In this study, we tested the effect of delivery of PK2 via infusion into the 3^{rd} ventricle since a) this delivery site may better mimic the effect of the PK2 signal output from the SCN and b) it is desirable to have similar experimental conditions to correlate with the sleep/wake study.

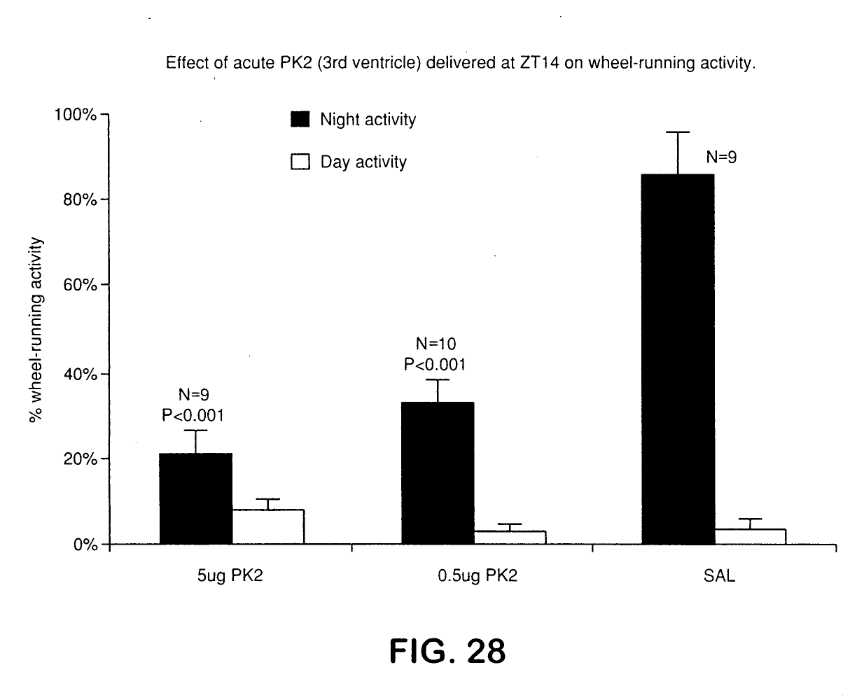

Administration of PK2 (5 μg) into the 3^{rd} ventricle strongly inhibits nocturnal wheel-running activity (FIG. 1A). This effect is similar to what was observed upon injection into the lateral ventricle. Administration of the ten-fold lower dose of PK2 (0.5 μg) into the 3^{rd} ventricle also significantly inhibits nocturnal wheel-running activity (FIG. 28).

The effect of PK2 on night activity is dose-dependent. The maximum wheel-running inhibition is about 80% for the higher dose PK2 (5 μg) vs. 65% for the lower dose of PK2 (0.5 μg). The higher dose of PK2 slightly stimulates the activity during the day time hours, likely due to a rebound effect; a rebound effect was not observed with the lower PK2 dose.

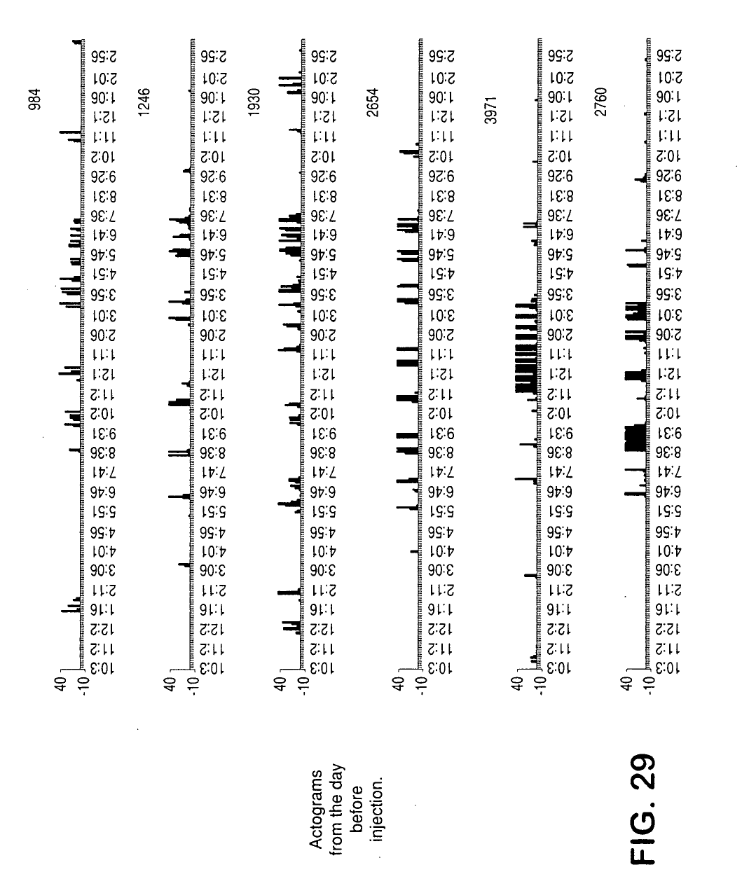

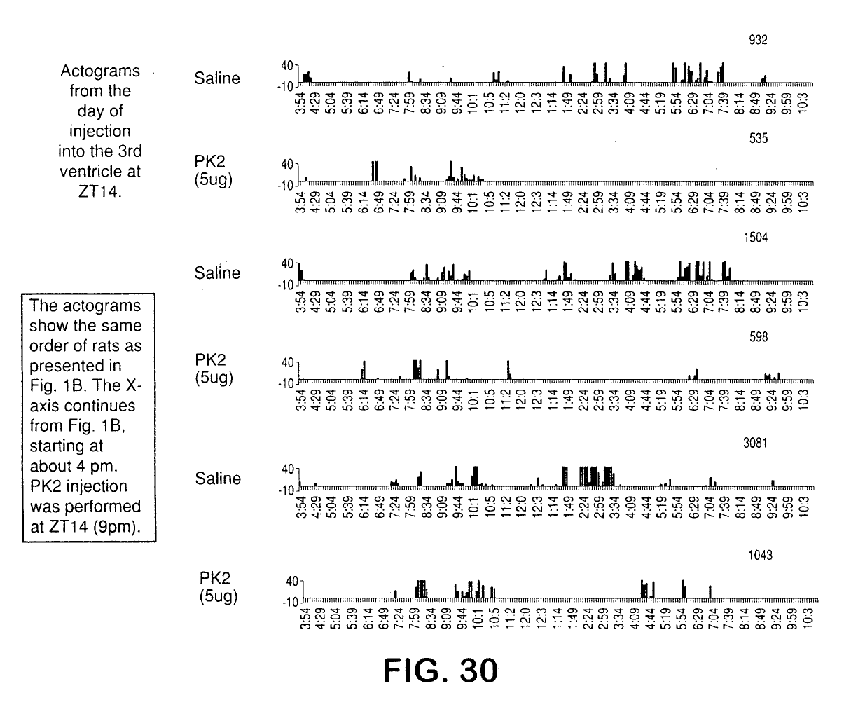

Representative actograms from six rats (3 treated with PK2 and 3 treated with saline) are shown in FIG. 29 (the day prior to injection) and FIG. 30 (the day of injection). Upon visual inspection, it is clear that PK2 treatment strongly inhibits the nocturnal wheel-running activity, while saline has a negligible or no effect.

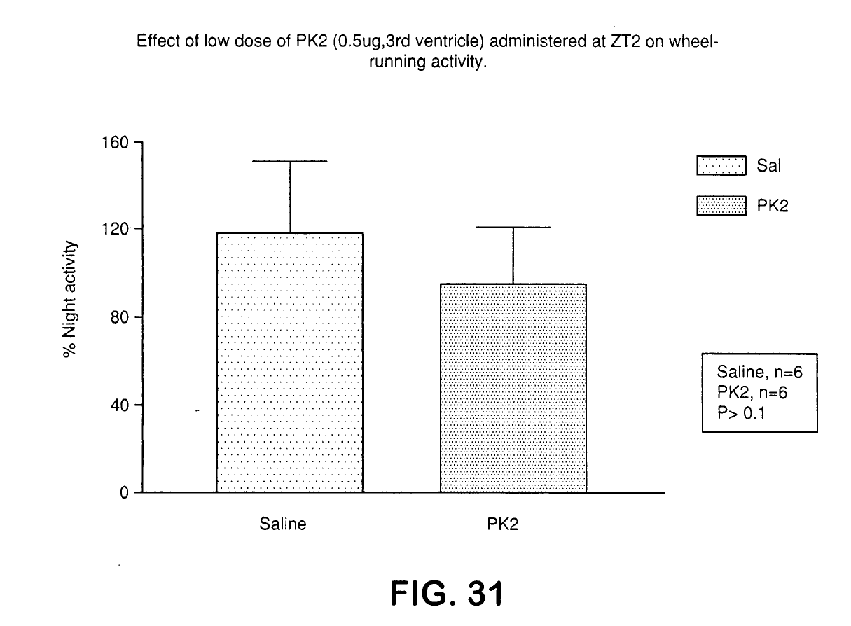

Endogenous PK2 levels are high during daytime and low during nighttime. To examine whether there is a light/dark phase-dependent effect of PK2 on circadian behavior, PK2 (0.5 μg) was delivered into rats via the 3^{rd} ventricle at daytime (ZT2). FIG. 31 shows that the low dose of PK2 at ZT 2 has no significant effect on nighttime wheel-running activity.

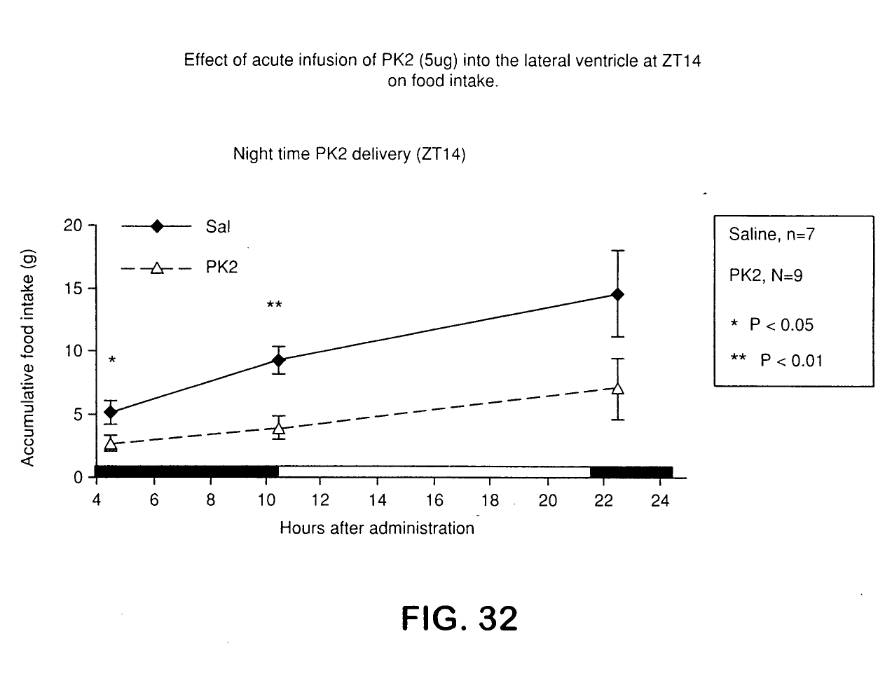

PK2 (lateral ventricle) on food intake and correlation with wheel running activity: The effect of PK2 on food intake was examined. FIG. 32 shows that PK2 delivered at ZT14 significantly inhibits food intake. The inhibition is greatly than 70% over the entire nighttime period. This ° of inhibition is similar to that reported for the melanocortin agonists, one of the most potent appetite inhibitors (Fan et al., Nature. 9;385(6612):165-8 (1997)).

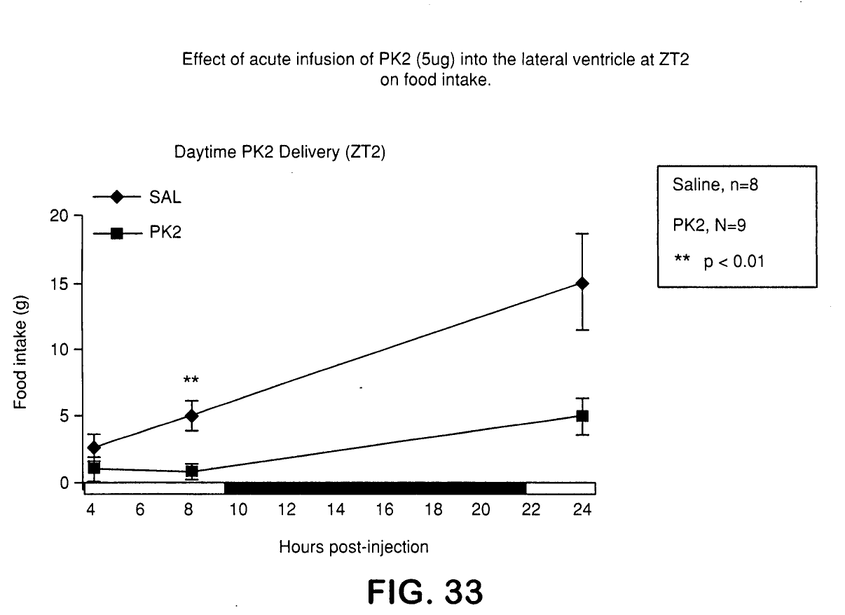

To examine whether there is a light/dark phase-dependent effect of PK2 on food intake behavior, PK2 was delivered at daytime (ZT2) when endogenous PK2 levels are high. FIG. 33 shows that PK2 also inhibits food intake, upon delivery at daytime and that the degree of inhibition is prominent and lasts for 8 hours or more.

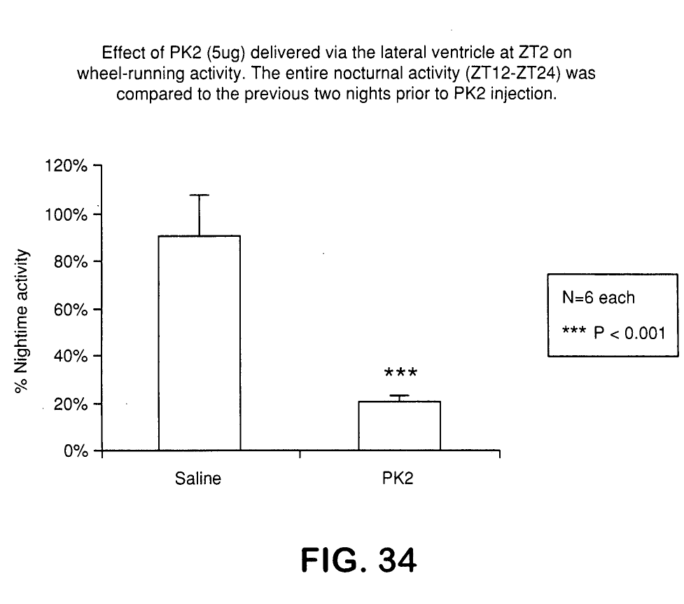

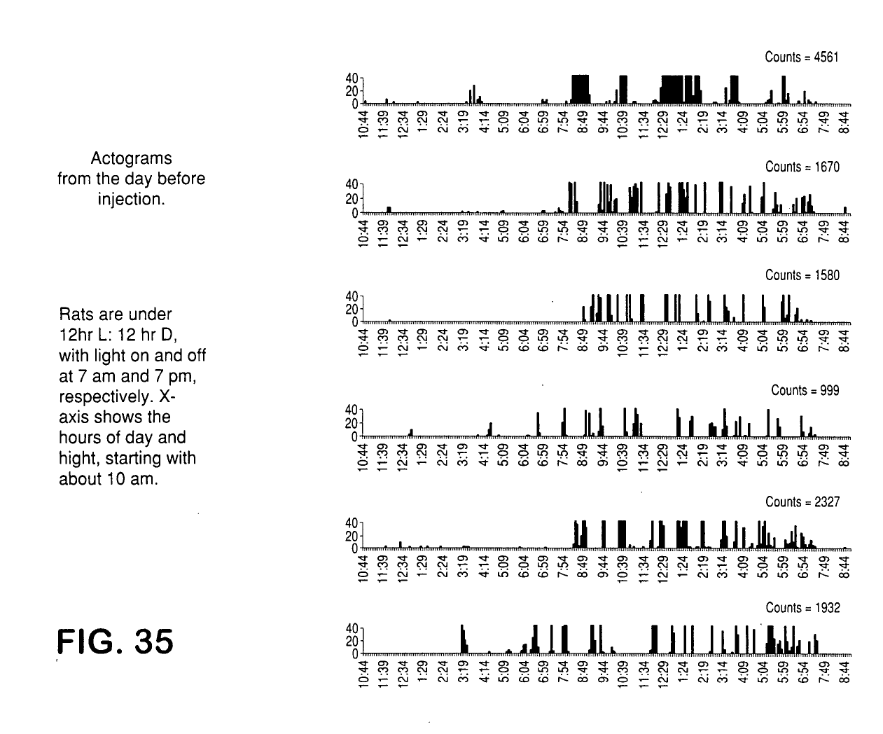

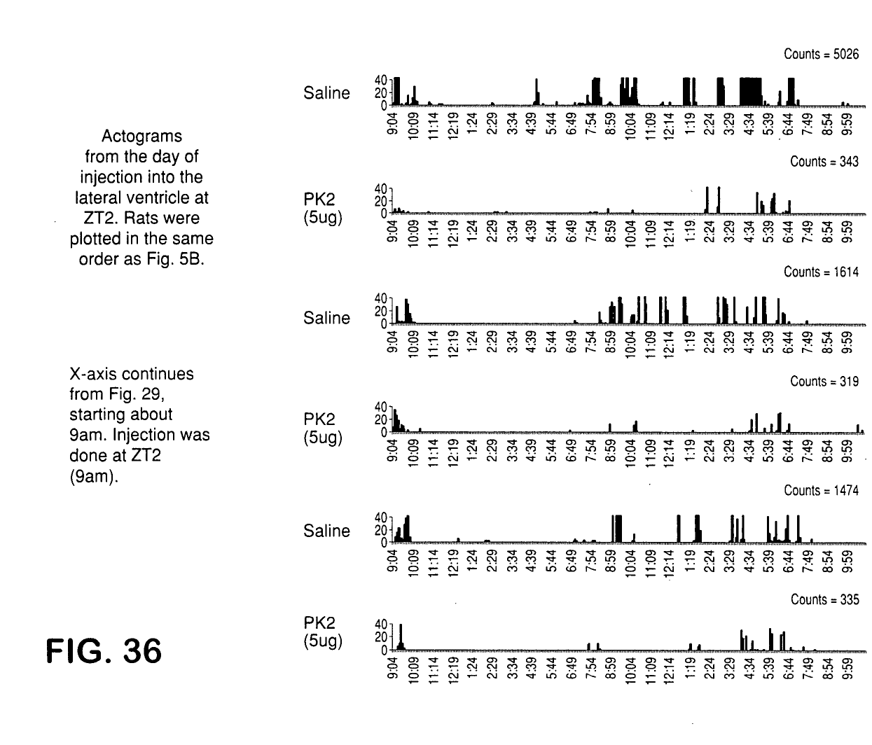

The inhibitory effect of PK2 on food intake when delivered via the lateral ventricle at daytime (ZT=2) prompted us to examine further the effect of the same dose of PK2 on wheel-running activity via the same route of delivery. FIG. 34 shows that administration of PK2 via the lateral ventricle at ZT2 inhibits nocturnal activity, particularly during the early part of the night (FIGS. 35 and 36).

Conclusion/Discussion: The foregoing pharmacological studies confirm that PK2 is a key regulator of circadian activity. Wheel-running activity is modulated by PK2 in a dose- and phase-dependent manner. The higher dose of PK2 (5 μg) has a more robust activity in terms of inhibiting nocturnal wheel-running activity than the lower PK2 dose (0.5 μg). Also, while the lower dose of PK2 (0.5 μg) is effective in inhibiting wheel-running activity when administered at ZT14, it has essentially no effect when administered at ZT2.

These activity studies are in agreement with EEG recording, showing that PK2 modifies parameters of sleep/wakefulness stages. For example, PK2 strongly inhibits REM sleep when administered at ZT2 or ZT14. REM sleep may be viewed as a period of central motor activation, and therefore, the suppression of REM sleep by PK2 and the suppression of wheel-running activity by PK2 may be closely related phenomena.

This study also shows that PK2 strongly inhibits food intake. Whether the PK2 inhibition of food intake is secondary to its activity suppression effect is currently unclear. However, appetite is known to be under the control of the SCN clock and some appetite-related regulators oscillate in a SCN-dependent manner (Kalra et al., Endocr Rev. 20(1):68-100 (1999)).

Based on a number of observations, we propose that the effect of PK2 on the PVN may be especially relevant to food intake. The PVN is known to be a central regulator of food intake behavior. We have shown that there is a high level of expression of PKR2 in the PVN in both rodents and monkeys. In collaboration, we have also recently found that PK2 activates the electrophysiological activity of the PVN (Yuill et al., Prokineticin 2: A Circadian Regulator of Neuroendocrine and Autonomic Function. Manuscript (attached)). The possible regulation of multiple circadian rhythms by PK2 suggests that targeting the PK2 signal from the SCN clock may have the potential to influence chronic metabolic diseases such as diabetes, hypertension, and depression. There is also growing evidence that it may be possible to detect circadian disturbances and disorders before the development of certain disease states in humans (Buijs et al., J Endocrinol. 177:17-26 (2003)).

The SCN circadian clock regulates a range of physiological and behavioral processes. These diverse rhythms have a specific temporal relationship with each other and are synchronized with the environmental light-dark cycle. It seems unlikely that the SCN clock sends a unique timing signal (controlled by transcriptional/translational loop) to regulate specific physiological/behavioral rhythms. Rather, it is more plausible that the SCN clock sends one or a very limited number of circadian loop-controlled timing signals that ultimately drive different physiological/behavioral rhythms. Based on molecular, pharmacologic and recent genetic studies, we have demonstrated that PK2 is a key regulator of circadian controlled behaviors.

### EXAMPLE XIV

Validation of PKR2 as a Drug Target for Sleep-Wake Disorder

The objective of this study is to validate PKR2 as a drug target for sleep-wake disorder. Previous studies showed an effect on the circadian rhythm of behavior, i.e., wheel-running activity, and it was important to determine the direct effect of PK2 on sleep related parameters. To achieve this objective, PK2 was administered acutely to rats at night (ZT14) and in the daytime (ZT2), and the subsequent effects on sleep parameters were determined.

This study shows that PK2 strongly suppresses REM sleep for 10 hours, with the most marked suppression within the first 5 hours following PK2 administration. This effect is observed with injections at either ZT2 or ZT14. REM sleep can be viewed as a period of central motor activation; therefore, this effect on REM sleep correlates with the suppression of motor activity by PK2.

The effect of PK2 injection on NREM sleep time is smaller in comparison with the effect on REM sleep time. When given at ZT14, during the dark period when sleep amounts are minimal in the rat, there was an initial slight suppression of NREM sleep followed by an increase. When given at ZT2, during the light period when sleep time is maximal, there is a non-significant suppression of NREM sleep during the second half of the light period, as sleep drive declines. The net effect of PK2 administration at both ZT2 and ZT14 on the sleep-wake distribution is a flattening of the circadian distribution of sleep.

These findings demonstrate the key role of PKR2 in sleep regulation and support PKR2 as a drug target for sleep disorders. It is also interesting to note that several drugs affecting the release or metabolism of monoamines produce REM sleep suppression of similar magnitude and have potent anti-depressant effects.

Sleep regulation is modeled as a two-process system consisting of a homeostatic process and a circadian timing process, which together determine sleep propensity and the length, intensity and incidence of sleep episodes (Borbely et al., Hum Neurobiol JID 8211530 1, 195-204 (1982)). It is clear that there is circadian control of the sleep-wake cycle, as this cycle continues to occur with a period of nearly 24 hours in the absence of environmental time cues (Czeisler et al., Science JID-0404511 210, 1264-1267 (1980)). Early studies of lesions of the suprachiasmatic nucleus (SCN) of the hypothalamus in rats provided strong evidence that the SCN controls the normal circadian expression of the sleep-wake cycle, but also showed that the total daily amount of sleep was not affected by loss of the SCN (Eastman et al., Physiol. Behav. 32:357-368 (1984); Mistlberger et al., Sleep 10:12-24 (1987); Mouret et al., Electroencephalogr Clin. Neurophysiol. 45:402-408 (1978)). Thus, the circadian process, governed by the SCN, of timing the occurrence of sleep was thought to be independent of the homeostatic process of sleep regulation; the hypothesis that these processes are independent was also supported by the finding that recovery sleep amounts after sleep deprivation were unchanged in SCN-lesioned rats (Mistlberger et al., Sleep 10:12-24 (1987); (Mistlberger et al., Sleep JID-7809084 6, 217-233 (1983); Tobler et al. Neurosci Lett JID-7600130 42:49-54 (1983)). However, results of more recent SCN lesion studies in squirrel monkeys have indicated that there is an interaction between these circadian and homeostatic processes, as complete lesions of the monkey SCN produced both a loss of the circadian timing of sleep and a 4 hr increase in daily sleep time (Edgar et al., J. Neurosci. JID-8102140 13:1065-1079 (1993)). This finding led to Edgar's “opponent process” model, which posits that the SCN clock produces a “wakefulness-promoting” signal that facilitates arousal and thereby actively opposes the homeostatic sleep drive (Edgar et al., J. Neurosci. JID-8102140 13:1065-1079 (1993)). Results from clk/clk mutant mice appear to be consistent with the hypothesis that the circadian clock, in addition to the sleep homeostat, plays an important role in determining total sleep time. Importantly, clk/clk mutant mice sleep 2 hours less per day than do wild type mice, indicating that the absence of circadian signaling in these mutant mice may suppress total sleep time (Naylor et al., J. Neurosci. JID-8102140 20:8138-8143 (2000)). Thus, a circadian clock-generated signal may promote sleep in nocturnal mice. Taken together, these findings suggest that the circadian system, seated in the SCN, may be involved in regulating total sleep time in addition to the timing of sleep.

Q-Y Zhou and co-workers discovered a protein, prokineticin-2 (PK2) (Li et al., Mol Pharmacol JID-0035623 59:692-698 (2001)), that is synthesized in discrete regions in the brain; in particular, PK2 is expressed in the SCN, and its expression is governed by the molecular components of the circadian clock (Cheng et al., Nature JID-0410462 417: 405-410 (2002)). We also recently observed that acute infusion of PK2 inhibited nocturnal wheel-running behavior (Cheng et al., Nature JID-0410462 417: 405-410 (2002)). This finding was consistent with two hypotheses. One hypothesis was that activity levels would correlate highly and negatively with sleep amounts, and that PK2 infusion would increase either total sleep percentage, nonREM sleep percentage or REM sleep percentage; the other possibility was that PK2 would have an effect on activity that is independent of any effect on sleep timing or duration. To test these hypotheses, we have examined the effects of acute infusions of PK2 on rapid eye movement (REM) and non-REM (NREM) sleep times.

Animal preparations for sleep/wakefulness experiments: Male rats (Sprague-Dawley, 300-350 g, Charles River Laboratory) were surgically implanted with EEG and EMG recording electrodes and a guide cannula into the third ventricle (3V). Anesthesia was induced by ketamine/xylazine injection (90/13 mg/kg body weight, i.p.) and maintained with isoflurane inhalation (0.5-2.0%). Animals were placed in a stereotaxic apparatus (David Kopf Instruments, Tujunga, Calif.), and 4 stainless steel screw electrodes were inserted stereotaxically in the skull at the following coordinates: for maximizing recording of theta activity, AP, 3 mm rostral to bregma, Lat, 1 mm, and AP, 5 mm caudal to bregma, Lat, 3 mm; pair for minimizing theta, AP, 1 mm caudal to bregma, Lat, 3 mm, and AP, 5 mm caudal to bregma, Lat, 4 mm. Flexible stainless steel wire electrodes were implanted into dorsal cervical musculature for EMG recording. A 23 gauge guide cannula together with a 30 gauge obturator (CMA/11, CMA/Microdialysis AB, North Chelmsford, Mass.) was implanted into 3V at the following coordinates: AP, 1.8 mm caudal to bregma, H, 4.5 mm ventral to bregma, Lat, 0 mm. The implants and an attached head plug, which serves as an interface with the recording equipment, were attached to the skull with cyanoacrylate and dental cement. One week to 10 days after surgery, recording cables were attached to rats' head plugs and the rats were placed in the recording chamber. Rats were acclimated to the recording setup for 1 week before data collection began. Rats were housed with 12 hr light/dark (LD) cycles.

Acute PK2 and aCSF infusions: For examining the acute effect of PK2 on vigilance, recombinant PK2 was administered at ZT14, when endogenous PK2 levels are low. To compare the time-dependent effect, a separate group of rats was injected at ZT2, when endogenous PK2 is high. During ICV injections, the obturator was removed and a 29 gauge cannula, attached to a Hamilton syringe by plastic tubing, was inserted. On day 1 of the injection protocol, angiotensin II was injected to verify correct cannula placement in 3V. 500 ng of angiotensin II (Peninsula Laboratories, Inc., San Carlos, Calif.) was injected in 3 μl over 1 min., followed by a 30 sec waiting period, after which animals were returned to their cages; animals with correct placement of the injection cannula started drinking within 1-2 min after being returned to their cages and showed a robust drinking response. A baseline recording was made on day 3, and either PK2 or artificial cerebrospinal fluid (aCSF) was injected on day 4 at either ZT2 or ZT14. aCSF or 0.5 μg PK2 was injected in 5 μl; aCSF or 5 μg PK2 in 8 μl was administered to 5 pairs of rats at ZT14 and to 5 pairs of rats at ZT2.

Data collection of sleep/wakefulness: EEG and EMG activities were recorded continuously on a 18 channel GRASS 78 polygraph (Grass Telefactor, West Warwick, R1) using Spike2 software (Cambridge Electronic Design, Cambridge, UK). Polygraphic recording was carried out as described (Wu et al., 1999; John et al., 2000). The system monitored amplified (×10,000) EEG (bandpass, 1-30 Hz; digitization rate, 100 Hz) and EMG (bandpass, 5-30 Hz). Vigilance states were visually classified as NREM sleep, REM sleep, active wake and quiet wake in 30 sec epochs according to previously established criteria (Bergmann et al. Sleep JID-7809084 10:1-11 (1987); Neckelmann et al., Sleep JID-7809084 16:467-477 (1993)). Briefly, wakefulness was identified by the presence of desynchronized EEG and phasic EMG activity. Active waking was scored when EMG amplitude reached 15 times the average NREM sleep EMG amplitude over >50% of the epoch, reflecting a substantial amount of movement; waking epochs with lower EMG amplitude and more stable EMG voltages were scored as quiet waking. NREM sleep consisted of high amplitude slow wave EEG together with low EMG tone relative to waking. REM sleep was identified by the presence of regular theta EEG activity together with absent EMG tone. Low voltage sleep, as defined by Bergmann et al., and the transition state between NREM and REM sleep were scored as NREM sleep. Data from all time points were included in the analysis.

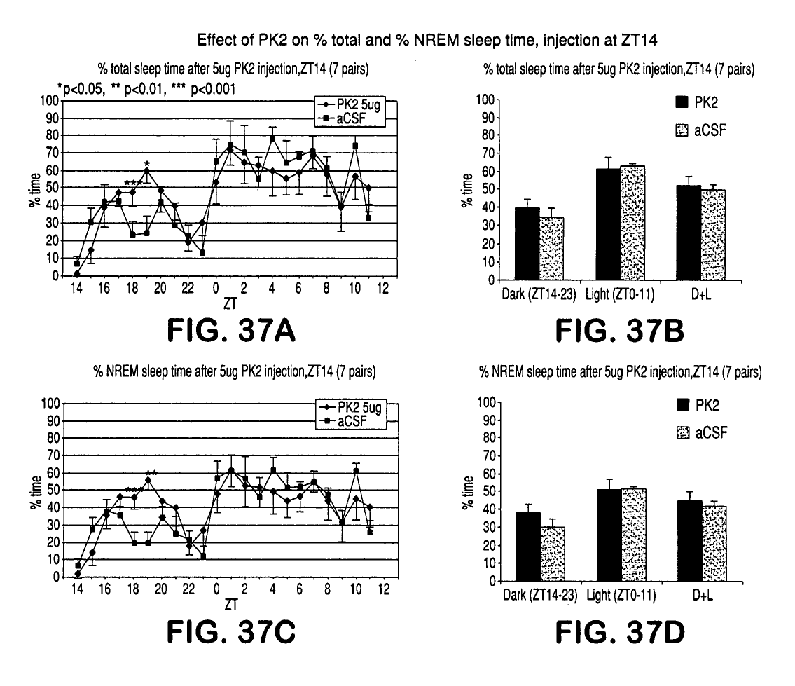

Effect of acute infusions of PK2 at ZT14 on vigilance levels: Seven pairs of infusions of either 5 μg PK2 or aCSF (controls) were administered at ZT14. There was a significant increase in % total sleep time at ZT18 and ZT19 following PK2 administration (p=0.001 and p=0.018, respectively; t-test); however, there was no significant increase in % total sleep time in the dark period (ZT 14-23) following infusion, and no change in % total sleep time in the following light period (FIG. 37A, B).

In parallel with the effect on % total sleep time, there was a significant increase in % NREM sleep time at ZT18 and ZT19 following PK2 administration (p=0.000 and p=0.104, respectively; t-test), followed by a non-significant increase over the remainder of the dark period (FIG. 37C, D). Over the first 8 hours following injection, % NREM sleep time was increased (p<0.05, t-test). Over the 10 hours of the night period following injection, % NREM sleep time was non-significantly increased; over the following light period, % NREM sleep time remained largely unchanged.

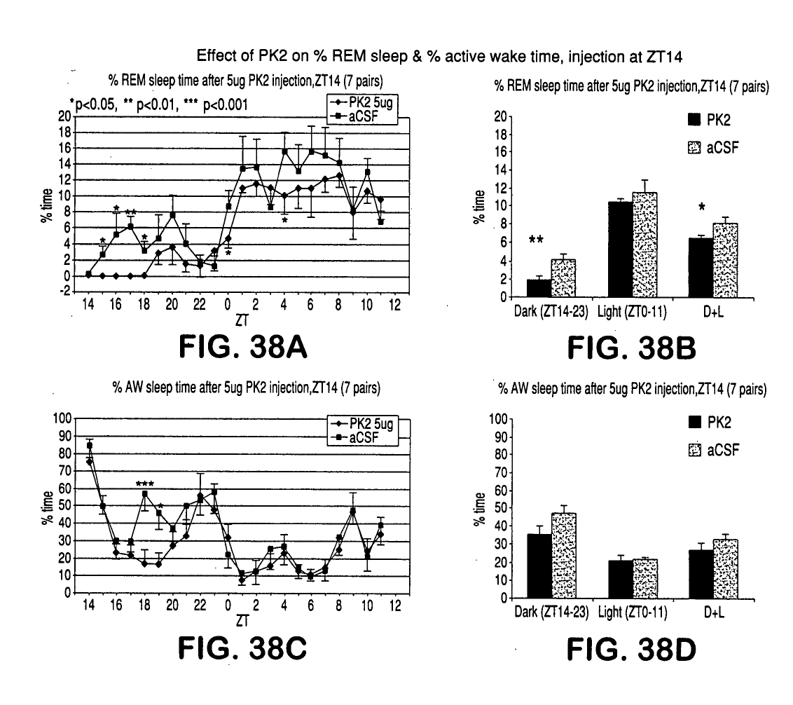

Percent active wake time was significantly decreased at ZT18 and ZT19 in the dark period following PK2 administration at ZT14 (p=0.001 and p=0.018, respectively; t-test), but was not significantly changed in the following light period (FIG. 38C, D).

PK2 infusion strongly suppressed REM sleep for 9 hours following injection, to 40% of control levels for that period (p=0.010, t-test); there was almost no REM sleep for the remaining hour of the dark period after injection (FIG. 38A, B). No rebound in REM sleep was observed in the following dark period. Overall, % REM sleep time was reduced over the 22 hours following injection at ZT14, compared to control animals (p=0.027, t-test).

Note: p values are determined by Student's t test.

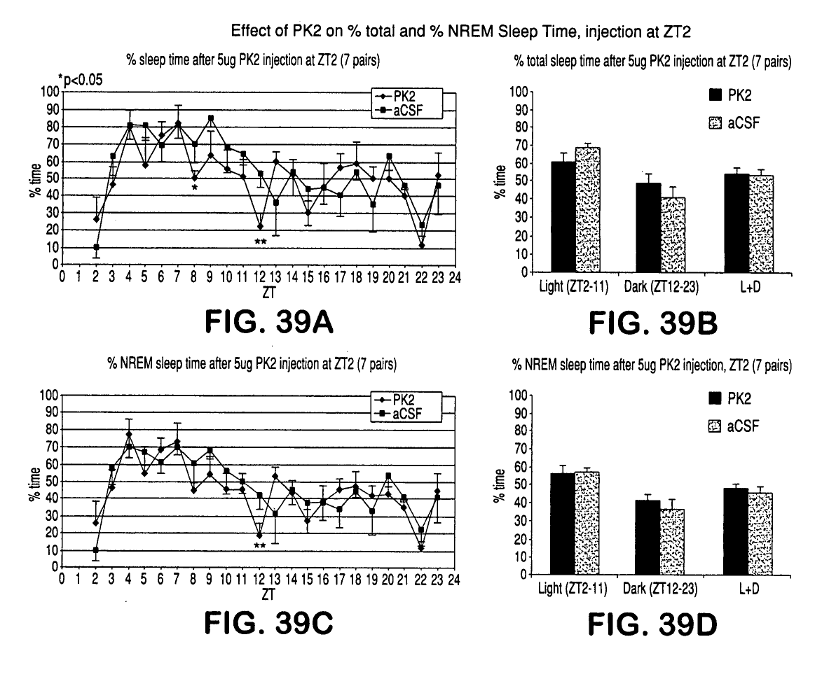

Effect of acute infusions of PK2 at ZT2 on vigilance levels: A total of seven pairs of injections of either 5 μg PK2 or aCSF (control) have been administered. With injection at ZT2, during the light period when sleep is maximal, there is a minimal effect on NREM sleep during the light and dark periods (p=0.350, 0.346, respectively) compared to controls (FIG. 39C, D). These changes contribute to a non-significant effect on total sleep time in the light and dark periods (p=0.090, 0.286, respectively) following PK2 infusion (FIG. 39A, B).

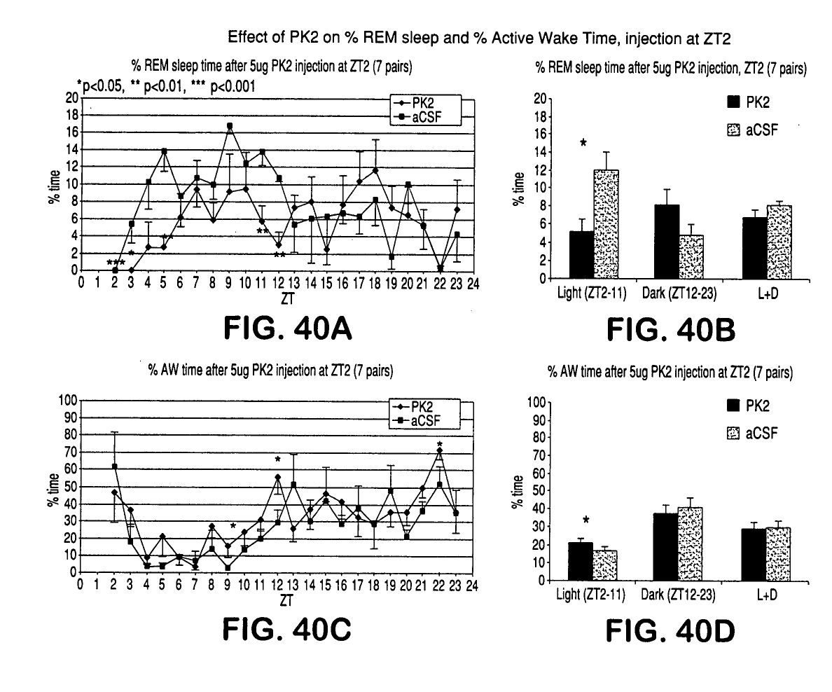

There is small but a significant increase in active wake time during the light period (p=0.049) following injection, in parallel with the corresponding non-significant decrease in sleep time; however, no change in active wake time is observed in the following dark period (FIG. 40C, D). PK2 infusion strongly suppresses REM sleep for the remaining 10 hours of the light period following injection, to 43% of control levels (p=0.018); a non-significant rebound of REM sleep (p=0.161) occurs in the following dark period (FIG. 40A, B). Total daily REM sleep time is unchanged (p=0.058). The effect of PK2 injection on NREM sleep time is smaller in comparison with the effect on REM sleep time. Over the 22 hours following injection, there is no significant change in total sleep time during the light and dark periods (p=0.090, 0.286, respectively).

Discussion: PK2 and aCSF were acutely infused into rats at two circadian time points, ZT2 or 2 hours after lights on, and ZT 14 or 2 hours after lights off, and NREM sleep, REM sleep, waking and active waking times were measured for 22 h following infusion. The most prominent effect of acute infusions of PK2 was on REM sleep amount. PK2 administration potently suppressed REM sleep for 10 hours, with the most marked suppression within the first 9 hours after ICV injection. This effect was present with injections at both ZT2 and ZT14. Following PK2 injections at ZT2, in the light period when sleep time and endogenous PK2 levels are maximal, REM sleep time, while substantial, was reduced to 50% of the control amount for the remainder of the light period following injection; infusion of PK2 at ZT14, in the dark period when sleep time and endogenous PK2 levels are minimal, nearly abolished the appearance of REM sleep for 10 hours following injection. The effects of acute PK2 administration on NREM sleep, however, are less pronounced. Following injection at ZT2, NREM sleep time was non-significantly reduced in the remainder of the light period, with no change in the following dark period. Following injection at ZT14, NREM sleep time was initially reduced, after which a small increase was observed in the remainder of the dark period, with no change in the following light period. Total NREM sleep time remained unchanged. It is possible that with a greater number of observations, an effect of NREM suppression following PK2 administration would emerge. Overall, acute PK2 administration at both ZT2 and ZT14 acted to flatten the circadian distribution of total sleep time.

REM sleep can be viewed as a period of central motor activation, and therefore, the suppression of REM sleep by PK2 and the suppression of motor activity by PK2 that we observed previously (Cheng et al., Nature JID-0410462 417: 405-410 (2002)) can be seen as related phenomena. It has been shown that the SCN acts to increase REM propensity during the light period, when endogenous PK2 expression is maximal; animals with intact SCNs, after being deprived of REM sleep, exhibit twice the REM tendency of SCN lesioned animals during the light period, but show no difference in REM propensity in the dark period (Wurts et al., J Neurosci JID-8102140 20:4300-4310 (2000)). Thus, endogenous levels of PK2 are not sufficient to suppress REM sleep, whereas we have shown that ICV infusions of 5 pg of PK2 potently suppress REM sleep. It is interesting to note that several drugs affecting the release or metabolism of monoamines produce REM sleep suppression of similar magnitude and have potent anti-depressant effects (Jones, B., In Principles and practice of sleep medicine. M. H. Kryger, T. Roth, and W. C. Dement, eds. (Philadelphia: W.B. Saunders Company), 145-162 (1994); Siegel, J. M. In Principles and Practice of Sleep Medicine. M. H. Kryger, T. Roth, and W. C. Dement, eds. (Philadelphia: W.B. Saunders Company), 125-144 (1994)).

### EXAMPLE XV

Generation and Characterization of PK2 Knockout (PK2−/−) Mice

The use of gene targeting by homologous recombination to generate knockout mouse models has proven to be extremely useful tool for drug target validation. This example describes the generation and characterization of PK2−/− mice. This study confirms that PK2 is essential for maintaining normal circadian locomotor rhythm. The wheel-running activity counts and the rhythmicity amplitude of PK2−/− mice are reduced by half compared to the values obtained for littermate controls under 12 hour light/dark cycle as well as under constant darkness (DD). In addition, preliminary feeding experiments also suggest that there is a significant reduction in daily food intake in PK2−/− mice compared with WT mice. As food intake is strongly controlled by the circadian clock coupled with the fact that acute PK2 infusion inhibits food take, PK2 may also have a key role in the circadian regulation of food intake.

Generation of PK2 knockout mice: A targeting vector (two exons of mouse PK2 gene were deleted) was constructed and transfected into ES cells by electroporation. Selection of the ES cells was carried out with G418 (200 μg/ml). The clones surviving the selection were expanded for freezing and DNA preparation. The ES clones that underwent homologous recombination were identified by Southern blot analysis of the ES cell DNA. To produce PK2−/− mice, chimeric mice were generated by microinjection of targeted ES cell clones. Chimeric male mice with agouti coat color of 25% or higher were tested for the germline transmission of agouti coat color. Germline transmission was confirmed by Southern blot hybridization with DNA prepared from tail biopsy samples. Heterozygous PK2+/−mice were then intercrossed to obtain homozygous PK2−/− mice. The genotype of PK2−/− mice were performed by PCR and confirmed by Southern blot analysis of DNA prepared from tail biopsy sample.

The low ratio of PK2−/− (17/160, 11%) indicates about half of the PK2 knock-out mice may succumb before weaning. This effect may be due to impaired olfactory bulb (OB) development; the OB from knockout mice is half of the size of WT mice and the OB is known to be important for mother/pup interaction.

Locomotor rhythmicity of PK2−/− mice in running-wheel cages: To assess the circadian locomotor rhythmicity, adult PK2−/− and their littermate wild type mice (age range of 3-6 months) were housed in individual running-wheel cages. The animals were placed in cycles of 12 hour Light/Dark Conditions (LD) for 4 weeks to study their entrained wheel-running behavior, and then in constant darkness (DD, dim light <1 lux) for an additional four to five weeks. Wheel-running activity was monitored continuously with a PC system (Minimitter Corp). The strength of the circadian rhythmicity was assessed with visual examination and then further analyzed by Fourier fast transformation (FFT) computer software. The wheel-running counts per period were also quantified.

Feeding behavior of PK2−/− mice: The food intake of PK2−/− and their littermate wild type mice (age 3-6 months) was monitored weekly. Running-wheels were present in the housing cages. The mice were under a 12 hr light/12 hr dark cycle and had free access to food (regular mouse chow) and water.

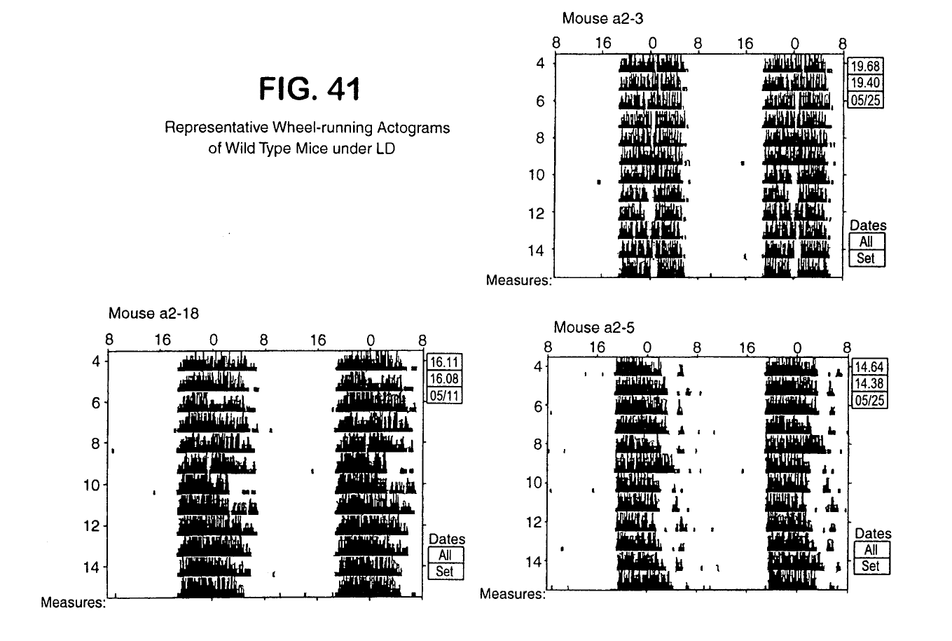

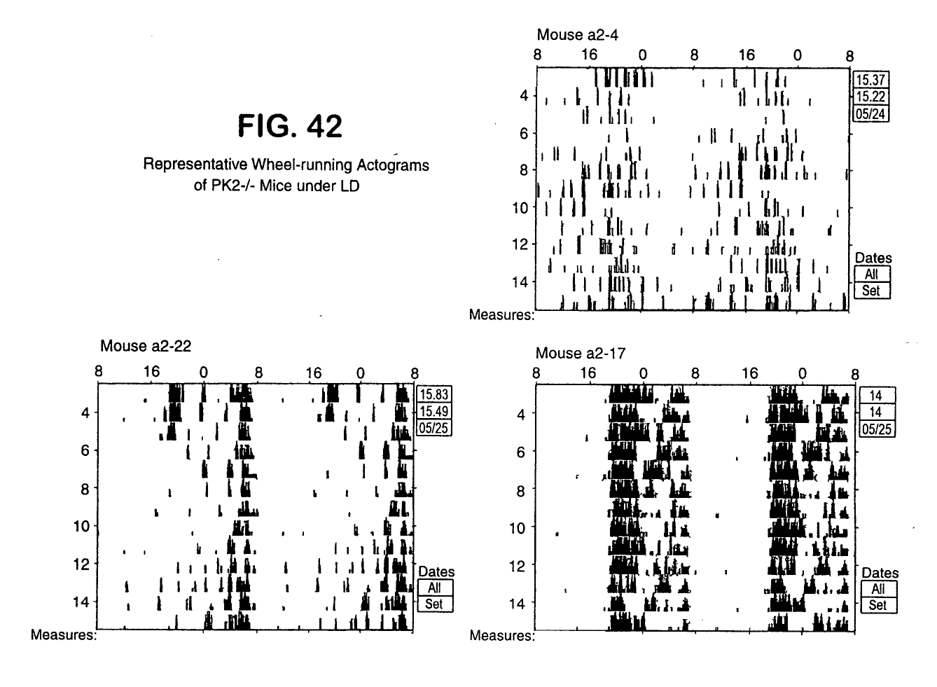

Reduced wheel-running activity and rhythm in PK2−/− mice under LD cycle: Representative wheel-running actograms of PK2−/− and their littermate controls are shown in FIG. 42 and FIG. 41, respectively.

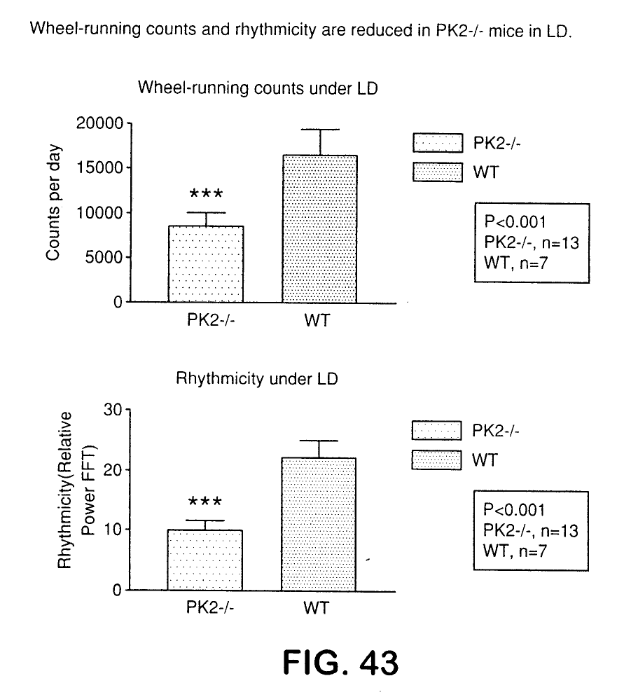

Visual inspection indicates that the rhythmicity is reduced in PK2−/− mice: Under LD, PK2−/− mice have about half of the wheel-running counts per 24 hour period, compared to their littermate control (FIG. 42). The amplitude of rhythmicity is also reduced (FIG. 43). Upon quantification by FFT (Fourier fast transformation) analysis, the rhythmicity amplitude of PK2−/− mice is found to be about half that of the wild type mice (relative power 9.9±1.6, n=13 for PK2 −/−, 22±2.9, n=7 for WT, p<0.001).

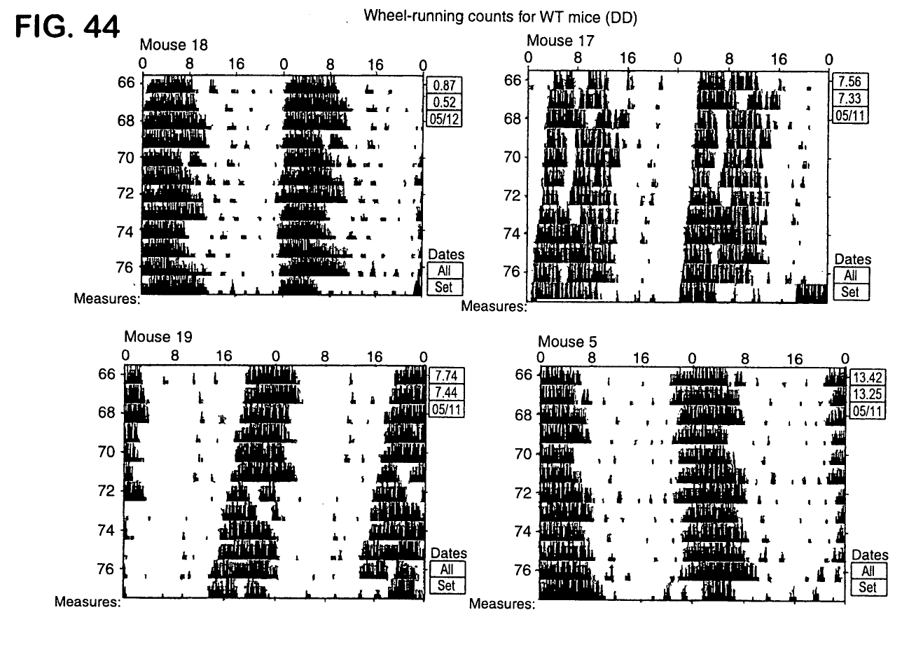

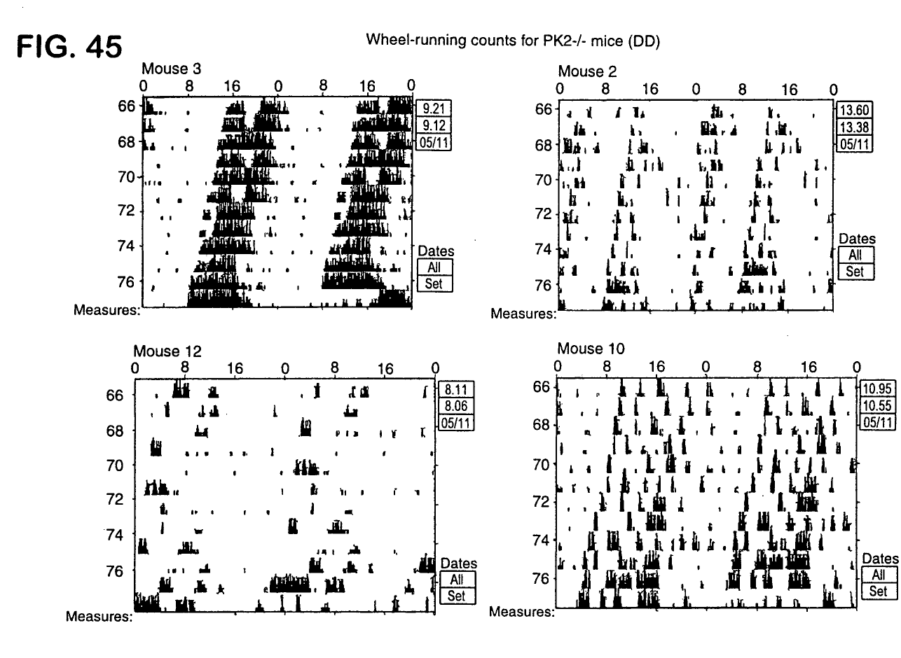

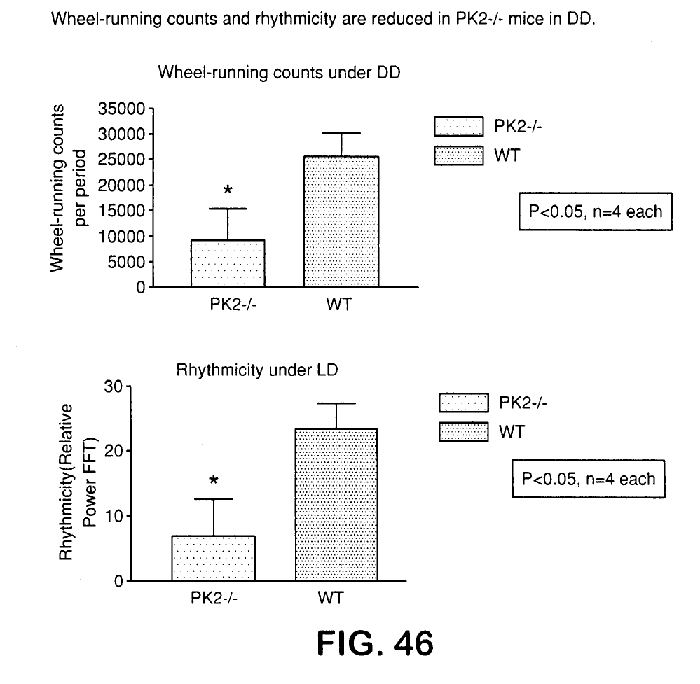

Reduced wheel-running activity and rhythm in PK2−/− mice under constant darkness (DD): The wheel-running activity of wild type (FIG. 44) and knockout mice (FIG. 45) were determined under constant darkness conditions. Under DD, the wheel-running counts of Pk2−/− mice are reduced (FIG. 46) compared to WT mice. The strength of rhythmicity is also significantly reduced (FIG. 46).

Compared to LD conditions (FIG. 43), there appears to be a further reduction in relative power of rhythmicity for PK2−/− under DD (FIG. 46), whereas the amplitude of rhythmicity is maintained.

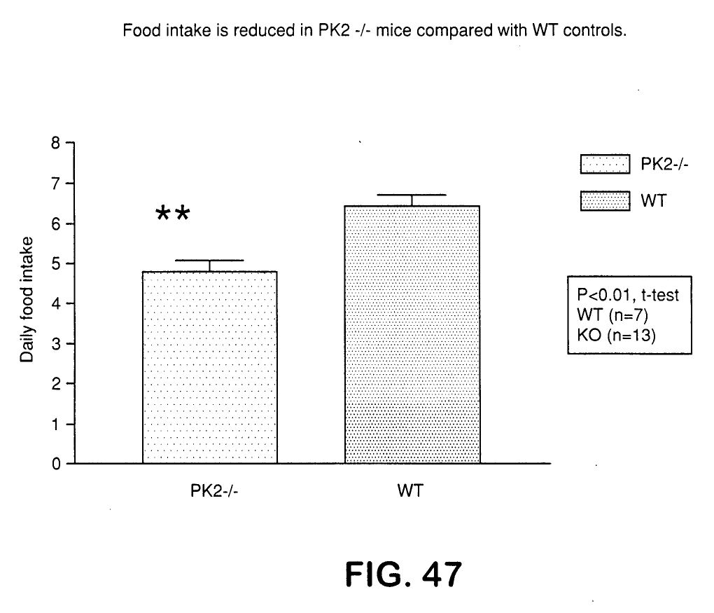

Reducedfood intake in PK2−/− mice under a 12 hour LD cycle. The food intake of PK2−/− mice and control mice was also monitored under LD conditions for two weeks. FIG. 47 shows that there was a significant reduction in food intake in PK2−/− mice compared with wild type mice. A reduction in food intake is also observed when PK2 is administered by icv injection into normal mice. The reason for these counter-intuitive finding is not understood at this time.

PK2−/− mice were generated and characterized in terms of their circadian wheel-running and food intake activities. Based on the results presented here, we conclude that PK2 is essential for maintaining normal circadian locomotor rhythm. This conclusion is based on the following observations:
The wheel-running activity counts and the rhythmicity amplitude of PK2−/− mice are reduced in half compared to the values obtained for littermate controls under both LD.
A similar reduction in wheel-running activity counts and the rhythmicity amplitude of PK2−/− mice vs. littermate controls is also observed under DD.

In addition, preliminary feeding experiments suggest that there is a significant reduction in daily food intake in PK2−/− mice compared with WT mice. As food intake is strongly controlled by the circadian clock coupled with the fact that acute PK2 infusion inhibits food take, PK2 may also have a key role in the circadian regulation of food intake.

### EXAMPLE XVI

Percent Body Fat in PK2 Knock-Out and Wild Type Mice

PK2−/− mice were obtained analogously to the method of Example XV above.

The protocolfor % bodyfat content measurement is as follows: Body Fat Measurement. Mouse carcass analysis was performed as follows. After removal of GI contents and other peripheral organs (heart, lung, spleen, kidneys), the dry weight of the carcass was determined. The carcasses were digested in 50 ml of alcoholic KOH (10% KOH in ethanol) in a Nalgene bottle at 70° C. for 2 hours. The bottles were then left at room temperature for 2-3 days to complete the saponification. Subsequently, MgCl₂ was added to 500-μl aliquots from the carcass digests to a final concentration of 0.5 M. The samples were placed on ice for 10 minutes, spun for 15 minutes in an Eppendorf microcentrifuge at maximal speed. The supernatants were assayed for fat content using the GPO-Trinder reagent (Sigma Chemical Co.). Triglyceride concentrations were estimated by measuring sample absorbance at 562 nm, in comparison with absorbance values of the known standards. After the standard curve is generated, the data are extrapolated from the standard curve in Prism. To determine total body fat content, the data were normalized by dividing over the carcass body weight to determine the % of total body fat content.

Following the methodology set forth above, the body fat content of PK2 knock-out mice was determined and compared with body fat content of wild type mice. The results of this experiment are shown in Table 48. These results show that the PK2 knock-out mice have more body fat than wild type mice (22% vs 12% of body weight for PK2 knock-out and wild type mice, respectively). The mean body weight of prokineticin 2 knockout mice was somewhat reduced compared with wild type mice (28.6 g versus 30.1 g for PK2 knock-out and wild type mice, respectively).

Throughout this application various publications have been referenced within parentheses. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific experiments detailed are only illustrative of the invention. It should be understood that various modifications can be made without departing from the spirit of the invention.

1. A method for modulating REM sleep in a mammal comprising, administering to the mammal an effective amount of prokineticin 2 (PK2) receptor binding agent to modulate REM sleep in the mammal.

2. The method of claim 1, wherein the mammal is a nocturnal mammal.

3. The method of claim 2, wherein the modulating of REM sleep is suppressing REM sleep.

4. A method for identifying an agent that modulates REM sleep in a subject, comprising:
(a) contacting a sample comprising a PK2 receptor with at least one test agent, under conditions suitable for the PK2 receptor to bind to the test agent; and
(b) comparing the binding of the PK2 receptor to the test agent, wherein binding of the PK2 receptor to the test agent promotes a predetermined signal as compared to a PK2 receptor modulating agent,
thereby identifying an agent that modulates REM sleep.

5. The method of claim 4, wherein the agent which binds to the PK2 receptor is administered to a subject to modulate REM sleep.

6. The method of claim 4, wherein the receptor is contacted with two or more agents.

7. The method of claim 4, wherein the receptor is a mouse PK2 receptor.

8. The method of claim 4, wherein the receptor is a primate PK2 receptor.

9. The method of claim 4, wherein the PK2 receptor modulating agent is human PK2.

10. The method of claim 2, wherein the animal is a mammal.

11. The method of claim 4, wherein the subject is selected from human, non-human primate, rat and mouse.

12. The method of claim 4, wherein the indicia of REM sleep is detected by EEG and EMG waves.
